(19) United States
(12) Patent Application Publication
(10) Pub. No.: US 2026/0274811 A1
(43) Pub. Date: Sep. 17, 2026

(54) SIGMA-1 RECEPTOR LIGANDS AND USE THEREOF

(71) Applicant: Xibin LIAO, Edison, NJ (US)

(72) Inventor: Xibin LIAO, Edison, NJ (US)

(21) Appl. No.: 19/165,004

(22) PCT Filed: Mar. 17, 2024
(86) PCT No.: PCT/US2024/020333

§ 371 Date: Sep. 12, 2025

Related U.S. Application Data
(60) Provisional application No. 63/452,828, filed on Mar. 17, 2023.

(51) Int. Cl. C07D 307/24; A61K 31/341; A61K 31/385; A61K 31/404; A61K 31/4525; A61K 31/496; C07D 307/14; C07D 405/06; C07D 405/12; C07D 409/12

(52) U.S. Cl. CPC C07D 307/24; A61K 31/341; A61K 31/385; A61K 31/404; A61K 31/4525; A61K 31/496; C07D 307/14; C07D 405/06; C07D 405/12; C07D 409/12

(57) ABSTRACT

A compound has the following formula (I), an isomer thereof, or a pharmaceutical acceptable solvate thereof. The compound of formula (I) is a potent and selective sigma-1 receptor ligand, and can treat Sigma-1 receptor related disorders or diseases.

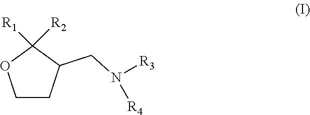

This application claims priority to U.S. Provisional Patent Application No. 63/452,828, filed on Mar. 17, 2023, which is incorporated by reference for all purposes as if fully set forth herein.

### FIELD OF THE INVENTION

The present invention relates to sigma-1 receptor ligands that provide high affinity binding to sigma-1 receptors.

### BACKGROUND OF THE INVENTION

The sigma-1 receptor is a chaperone protein at the endoplasmic reticulum that modulates calcium signaling through the inositol trisphosphate receptor receptor. In humans, the sigma-1 receptor is encoded by SIGMAR1 gene.

Sig-1R is implicated in multiple cellular processes, such as calcium modulation through inositol triphosphate receptor stabilization, endoplasmic stress regulation with the formation of a complex with BiP protein, and cell apoptosis and survival. Evidence supports a neuroprotective role of Sig-1R activation, by administering an agonist, on neurodegenerative disorders including Alzheimer's disease, Parkinson's disease, Huntington's disease, ALS, stroke, retinal degeneration, and depression. On the other hand, blocking Sig-1R by administration of antagonists is shown to be useful for the amelioration of psychosis, pain, drug abuse, and cancer.

The endogenous hallucinogen blarcarmesine (N,N-dimethyltrytamine, DMT) extracted from several plants is a Sig-1R regulator. DMT is rapidly metabolized in human body and it also has very limited brain-penetration. Given the therapeutic potential of DMT and its analogs for many disorders, there is a need for potent and selective Sig-1R ligands with improved bioavailability, extended or modified PK and PD to improve the therapeutic efficacy and safety window.

### SUMMARY OF THE INVENTION

The present invention provides a compound having the following formula (I), an isomer thereof, or a pharmaceutical acceptable salt thereof.

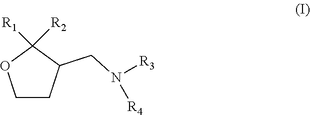

In formula (I), R₁ is

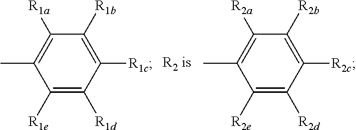

R₁ₐ, R_{1b}, R_{1c}, R_{1d}, R₁ₑ, R₂ₐ, R_{2b}, R_{2c}, R_{2d}, and R₂ₑ are each independently selected from the group consisting of —H, —F, —Cl, —Br, —I, —OH, —OCH₃, —CH₃, —CF₃, —CHF₂, —CH₂F, —CD₃, —CN, and

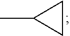

R₃ and R₄ are independently selected from the group consisting of —CH₃, —CD₃, —CF₃, —CHF₂, —CH₂F, —(CH₂)ₙ—O—R₅, and —CO—R₆, n is 1 or 2, provided that when R₁ₐ, R_{1b}, R_{1c}, R_{1d}, R₁ₑ, R₂ₐ, R_{2b}, R_{2c}, R_{2d}, and R₂ₑ are all —H, at least one of R₃ and R₄ is —CD₃, —CF₃, —CHF₂, —CH₂F, —(CH₂)ₙ—O—R₅, or —CO—R₆; or R₃ and R₄ form a 5-membered or 6-membered heterocyclyl group; and R₅ is

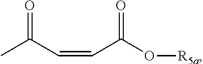

—CO—CH(R_{5b})—(CH₂)ₘ—R_{5c}, or —CH₂—R_{5d}; R₅ₐ is —H or a C1-6 alkyl group; R_{5b} is —H, —NH₂ or

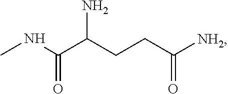

m is 0, 1, 2, or 3, R_{5c} is —COOH, —CONH₂, —NH₂, —NH—CO—CH₂—NH₂,

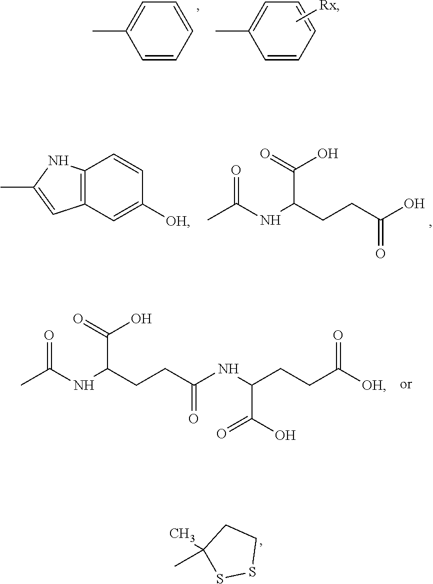

Rₓ is —OH, —NH₂, —F, —Cl, —Br, or —CN; R_{5d} is —CH₂CH₂NH₂, —CH₂CH₂CONH₂,

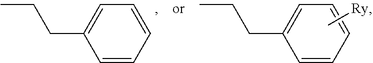

R_{y} is —OH, —NH₂, —F, —Cl, —Br, or —CN; and R₆ is —CH₂CH₂NH₂, —CH₂CH₂CONH₂,

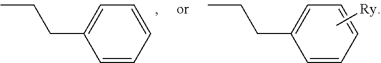

In another embodiment, in formula (1), R₁ is

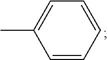

### R₂ is

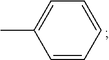

R₃ is selected from the group consisting of —CD₃, —CF₃, —CHF₂, and —CH₂F; and R₄ is selected from the group consisting of —CH₃, —CD₃, —CF₃, —CHF₂, and —CH₂F.

In another embodiment, the compound is the selected from the group consisting of

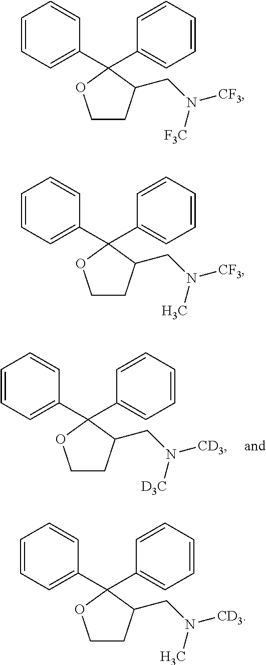

In another embodiment, in formula (1), R₁ is

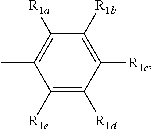

one of R₁ₐ, R_{1b}, R_{1c}, R_{1d}, and R_{1c} is selected from the group consisting of —F, —Cl, —Br, —I, —OH, —OCH₃, —CH₃, —CF₃, —CHF₂, —CH₂F, —CD₃, —CN, and

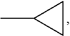

and remaining four of R₁ₐ, R_{1b}, R_{1c}, R_{1d}, and R_{1c} are selected from the group consisting of —H, —F, —Cl, —Br, —I, —OH, —OCH₃, —CH₃, —CF₃, —CHF₂, —CH₂F, —CD₃, —CN; and/or R₂ is

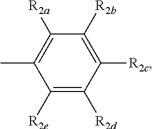

one of R₂ₐ, R_{2b}, R_{2c}, R_{2d}, and R₂ₑ is selected from the group consisting of —F, —Cl, —Br, —I, —OH, —OCH₃—CH₃, —CF₃, —CHF₂, —CH₂F, —CD₃, —CN, and

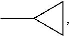

and remaining four of R₂ₐ, R_{2b}, R_{2c}, R_{2d}, and R₂ₑ are selected from the group consisting of —H, —F, —Cl, —Br, —I, —OH, —OCH₃, —CH₃, —CF₃, —CHF₂, —CH₂F, —CD₃, —CN.

In another embodiment, in formula (1), the remaining four of R₁ₐ, R_{1b}, R_{1c}, R_{1d}, and R₁ₑ are —H; and/or the remaining four of R₂ₐ, R_{2b}, R_{2c}, R_{2d}, and R₂ₑ are —H.

In another embodiment, in formula (1), R₃ and R₄ form a group selected from the group consisting of

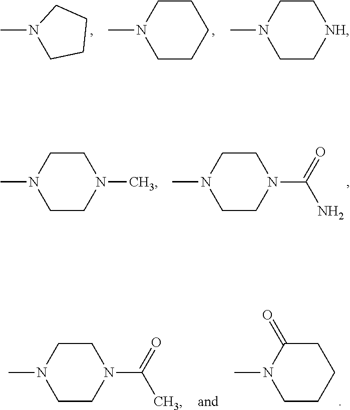

In another embodiment, in formula (1), R₃ is —CH₃, —CD₃, —CF₃, —CHF₂, or —CH₂F; R₄ is —CH₂—O—R₅; R₅ is

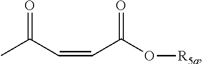

and R₅ₐ is —H, —CH₃, —CH₂CH₃, or —C(CH₃)₃.

In another embodiment, in formula (1), R₃ is —CH₃, —CD₃, —CF₃, —CHF₂, or —CH₂F; R₄ is —(CH₂)ₙ—O—R₅; R₅ is —CO—CH(R_{5b})—(CH₂)ₘ—R_{5c}; n is 1 or 2; m is 1 or 2; R_{5b} is —NH₂ or

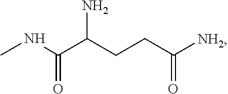

and R_{5c} is —COOH, —CONH₂,

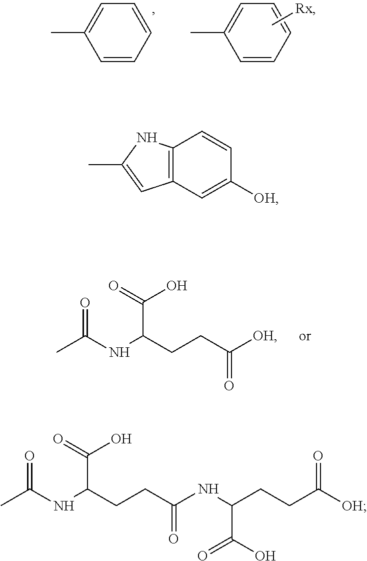

Rₓ is —OH, —NH₂, —F, —Cl, —Br, or —CN.

In another embodiment, in formula (1), R₃ is —CH₃, —CD₃, —CF₃, —CHF₂, or —CH₂F; R₄ is —CH₂—O—R₅; R₅ is —CO—CH₂—(CH₂)ₘ—R_{5c}; m is 0, 1, 2, or 3; R_{5c} is —NH₂, —NH—CO—CH₂—NH₂,

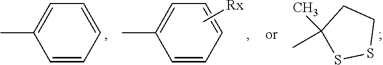

Rₓ is —OH, —NH₂, —F, —Cl, —Br, or —CN.

In another embodiment, in formula (1), R₃ is —CH₃, —CD₃, —CF₃, —CHF₂, or —CH₂F; R₄ is —CH₂—O—R₅; R₅ is —CH₂—R_{5d}; R_{5d} is —CH₂CH₂NH₂, —CH₂CH₂CONH₂,

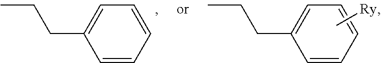

R_{y} is —OH, —NH₂, —F, —Cl, —Br, or —CN.

In another embodiment, in formula (1), R₃ is —CH₃, —CD₃, —CF₃, —CHF₂, or —CH₂F; R₄ is and —CO—R₆; R₆ is —CH₂CH₂NH₂,

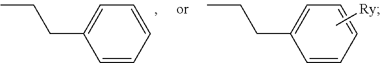

R_{y} is —OH, —NH₂, —F, —Cl, —Br, or —CN.

In another embodiment, the compound of formula (1) is selected from the group consisting of

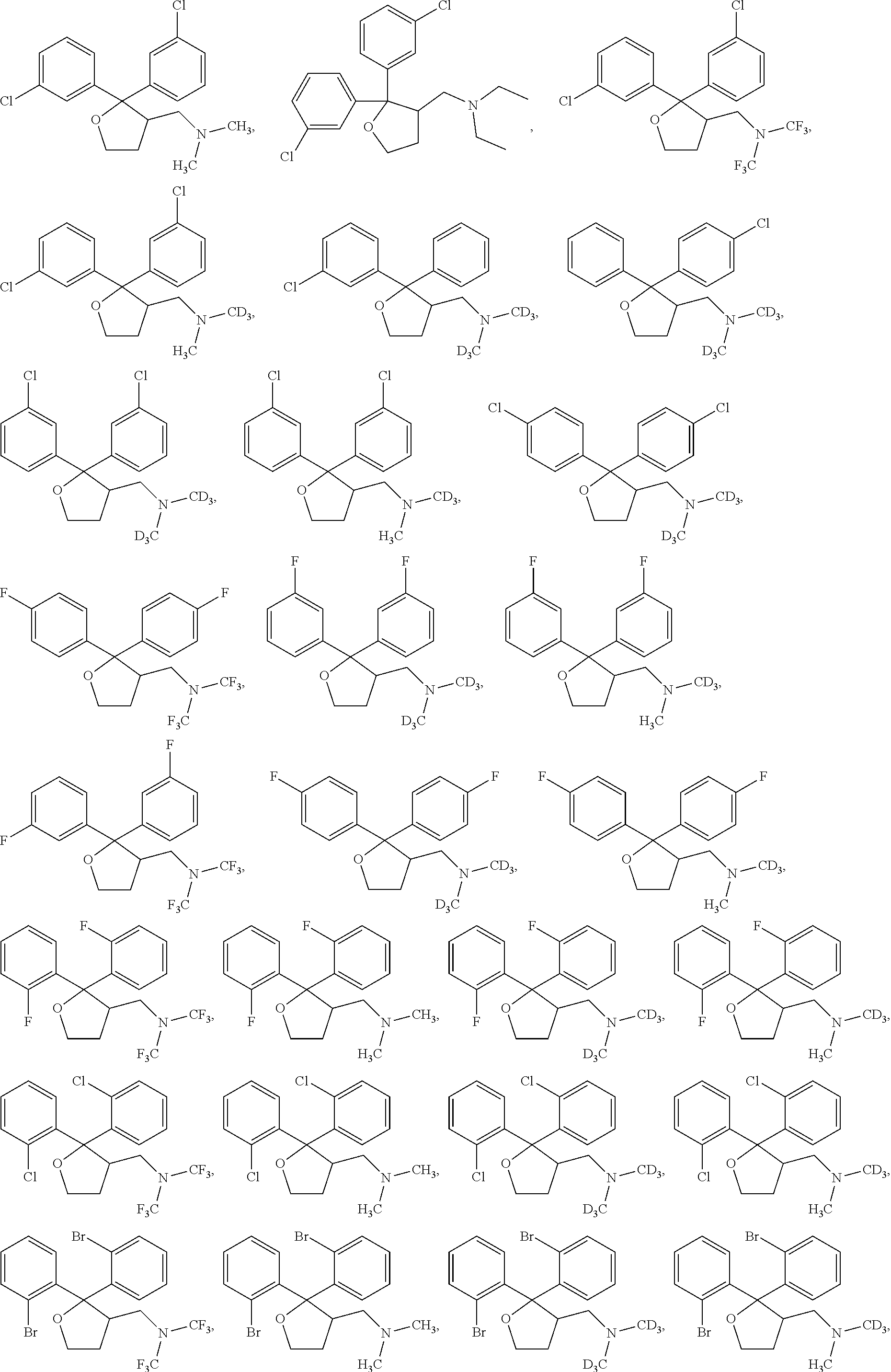

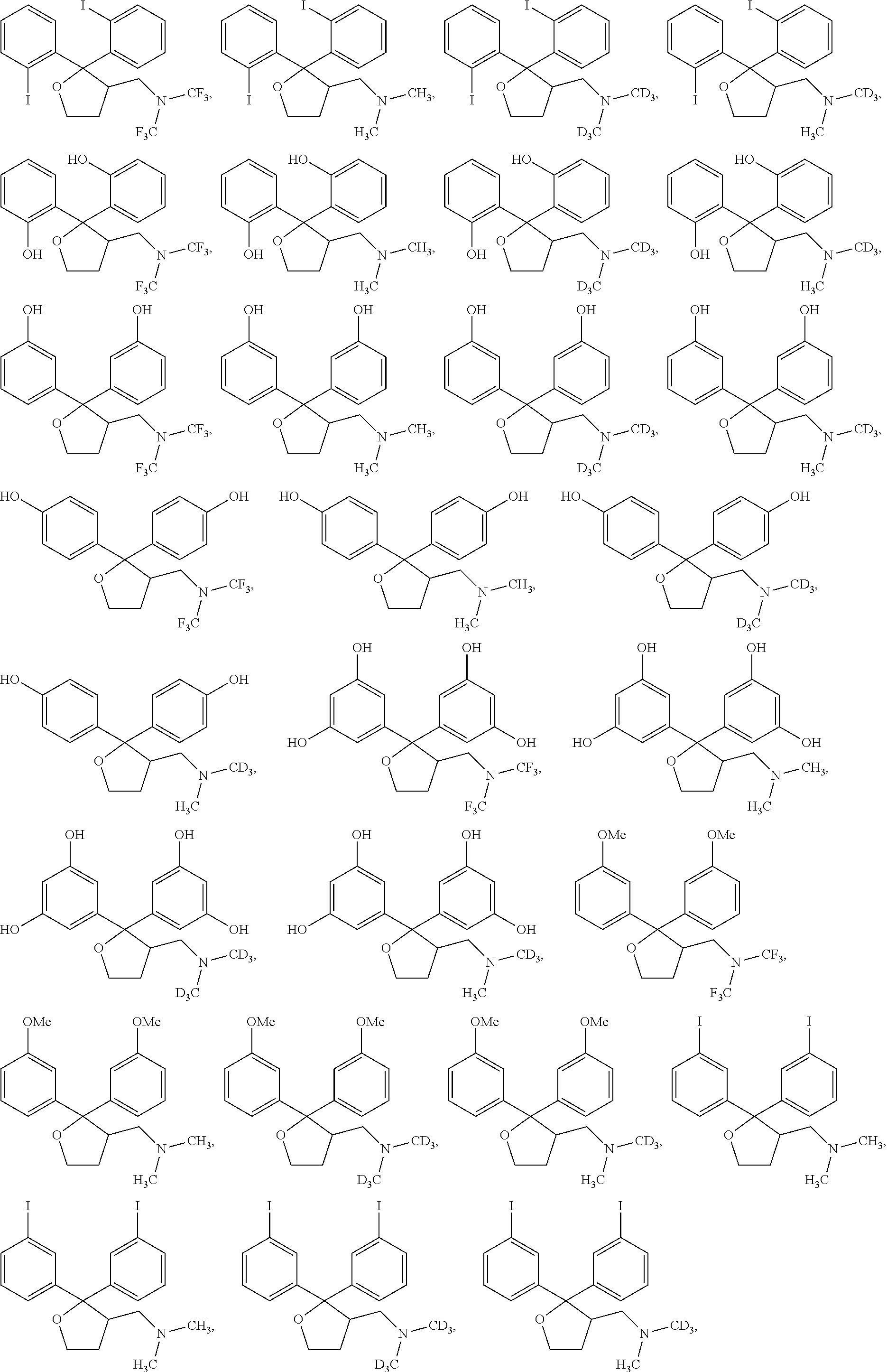

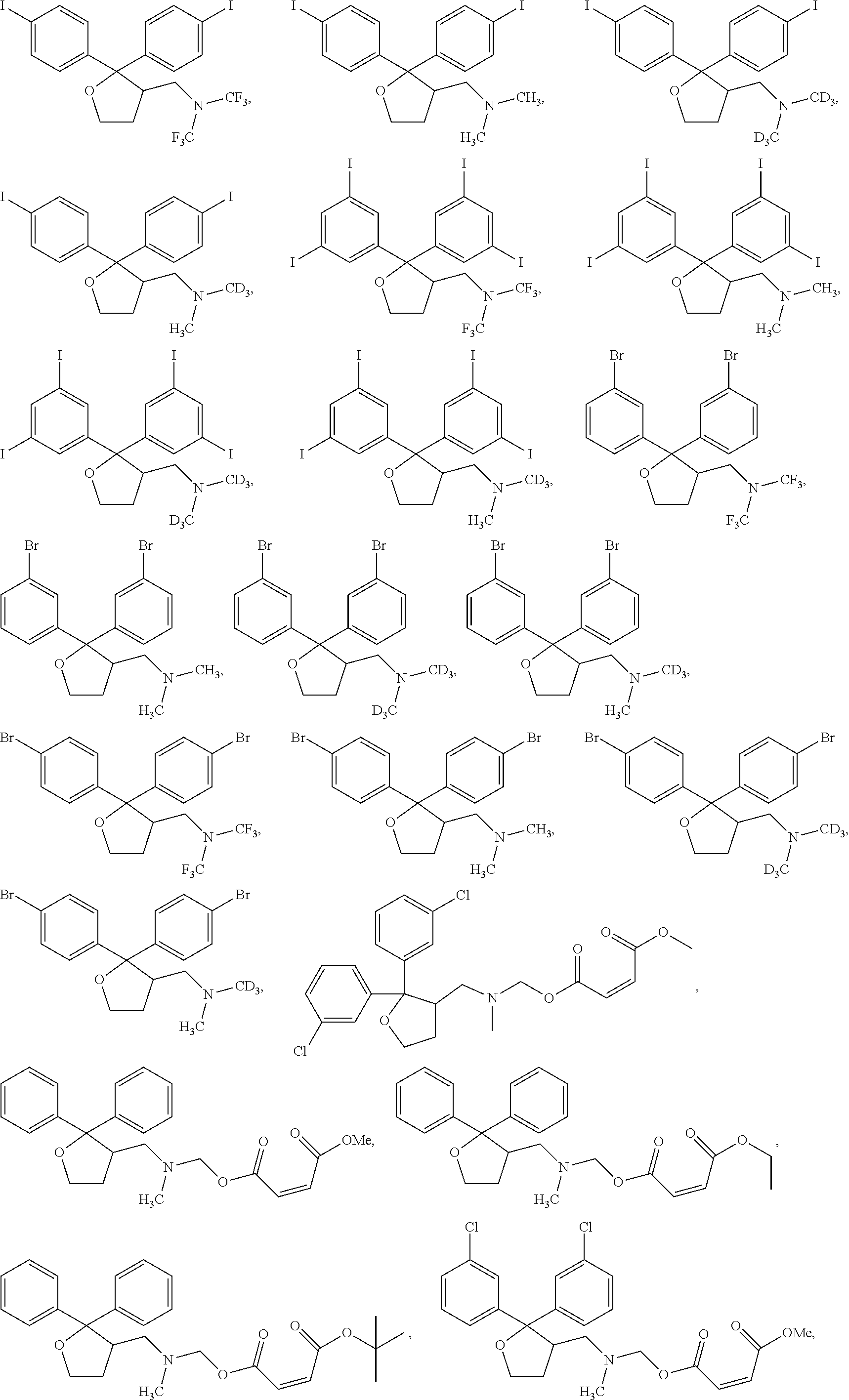

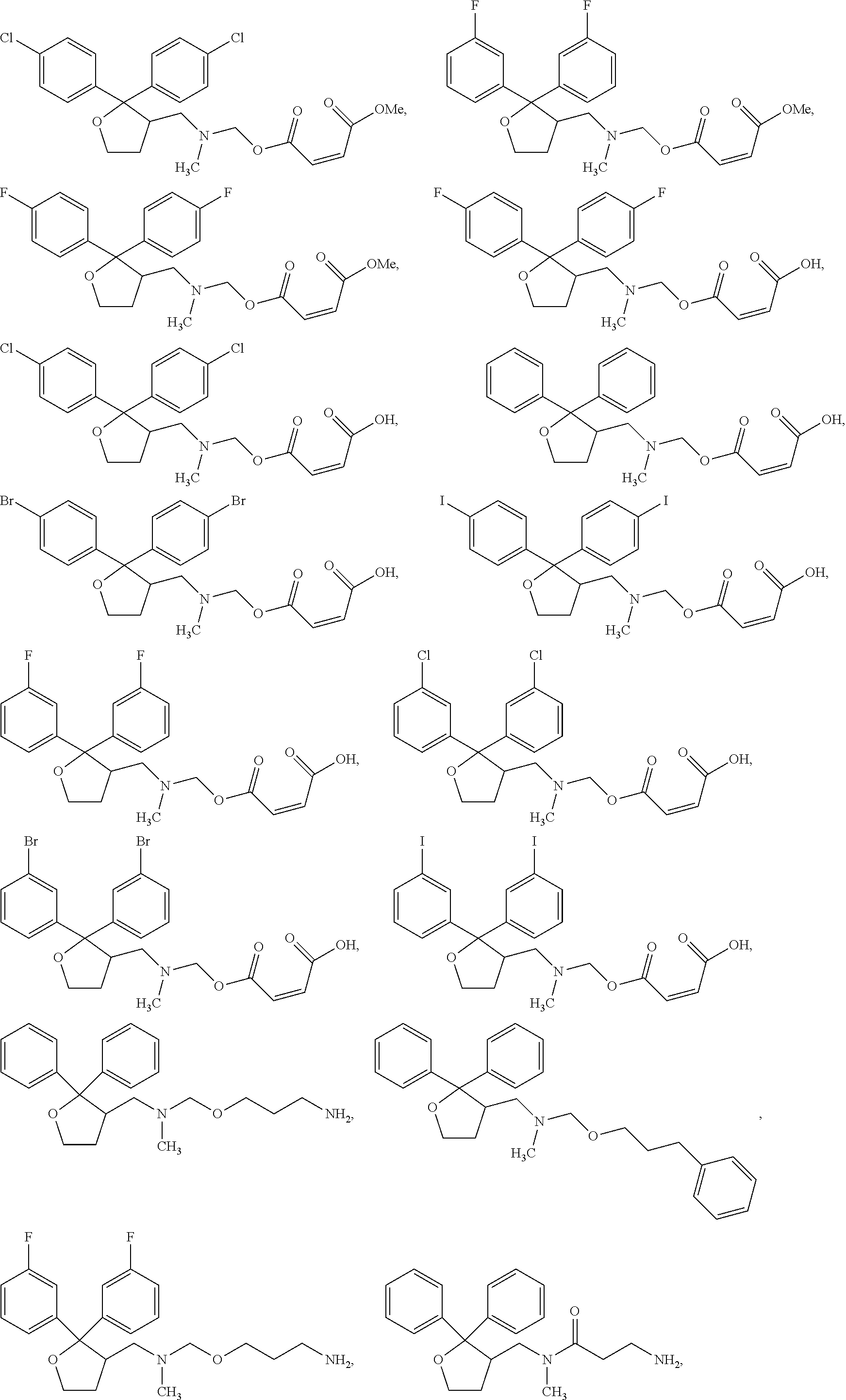

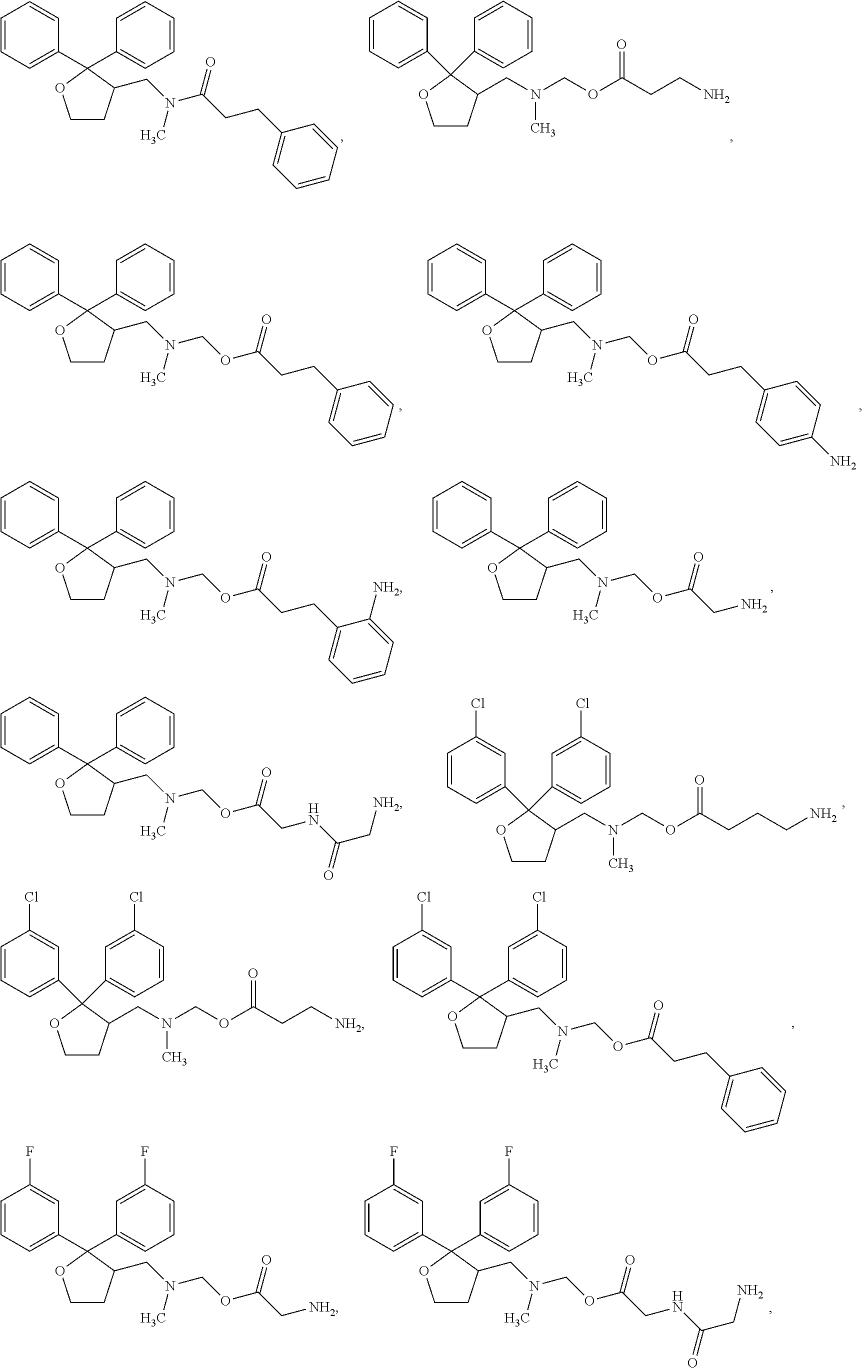

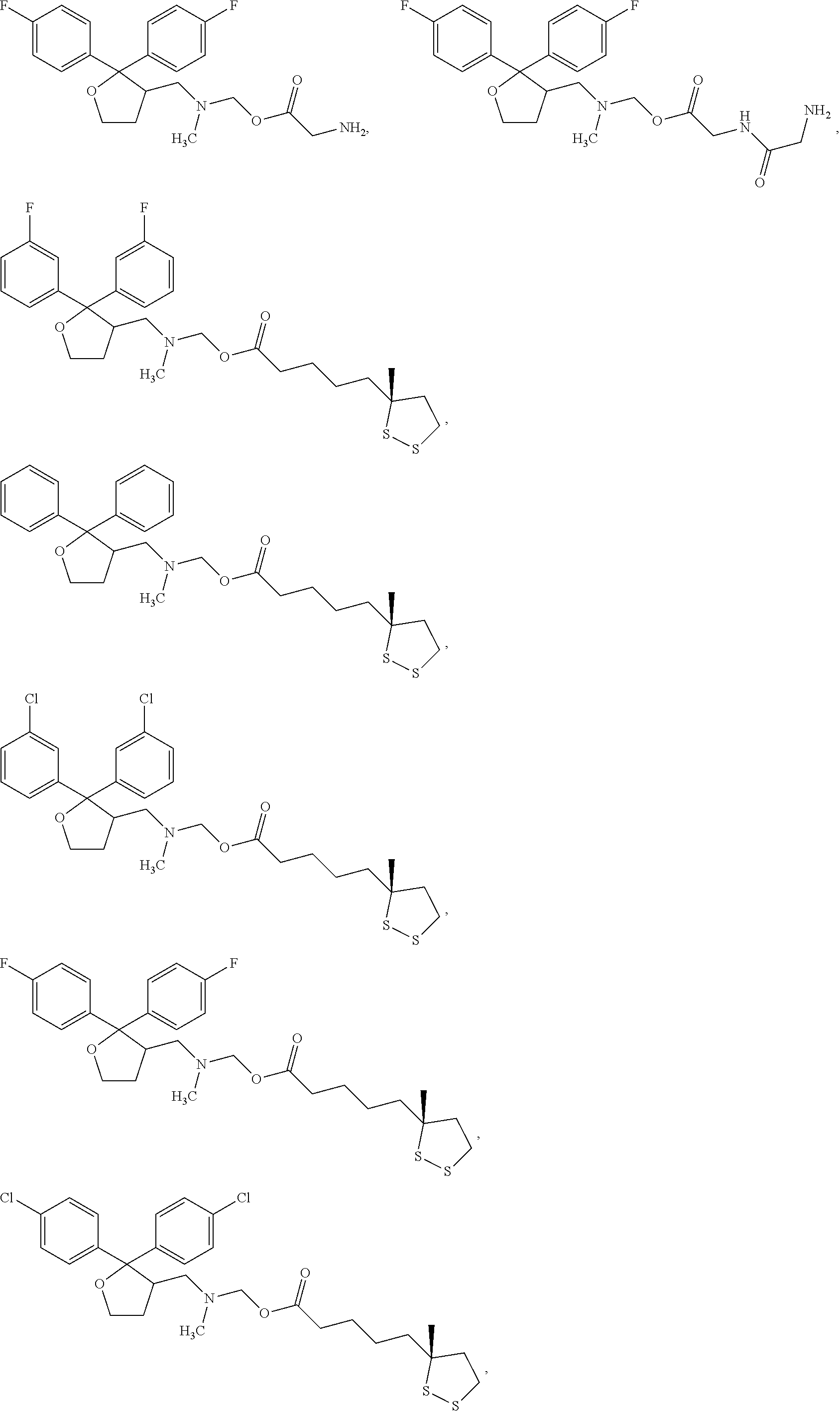

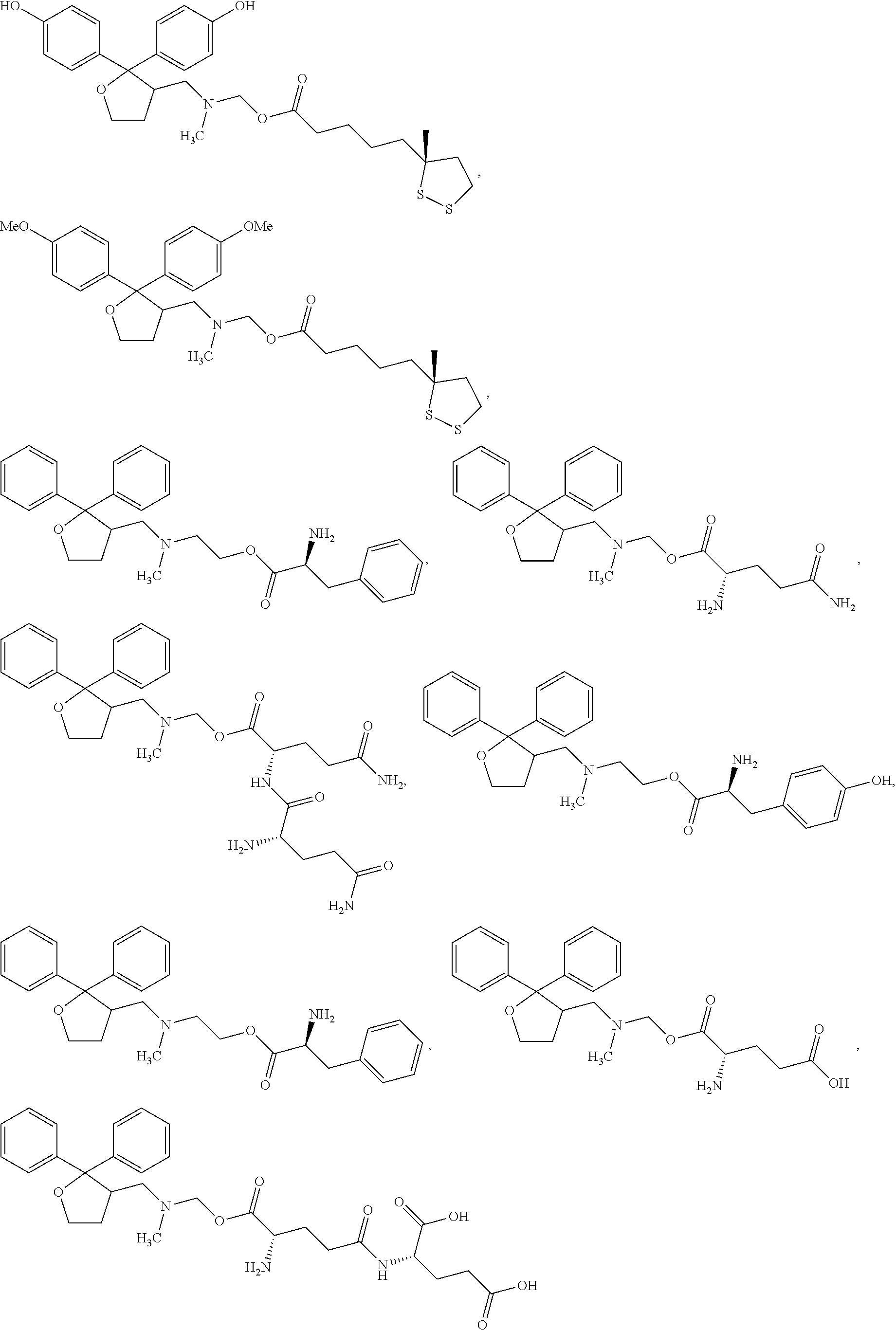

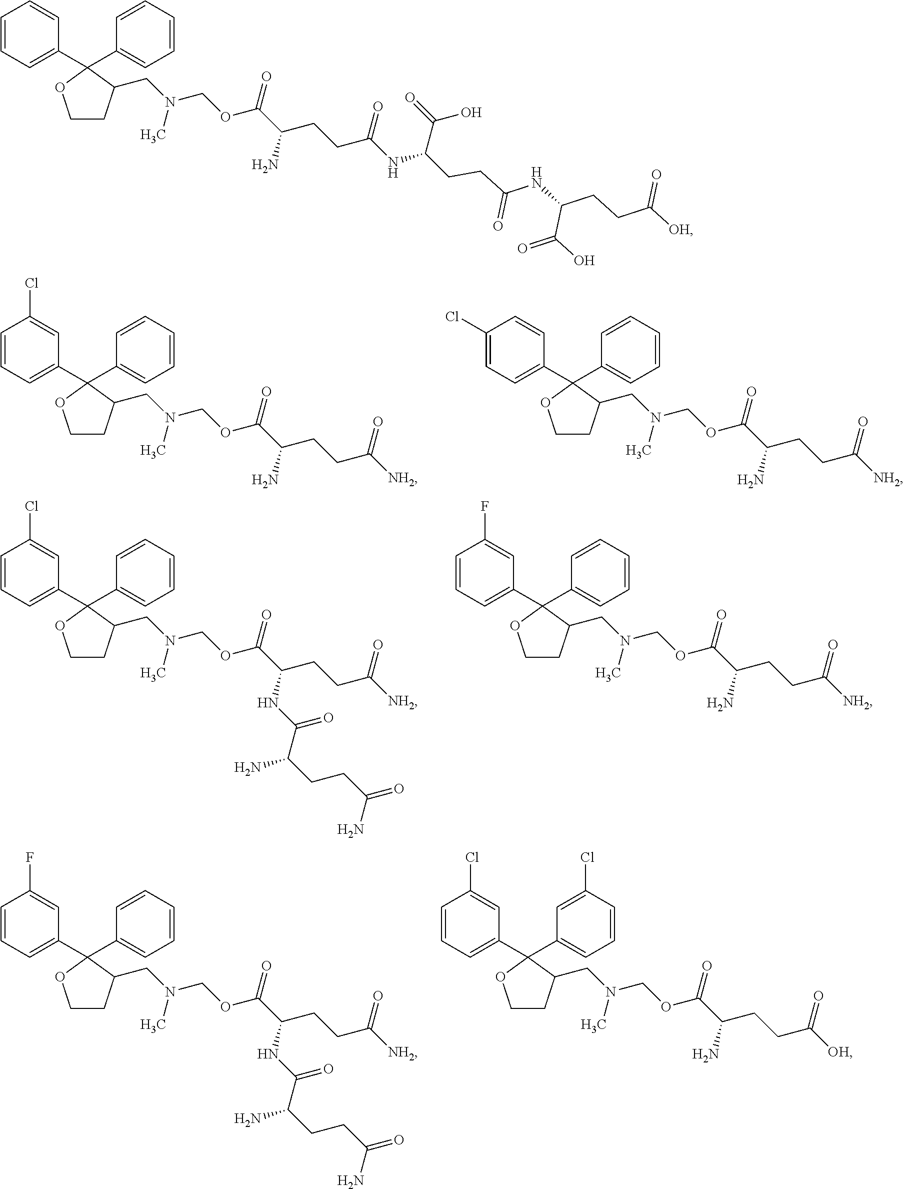

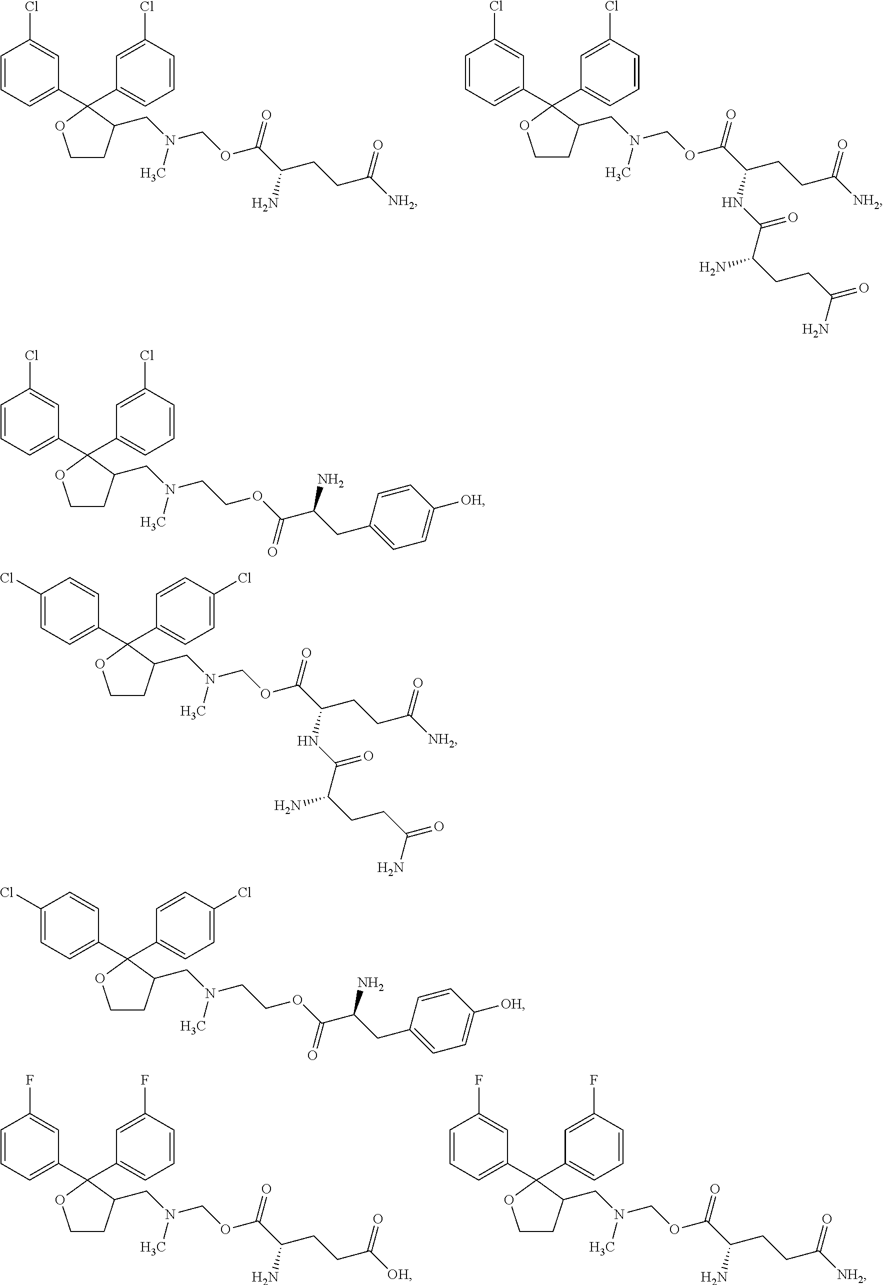

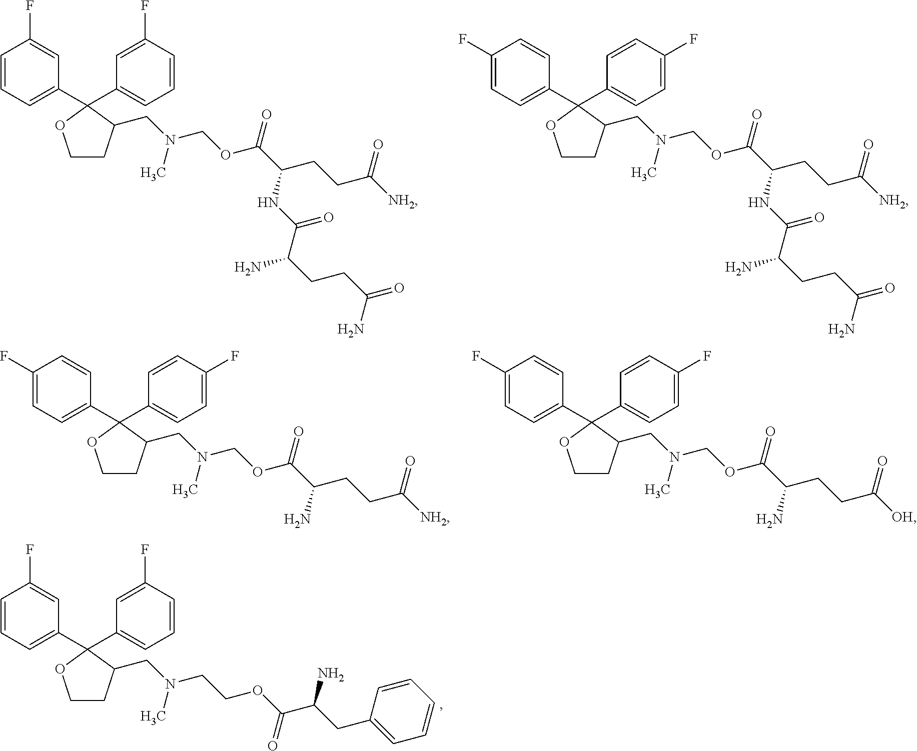 [SMILES: CN(COC(=O)[C@H](CCC(N)=O)NC(=O)[C@@H](N)CCC(N)=O)CC1CCOC1(c1cccc(F)c1)c1cccc(F)c1 ; CN(COC(=O)[C@H](CCC(N)=O)NC(=O)[C@@H](N)CCC(N)=O)CC1CCOC1(c1ccc(F)cc1)c1ccc(F)cc1 ; CN(COC(=O)[C@@H](N)CCC(N)=O)CC1CCOC1(c1ccc(F)cc1)c1ccc(F)cc1 ; CN(COC(=O)[C@@H](N)CCC(=O)O)CC1CCOC1(c1ccc(F)cc1)c1ccc(F)cc1 ; CN(CCOC(=O)[C@@H](N)Cc1ccccc1)CC1CCOC1(c1cccc(F)c1)c1cccc(F)c1]

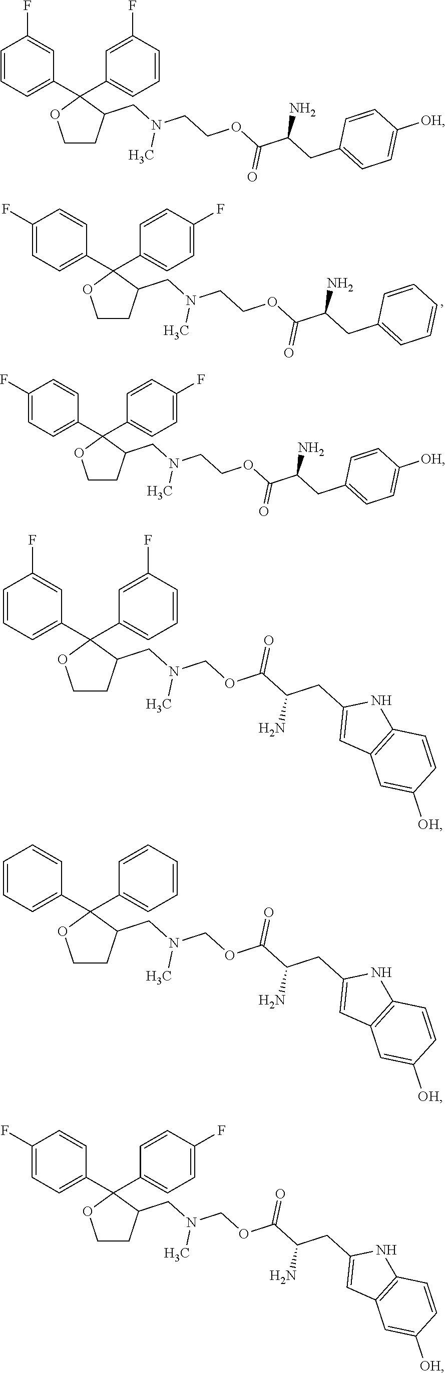 [SMILES: CN(CCOC(=O)[C@@H](N)Cc1ccc(O)cc1)CC1CCOC1(c1cccc(F)c1)c1cccc(F)c1 ; CN(CCOC(=O)[C@@H](N)Cc1ccccc1)CC1CCOC1(c1ccc(F)cc1)c1ccc(F)cc1 ; CN(CCOC(=O)[C@@H](N)Cc1ccc(O)cc1)CC1CCOC1(c1ccc(F)cc1)c1ccc(F)cc1 ; CN(COC(=O)[C@@H](N)Cc1cc2cc(O)ccc2[nH]1)CC1CCOC1(c1cccc(F)c1)c1cccc(F)c1 ; CN(COC(=O)[C@@H](N)Cc1cc2cc(O)ccc2[nH]1)CC1CCOC1(c1ccccc1)c1ccccc1 ; CN(COC(=O)[C@@H](N)Cc1cc2cc(O)ccc2[nH]1)CC1CCOC1(c1ccc(F)cc1)c1ccc(F)cc1]

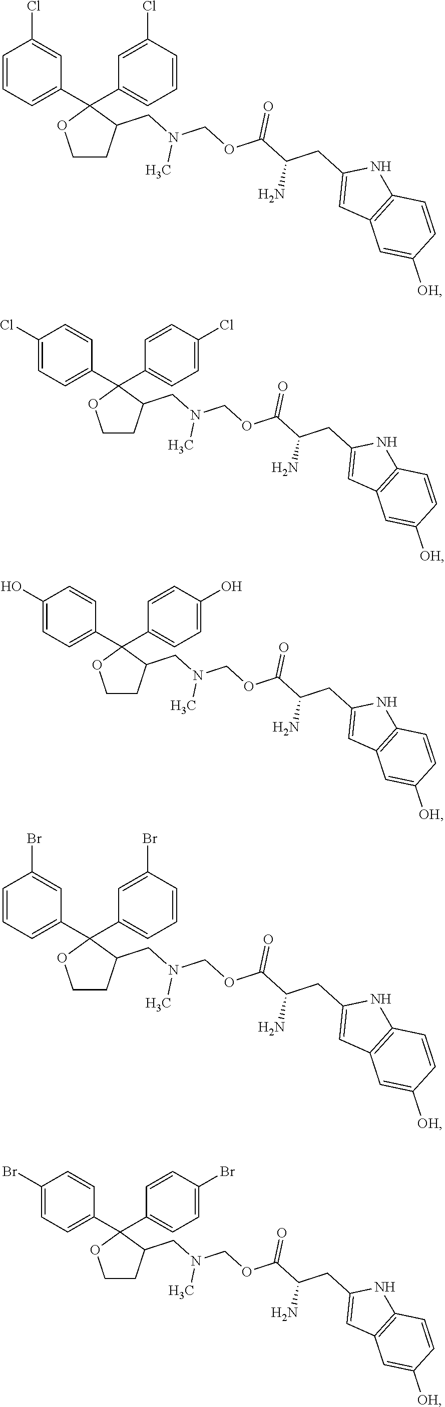 [SMILES: CN(COC(=O)[C@@H](N)Cc1cc2cc(O)ccc2[nH]1)CC1CCOC1(c1cccc(Cl)c1)c1cccc(Cl)c1 ; CN(COC(=O)[C@@H](N)Cc1cc2cc(O)ccc2[nH]1)CC1CCOC1(c1ccc(Cl)cc1)c1ccc(Cl)cc1 ; CN(COC(=O)[C@@H](N)Cc1cc2cc(O)ccc2[nH]1)CC1CCOC1(c1ccc(O)cc1)c1ccc(O)cc1 ; CN(COC(=O)[C@@H](N)Cc1cc2cc(O)ccc2[nH]1)CC1CCOC1(c1cccc(Br)c1)c1cccc(Br)c1 ; CN(COC(=O)[C@@H](N)Cc1cc2cc(O)ccc2[nH]1)CC1CCOC1(c1ccc(Br)cc1)c1ccc(Br)cc1]

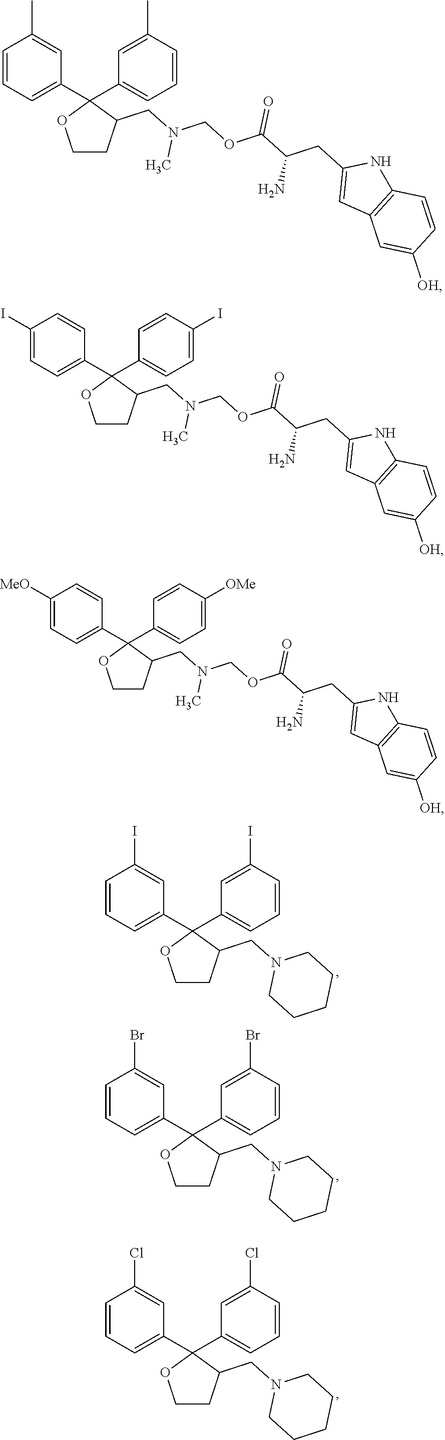

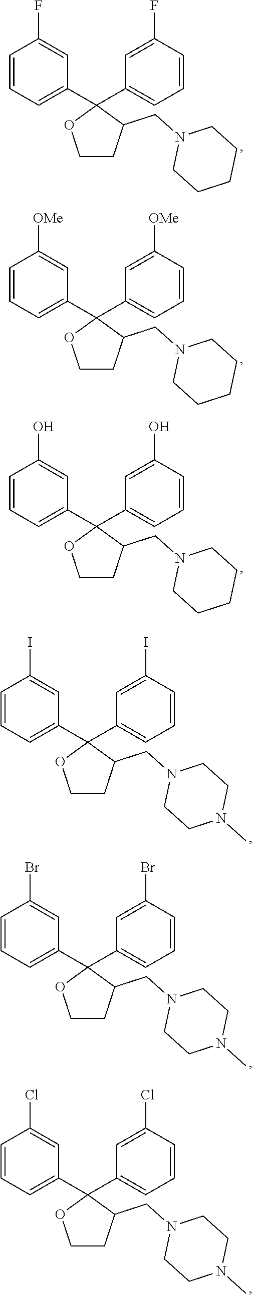 [SMILES: Fc1cccc(C2(c3cccc(F)c3)OCCC2CN2CCCCC2)c1 ; COc1cccc(C2(c3cccc(OC)c3)OCCC2CN2CCCCC2)c1 ; Oc1cccc(C2(c3cccc(O)c3)OCCC2CN2CCCCC2)c1 ; CN1CCN(CC2CCOC2(c2cccc(I)c2)c2cccc(I)c2)CC1 ; CN1CCN(CC2CCOC2(c2cccc(Br)c2)c2cccc(Br)c2)CC1 ; CN1CCN(CC2CCOC2(c2cccc(Cl)c2)c2cccc(Cl)c2)CC1]

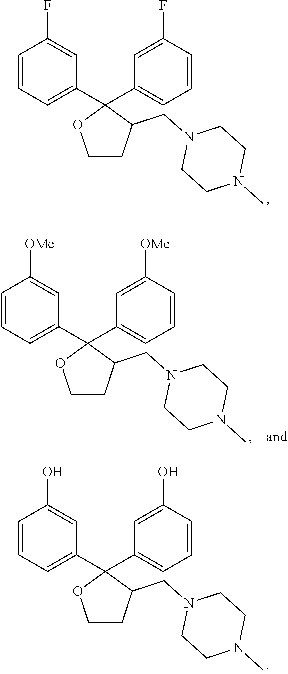 [SMILES: CN1CCN(CC2CCOC2(c2cccc(F)c2)c2cccc(F)c2)CC1 ; COc1cccc(C2(c3cccc(OC)c3)OCCC2CN2CCN(C)CC2)c1 ; CN1CCN(CC2CCOC2(c2cccc(O)c2)c2cccc(O)c2)CC1]

In another embodiment, in formula (1), the present application provides a pharmaceutical composition for treating Sigma-1 receptor related disorders or diseases. The the pharmaceutical composition includes the compound of formula (1).

### DETAILED DESCRIPTION OF THE INVENTION

The present invention provides a compound having the following formula (I), an isomer thereof, a tautomer thereof, a pharmaceutical acceptable salt thereof.

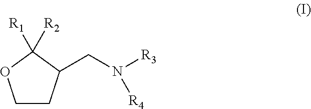

Compounds of formulas (I) may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formulas (I) and (II) may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and, unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers.

As used herein the following definitions are applicable.

“Alkyl,” as well as other groups having the prefix “alk,” such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures.

“Heterocyclyl” or “heterocyclo ring” refers to fully saturated or partially unsaturated non-aromatic cyclic groups which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and/or sulfur heteroatoms may optionally be oxidized and the nitrogen heteroatoms may optionally be quaternized. A heterocyclo ring may have a carbon ring atom replaced with a carbonyl group (C═O), as illustrated above for cycloalkyl groups.

The compounds of the present application may be prepared from known or readily prepared starting materials, following methods known to one skilled in the art of organic synthesis. Methods useful for making the compounds of Formula are set forth in the examples below and generalized in Schemes A, B, C below. Alternative synthetic pathways and analogous structures will be apparent to those skilled in the art of organic synthesis.

Scheme A shows a method useful for making final compounds of formula A5, which correspond to one of the compounds of Formula (I). In Scheme A, R₁ and R₂ are defined above in this application.

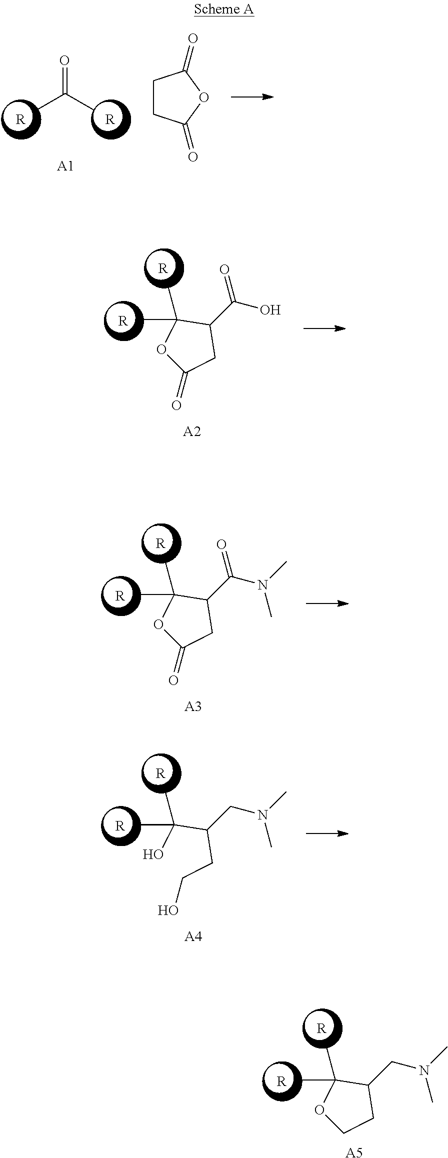

Compounds A1 are reacted with dione to provide compounds A2, and an amide formation provides compounds A3. Global reduction provides diol A4, and finally a cyclization provides compounds type A5.

Scheme B shows a method useful for making N-analogs compounds B4 and B6, which correspond to some compounds of Formula (II). In Scheme B, R₁, R₂, R₃ and R₄ are defined above in this application.

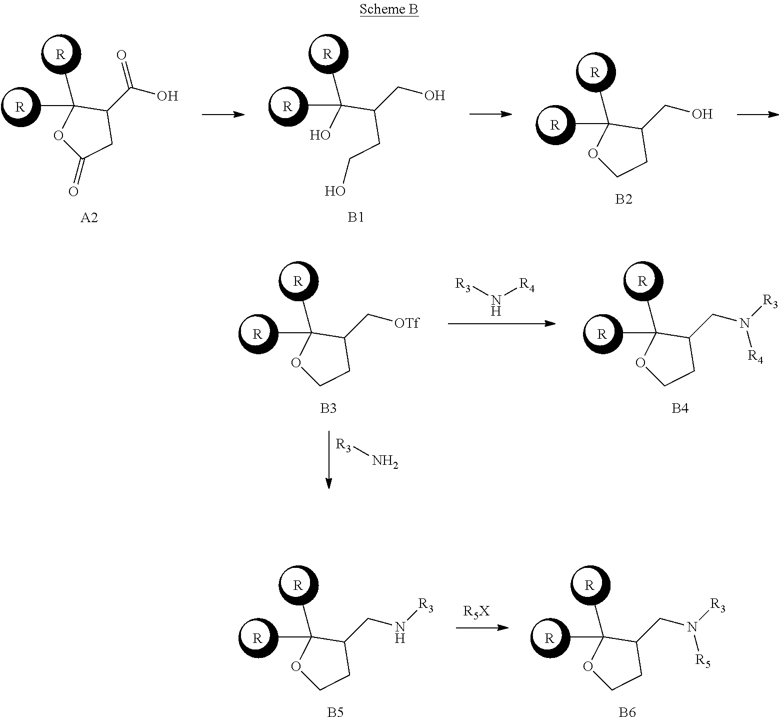

Compounds A2 are reduced to provide triol B1, then cyclization to provide compounds B2, further installing a leaving group to provide key intermediate B3, following react with primary or secondary amines to provide B4 and B5. B5 can react with R₅X to provide B6.

Scheme C shows a method useful for making ether or amide analogs compounds of formula C2, which correspond to some compounds of Formula (I). In Scheme C, R₅ is defined above in this application.

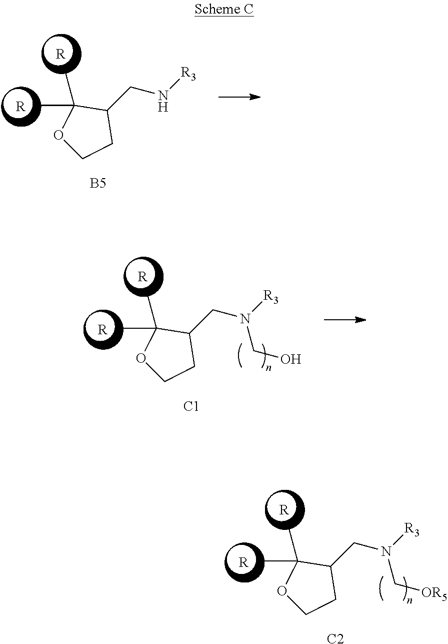

Compounds B3 are converted to C1, then converted to a variety of ester or ether as compounds C2.

### General Procedures:

Preparative thin layer chromatography (PTLC) was performed on 20×20 cm plates (500 micron thick silica gel). Silica gel chromatography was performed on a Biotage Horizon flash chromatography system.

¹H and ¹³C NMR spectra were recorded on a Bruker Ascend™ 400 spectrometer at 400 MHz at 298° K, and the chemical shifts are given in parts per million (ppm) referenced to the residual proton signal of the deuterated solvents: CHCl₃ at δ=7.26 ppm and CH3OH or CH3OD at δ=3.30 ppm.

LCMS spectra were taken on an Agilent Technologies 1260 Infinity or 6120 Quadrupole spectrometer. The mobile phase for the LC was acetonitrile (A) and water (B) with 0.01% formic acid, and the eluent gradient was from 5-95% A in 6.0 min, 60-95% A in 5.0 min, 80-100% A in 5.0 min and 85-100% A in 10 min using a SBC18 50 mm×4.6 mm×2.7 m capillary column.

Mass spectra (MS) were measured by electrospray ion-mass spectroscopy (ESI). All temperatures are degrees Celsius unless otherwise noted.

### Analytical HPLC Mass Spectrometry Conditions:

LC1: Column: SB-C18 50 mm×4.6 mm×2.7 μm
   Temperature: 50° C.
   Eluent: 5:95 v/v acetonitrile/water+0.01% formic acid in 6 min.
   Flow Rate: 1.5 mL/min, Injection 5 μL
   Detection: PDA, 200-600 nm
   MS: mass range 150-750 amu; positive ion electrospray ionization
LC2: Column: SB-C18 50 mm×4.6 mm×2.7 μm
   Temperature: 50° C.
   Eluent: 5:95 to 95:5 v/v acetonitrile/water+0.05% TFA over 3.00 min.
   Flow Rate: 1.5 mL/min, Injection 5 μL
   Detection: PDA, 200-600 nm
   MS: mass range 150-750 amu; positive ion electrospray ionization
LC3: Column: SB-C18 50 mm×4.6 mm×2.7 μm
   Temperature: 50° C.
   Eluent: 10:90 to 98:2 v/v acetonitrile/water+0.05% TFA over 3.75 min.
   Flow Rate: 1.0 mL/min, Injection 10 μL
   Detection: PDA, 200-600 nm
   MS: mass range 150-750 amu; positive ion electrospray ionization

The following abbreviations are used in the Examples, the Schemes, and the Tables:
AcOH=acetic acid
Aq=aqueous
Alk=alkyl
Ar=aryl
Boc=tert-butyloxycarbonyl
br=broad singlet
CH₂Cl₂=dichloromethane
d=doublet
dd=doublet of doublets
DBU=1,8-diazabicyclo[5.4.0]undec-7-ene
DCM=dichloromethane
DMAP=4-dimethylaminopyridine
DMF=N,N-dimethylformamide
DMSO=dimethyl sulfoxide
EA=ethyl acetate
EDCI=1-ethyl-3-(3-dimethylaminopropyl)carbodiimide hydrochloride
ESI=electrospray ionization
Et=ethyl
Et₃N=triethylamine
EtOAc=ethyl acetate
EtOH=ethyl alcohol
h=hours
HPLC=high-performance liquid chromatography
HOAc=acetic acid
LiOH=lithium hydroxide
m=multiplet
Me=methyl
MeCN=acetonitrile
MeOH=methyl alcohol
MgSO₄=magnesium sulfate
min=minutes
MS=mass spectroscopy
NaCl=sodium chloride
NaOH=sodium hydroxide
Na₂SO₄=sodium sulfate
NMI=N-methylimidazole
NMR=nuclear magnetic resonance spectroscopy
PE=petroleum ether
PG=protecting group
Ph=phenyl
q=quartet
rt=room temperature
s=singlet
t=triplet
TBME=t-butyl dimethyl ether
TFA=trifluoroacetic acid
THF=tetrahydrofuran
Ts=p-toluenesulfonyl (tosyl)
Tf=trifluoromethanesulfonyl

The Examples below provide illustrations of the conditions used for the preparation of the compounds of the present invention. The Examples provided are not intended to be limitations on the scope of the instant invention in any way, and they should not be so constructed. Those skilled in the art of nucleoside and nucleotide synthesis will readily appreciate that known variations of the conditions and processes of the following preparative procedures can be used to prepare these and other compounds of the present invention.

### Example 1

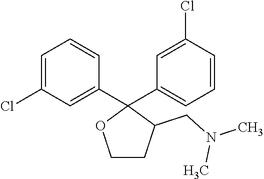 [SMILES: CN(C)CC1CCOC1(c1cccc(Cl)c1)c1cccc(Cl)c1]

### 1-(2,2-bis(3-chlorophenyl)tetrahydrofuran-3-yl)-N,N-dimethylmethanamine

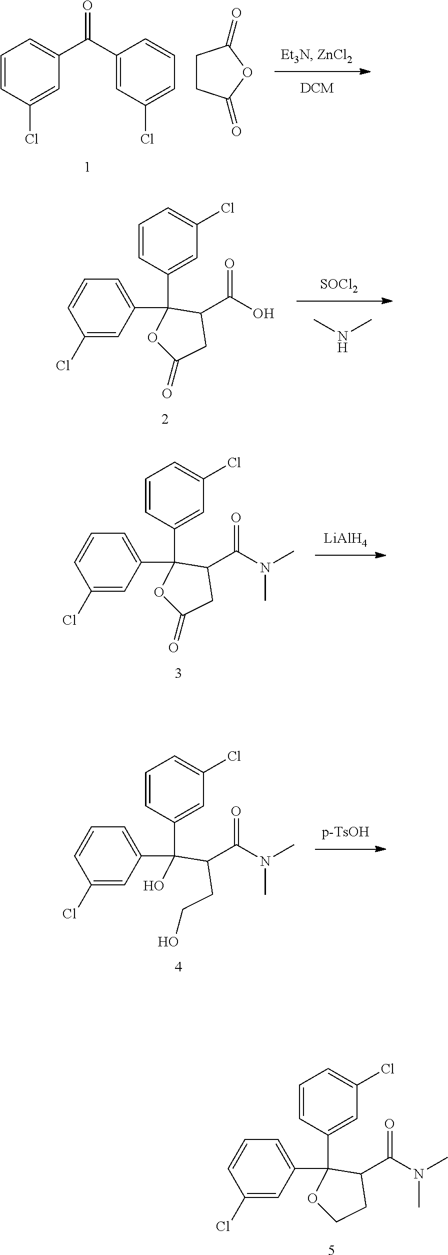

### Step 1: Intermediate 3

Zinc Chloride (1.36 g 10 mmol, dried in Situ with thionyl chloride), Succinic anhydride (750 mg, 7.5 mmol) and Bis-(3-chloro-phenyl)-methanone (1.25 g, 5 mmol) were suspended in dry DCM (6 ml). The stirred suspension was treated over a period of 10 minutes with triethylamine (1.39 ml, 10 mmol). The mixture became warm and nearly homogeneous and protected from moisture and was stirred gently for 20 to 24 hr. The solution was mixed with 2M HCl (10 ml) and ethyl acetate (10 ml). The organic phase was washed by brine three times, then the organic phase was extracted with aqueous NaHCO₃, then add Ethyl acetate again, followed by neutralized with 2M HCl to provide crude product to use the next step without further purification.

A mixture of crude 2 (0.6 g, 1.8 mmol) and 2 ml of thionyl chloride was heated at 60° C. for 1 h, the excess thionyl chloride was removed in vacuo and the resulting solid chloride was dissolved in dry THF (5 ml) in an ice bath, then a solution of dimethylamine in 2 ml of dry THF (300 mg, 6 mmol) was added dropwise with stirring for 3 h at ice bath temperature, and the dimethylamine HCl formed, then filtered off and washed with THF and concentrated to provide a crude solid product 3 with 500 mg. LCMS: for C₁₉H₁₇Cl₂NO₃, calculated 377.06, found 378.2 [M+H]⁺.

### Step 2: Intermediate 4

To a solution of compound 3 (500 mg, 1.32 mmol) in 5 ml of dry THF was added LiAlH4 (150 mg, 3.98 mmol), and the reaction mixture was refluxed for 10 h and then hydrolysed with water under low temperature. The solid was filtered off and washed with THF, the filtrate was dried by Na₂SO₄ and concentrated to provide 350 mg of product 4. LCMS: for C₁₉H₂₁Cl₂NO₃, calculated 381.09, found 382.21 [M+H]⁺.

### Step 3: 1-(2,2-bis(3-chlorophenyl)tetrahydrofuran-3-yl)-N,N-dimethylmethanamine (5)

A mixture of intermediate 4 (300 mg, 0.79 mmol) and P-toluenesulfonic acid (290 mg, 1.58 mmol) in dry 10 ml of dry toluene was refluxed for 5 h with continuous removal of water via a Dean-Stark trap. After treatment of the reaction mixture with 10 ml 1M NaOH solution, the organic layer was washed with water and evaporated to solid. The residue was purified by column chromatography on silica gel with (PE/EA=5/1 to 3/1). The solution was concentrated to provide 200 mg of the product 5. LCMS: for C₁₉H₁₉Cl₂NO, calculated 363.08, found 364.21 [M+H]⁺.

¹H NMR (400 MHz, DMSO-d₆) δ 7.46 (2H, s), 7.42 (2H, dd, J=7.5 Hz, 1.5 Hz), 7.35 (4H, m), 3.75 (2H, m), 2.56 (1H, brs), 2.28 (2H, m), 2.25 (6H, s), 1.79 (2H, m),

### Example 2

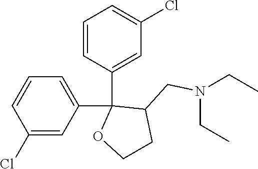 [SMILES: CCN(CC)CC1CCOC1(c1cccc(Cl)c1)c1cccc(Cl)c1]

### N-((2,2-bis(3-chlorophenyl)tetrahydrofuran-3-yl)methyl)-N-ethylethanamine

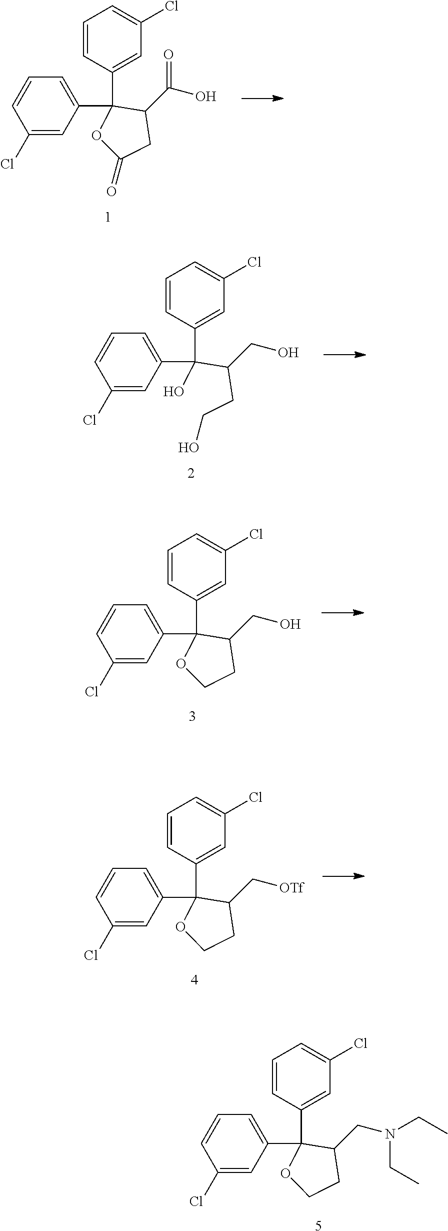

### Step 1: Intermediate 3

To a stirred dispersion of LiAlH₄ (1.20 g, 31.62 mmol) in anhydrous THF (40 ml) at 0° C., 2,2-bis(3-chlorophenyl)-5-oxotetrahydrofuran-3-carboxylic acid (1) (2.1 g, 6 mmol) was added portionwise and then the reaction mixture was heated to reflux for 10 h. The reaction was carefully decanted portionwise into water at 0° C., NaOH 40% aqueous solution (30 mL) was added and stirred for 30 min. The solid was filtered off and washed with THF, the filtrate was dried by Na₂SO₄ and concentrated to provide crude product 2. LCMS: for C₁₇H₁₈Cl₂O₃, calculated 340.06, found 341.21 [M+H]⁺.

A mixture of intermediate 3 (around 6 mmol) and P-toluenesulfonic acid (2.2 g, 12 mmol) in dry 80 ml of dry toluene was refluxed for 5 h with continuous removal of water via a Dean-Stark trap. After treatment of the reaction mixture with 10 ml 1M NaOH solution, the organic layer was washed with water and evaporated to solid. The residue was purified by column chromatography on silica gel with (PE/EA=5/1 to 3/1) to provide 1.5 g of the product 3. LCMS: for C₁₇H₁₆Cl₂O₂, calculated 322.05, found 323.11 [M+H]⁺.

### Step 2: N-((2,2-bis(3-chlorophenyl)tetrahydrofuran-3-yl)methyl)-N-ethylethanamine

To a stirred solution of compound 3 (674 mg, 1.77 mmol) in anhydrous DCM (23 ml) at −10 degree, 2,6-lutidine (0.31 ml, 2.65 mmol) and trifluoromethanesulfonic anhydride (0.435 ml, 2.65 mmol) were added dropwise. After stirring at −10 degrees for 1 h, the solvent was evaporated and EtOAc was added, the organic layer was washed very quickly with cold water and dried with Na₂SO₄. Evaporation of solvent in vacuo with out heating led to the crude triflate, which was dissolved in THF (5 mL) at 0 and a solution of Et₂NH in THF (10 mmol) was added with stirring. The reaction mixture was warmed to room temperature and stirred for overnight. The solvent was then evaporated, EtOAc was added, the organic layer was washed with water and evaporated in Vacuo. The residue was purified by flash column chromatography using a mixture of DCM:MeOH (95:5) as eluent and the product was obtained as a solid (640 mg).

LCMS: for C₂₁H₂₅Cl₂NO, calculated 377.13, found 378.21 [M+H]⁺.

¹H NMR (400 MHz, DMSO-d₆) δ 7.46 (2H, s), 7.42 (2H, dd, J=7.5 Hz, 1.5 Hz), 7.35 (4H, m), 3.75 (2H, m), 2.56 (1H, brs), 2.40 (4H, dd, J=8.0 Hz), 2.32 (2H, m), 1.81 (2H, m), 1.0 (6H, t, J=8.0 Hz)

### Example 3

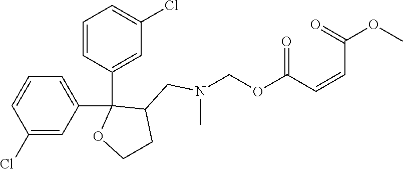 [SMILES: COC(=O)/C=C\C(=O)OCN(C)CC1CCOC1(c1cccc(Cl)c1)c1cccc(Cl)c1]

### (((2,2-bis(3-chlorophenyl)tetrahydrofuran-3-yl)methyl)(methyl)amino)methyl methyl maleate

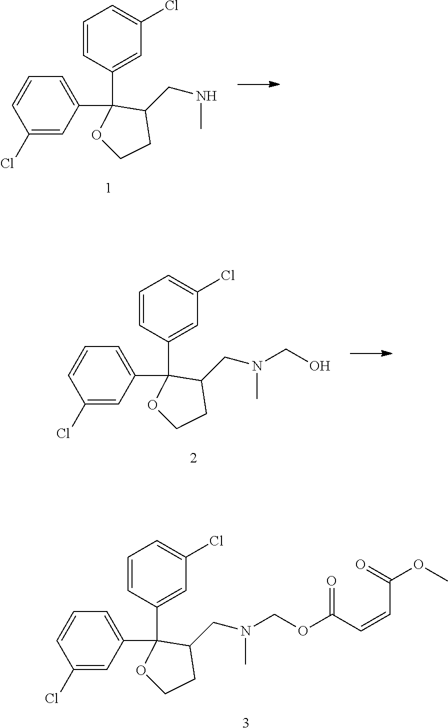

Step 1: A solution of the title compound 1 (268 mg, 0.8 mmmol) and formalin (1.2 ml, 37% aqueous solution) in acetonitrile (10 mL) was mixed in portions with sodium cyanoboron hydride (201 mg, 3.2 mmol) and stirred for 2 h at RT. Conc. acetic acid was added until a neutral reaction occurred and stirred for 45 h at RT. For work up the solvent was removed in a vacuum, the residue taken up in 2N NaOH (10 mL) and extracted with ether (3×20 mL). The organic solution was dried over Na₂SO₄ and concentrated to low volume in a vacuum. The remaining residue was as purified by flash chromatography with PE/EtOAc (3/1 to 1:1) to provide 250 mg of hydroxymethyl compound 2. LCMS: for C₁₉H₂₁Cl₂NO₂, calculated 365.09, found 366.21 [M+H]⁺.

### Step 2: (((2,2-bis(3-chlorophenyl)tetrahydrofuran-3-yl)methyl)(methyl)amino)methyl methyl maleate

To a solution of compound 2 in DMF (1 mmol) was add NaH (15 mmol) at 0° C. ice bath. The mixture was continued for 20 min and then added methyl (Z)-4-chloro-4-oxobut-2-enoate (2 mmol), stirred the reaction mixture at room temperature for 4 h. After completion of the reaction the mixture was cooled to 0° C. and added ice cold water dropwise. The reaction mixture was extracted with diethyl ether and the organic layer was dried over Na₂SO₄ and evaporated to get the crude product which was purified through column to get compound 2. LCMS: for C₂₄H₂₅Cl₂NO₅, calculated 477.11, found 478.21 [M+H]⁺.

¹H NMR (400 MHz, DMSO-d₆) δ 7.46 (2H, s), 7.42 (2H, dd, J=7.5 Hz, 1.5 Hz), 7.35 (4H, m), 6.49 (2H, s), 5.22 (2H, s), 4.06 (2H, dd, J=8.0 Hz), 3.75 (2H, m), 2.56 (1H, brs), 2.40 (2H, m), 2.26 (3H, s), 1.81 (2H, m), 1.21 (6H, t, J=8.0 Hz)

The compounds in Table 1 can be prepared by the same method as described in Examples 1, 2, 3.

TABLE 1
Compounds of Exmaples 4-172

| Ex. | Structure | MS[M+]⁺ | Name |
|---|---|---|---|
| 4 | 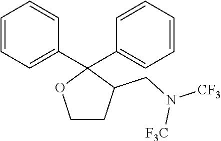 [SMILES: FC(F)(F)N(CC1CCOC1(c1ccccc1)c1ccccc1)C(F)(F)F] | 390.34 | N-((2,2-diphenyltetrahydrofuran-3- yl)methyl)-1,1,1-trifluoro-N- (trifluoromethyl)methanamine |
| 5 | 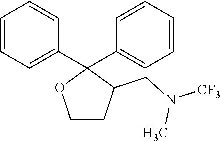 [SMILES: CN(CC1CCOC1(c1ccccc1)c1ccccc1)C(F)(F)F] | 336.27 | N-((2,2-diphenyltetrahydrofuran-3- yl)methyl)-1,1,1-trifluoro-N- methylmethanamine |
| 6 | 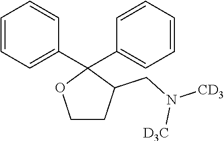 [SMILES: [2H]C([2H])([2H])N(CC1CCOC1(c1ccccc1)c1ccccc1)C([2H])([2H])[2H]] | 228.31 | N-((2,2-diphenyltetrahydrofuran-3- yl)methyl)-N-(methyl- d3)methanamine-d3 |
| 7 | 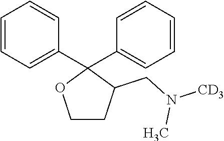 [SMILES: [2H]C([2H])([2H])N(C)CC1CCOC1(c1ccccc1)c1ccccc1] | 285.32 | N-((2,2-diphenyltetrahydrofuran-3- yl)methyl)-N- methylmethanamine-d3 |
| 8 | 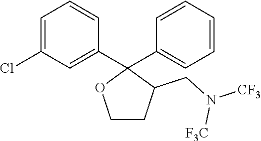 [SMILES: FC(F)(F)N(CC1CCOC1(c1ccccc1)c1cccc(Cl)c1)C(F)(F)F] | 425.18 | N-((2-(3-chlorophenyl)-2- phenyltetrahydrofuran-3-yl)methyl)- 1,1,1-trifluoro-N- (trifluoromethyl)methanamine |
| 9 | 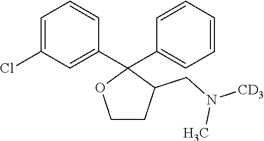 [SMILES: [2H]C([2H])([2H])N(C)CC1CCOC1(c1ccccc1)c1cccc(Cl)c1] | 320.26 | N-((2-(3-chlorophenyl)-2- phenyltetrahydrofuran-3-yl)methyl)- N-methylmethanamine-d3 |
| 10 | 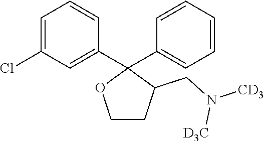 [SMILES: [2H]C([2H])([2H])N(CC1CCOC1(c1ccccc1)c1cccc(Cl)c1)C([2H])([2H])[2H]] | 323.28 | N-((2-(3-chlorophenyl)-2- phenyltetrahydrofuran-3-yl)methyl)- N-(methyl-d3)methanamine-d3 |
| 11 | 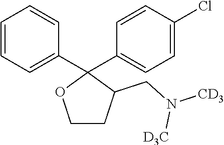 [SMILES: [2H]C([2H])([2H])N(CC1CCOC1(c1ccccc1)c1ccc(Cl)cc1)C([2H])([2H])[2H]] | 323.18 | N-((2-(4-chlorophenyl)-2- phenyltetrahydrofuran-3-yl)methyl)- N-(methyl-d3)methanamine-d3 |
| 12 | 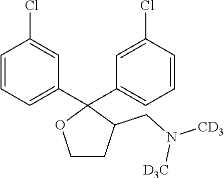 [SMILES: [2H]C([2H])([2H])N(CC1CCOC1(c1cccc(Cl)c1)c1cccc(Cl)c1)C([2H])([2H])[2H]] | 357.24 | N-((2,2-bis(3- chlorophenyl)tetrahydrofuran-3- yl)methyl)-N-(methyl- d3)methanamine-d3 |
| 13 | 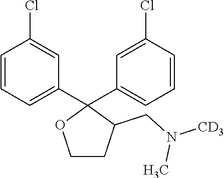 [SMILES: [2H]C([2H])([2H])N(C)CC1CCOC1(c1cccc(Cl)c1)c1cccc(Cl)c1] | 354.20 | N-((2,2-bis(3- chlorophenyl)tetrahydrofuran-3- yl)methyl)-N- methylmethanamine-d3 |
| 14 | 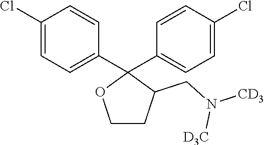 [SMILES: [2H]C([2H])([2H])N(CC1CCOC1(c1ccc(Cl)cc1)c1ccc(Cl)cc1)C([2H])([2H])[2H]] | 357.26 | N-((2,2-bis(4- chlorophenyl)tetrahydrofuran-3- yl)methyl)-N-(methyl- d3)methanamine-d3 |
| 15 | 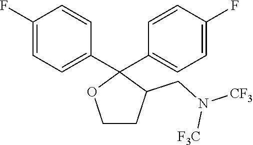 [SMILES: Fc1ccc(C2(c3ccc(F)cc3)OCCC2CN(C(F)(F)F)C(F)(F)F)cc1] | 426.20 | N-((2,2-bis(4- fluorophenyl)tetrahydrofuran-3- yl)methyl)-1,1,1-trifluoro-N- (trifluoromethyl)methanamine |
| 16 | 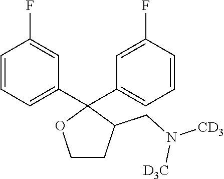 [SMILES: [2H]C([2H])([2H])N(CC1CCOC1(c1cccc(F)c1)c1cccc(F)c1)C([2H])([2H])[2H]] | 324.30 | N-((2,2-bis(3- fluorophenyl)tetrahydrofuran-3- yl)methyl)-N-(methyl- d3)methanamine-d3 |
| 17 | 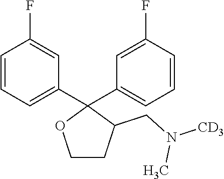 [SMILES: [2H]C([2H])([2H])N(C)CC1CCOC1(c1cccc(F)c1)c1cccc(F)c1] | 321.28 | N-((2,2-bis(3- fluorophenyl)tetrahydrofuran-3- yl)methyl)-N- methylmethanamine-d3 |
| 18 | 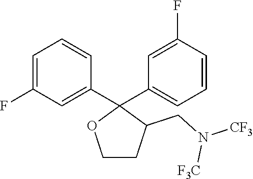 [SMILES: Fc1cccc(C2(c3cccc(F)c3)OCCC2CN(C(F)(F)F)C(F)(F)F)c1] | 426.20 | N-((2,2-bis(3- fluorophenyl)tetrahydrofuran-3- yl)methyl)-1,1,1-trifluoro-N- (trifluoromethyl)methanamine |
| 19 | 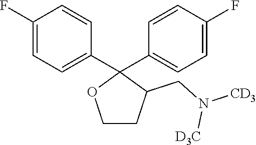 [SMILES: [2H]C([2H])([2H])N(CC1CCOC1(c1ccc(F)cc1)c1ccc(F)cc1)C([2H])([2H])[2H]] | 324.30 | N-((2,2-bis(4- fluorophenyl)tetrahydrofuran-3- yl)methyl)-N-(methyl- d3)methanamine-d3 |
| 20 | 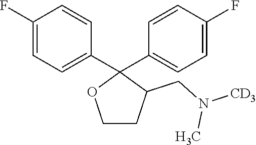 [SMILES: [2H]C([2H])([2H])N(C)CC1CCOC1(c1ccc(F)cc1)c1ccc(F)cc1] | 321.28 | N-((2,2-bis(4- fluorophenyl)tetrahydrofuran-3- yl)methyl)-N-methylmethanamine-d3 |
| 21 | 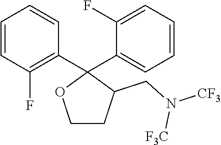 [SMILES: Fc1ccccc1C1(c2ccccc2F)OCCC1CN(C(F)(F)F)C(F)(F)F] | 426.11 | N-((2,2-bis(2- fluorophenyl)tetrahydrofuran-3- yl)methyl)-1,1,1-trifluoro-N- (trifluoromethyl)methanamine |
| 22 | 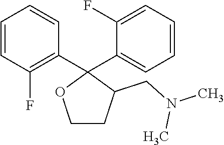 [SMILES: CN(C)CC1CCOC1(c1ccccc1F)c1ccccc1F] | 318.16 | 1-(2,2-bis(2- fluorophenyl)tetrahydrofuran-3-yl)- N,N-dimethylmethanamine |
| 23 | 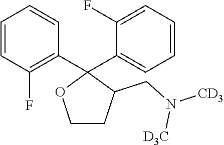 [SMILES: [2H]C([2H])([2H])N(CC1CCOC1(c1ccccc1F)c1ccccc1F)C([2H])([2H])[2H]] | 324.20 | N-((2,2-bis(2- fluorophenyl)tetrahydrofuran-3- yl)methyl)-N-(methyl- d3)methanamine-d3 |
| 24 | 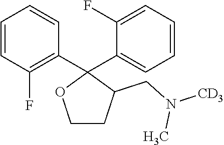 [SMILES: [2H]C([2H])([2H])N(C)CC1CCOC1(c1ccccc1F)c1ccccc1F] | 321.18 | N-((2,2-bis(2- fluorophenyl)tetrahydrofuran-3- yl)methyl)-N- methylmethanamine-d3 |
| 25 | 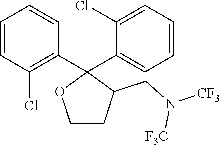 [SMILES: FC(F)(F)N(CC1CCOC1(c1ccccc1Cl)c1ccccc1Cl)C(F)(F)F] | 459.04 | N-((2,2-bis(2- chlorophenyl)tetrahydrofuran-3- yl)methyl)-1,1,1-trifluoro-N- (trifluoromethyl)methanamine |
| 26 | 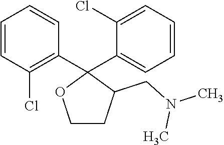 [SMILES: CN(C)CC1CCOC1(c1ccccc1Cl)c1ccccc1Cl] | 351.10 | 1-(2,2-bis(2- chlorophenyl)tetrahydrofuran-3-yl)- N,N-dimethylmethanamine |
| 27 | 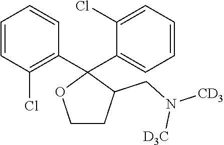 [SMILES: [2H]C([2H])([2H])N(CC1CCOC1(c1ccccc1Cl)c1ccccc1Cl)C([2H])([2H])[2H]] | 357.13 | N-((2,2-bis(2- chlorophenyl)tetrahydrofuran-3- yl)methyl)-N-(methyl- d3)methanamine-d3 |
| 28 | 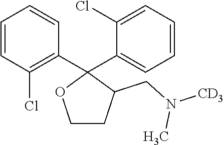 [SMILES: [2H]C([2H])([2H])N(C)CC1CCOC1(c1ccccc1Cl)c1ccccc1Cl] | 354.12 | N-((2,2-bis(2- chlorophenyl)tetrahydrofuran-3- yl)methyl)-N-methylmethanamine-d3 |
| 29 | 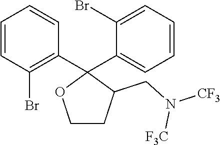 [SMILES: FC(F)(F)N(CC1CCOC1(c1ccccc1Br)c1ccccc1Br)C(F)(F)F] | 548.94 | N-((2,2-bis(2- bromophenyl)tetrahydrofuran-3- yl)methyl)-1,1,1-trifluoro-N- (trifluoromethyl)methanamine |
| 30 | 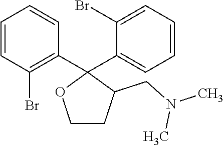 [SMILES: CN(C)CC1CCOC1(c1ccccc1Br)c1ccccc1Br] | 440.99 | 1-(2,2-bis(2- bromophenyl)tetrahydrofuran-3-yl)- N,N-dimethylmethanamine |
| 31 | 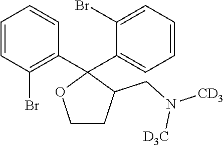 [SMILES: [2H]C([2H])([2H])N(CC1CCOC1(c1ccccc1Br)c1ccccc1Br)C([2H])([2H])[2H]] | 447.03 | N-((2,2-bis(2- bromophenyl)tetrahydrofuran-3- yl)methyl)-N-(methyl- d3)methanamine-d3 |
| 32 | 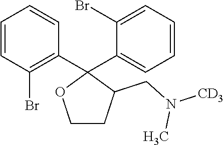 [SMILES: [2H]C([2H])([2H])N(C)CC1CCOC1(c1ccccc1Br)c1ccccc1Br] | 444.01 | N-((2,2-bis(2- bromophenyl)tetrahydrofuran-3- yl)methyl)-N- methylmethanamine-d3 |
| 33 | 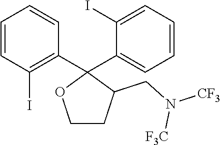 [SMILES: FC(F)(F)N(CC1CCOC1(c1ccccc1I)c1ccccc1I)C(F)(F)F] | 641.92 | N-((2,2-bis(2- iodophenyl)tetrahydrofuran-3- yl)methyl)-1,1,1-trifluoro-N- (trifluoromethyl)methanamine |
| 34 | 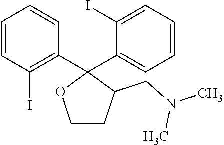 [SMILES: CN(C)CC1CCOC1(c1ccccc1I)c1ccccc1I] | 533.97 | 1-(2,2-bis(2- iodophenyl)tetrahydrofuran-3-yl)- N,N-dimethylmethanamine |
| 35 | 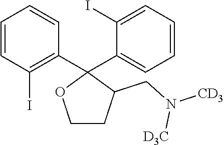 [SMILES: [2H]C([2H])([2H])N(CC1CCOC1(c1ccccc1I)c1ccccc1I)C([2H])([2H])[2H]] | 541.02 | N-((2,2-bis(2- iodophenyl)tetrahydrofuran-3- yl)methyl)-N-(methyl- d3)methanamine-d3 |
| 36 | 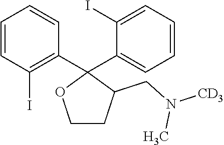 [SMILES: [2H]C([2H])([2H])N(C)CC1CCOC1(c1ccccc1I)c1ccccc1I] | 536.99 | N-((2,2-bis(2- iodophenyl)tetrahydrofuran-3- yl)methyl)-N- methylmethanamine-d3 |
| 37 | 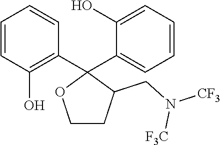 [SMILES: Oc1ccccc1C1(c2ccccc2O)OCCC1CN(C(F)(F)F)C(F)(F)F] | 422.11 | 2,2′-(3- ((bis(trifluoromethyl)amino)methyl) tetrahydrofuran-2,2-diyl)diphenol |
| 38 | 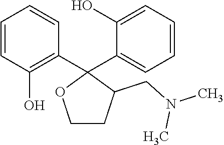 [SMILES: CN(C)CC1CCOC1(c1ccccc1O)c1ccccc1O] | 314.17 | 2,2′-(3- ((dimethylamino)methyl) tetrahydrofuran-2,2-diyl)diphenol |
| 39 | 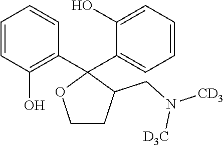 [SMILES: [2H]C([2H])([2H])N(CC1CCOC1(c1ccccc1O)c1ccccc1O)C([2H])([2H])[2H]] | 320.21 | 2,2′-(3-((bis(methyl- d3)amino)methyl)tetrahydrofuran- 2,2-diyl)diphenol |
| 40 | 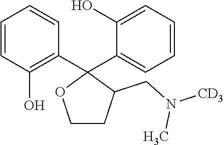 [SMILES: [2H]C([2H])([2H])N(C)CC1CCOC1(c1ccccc1O)c1ccccc1O] | 317.19 | 2,2′-(3-((methyl(methyl- d3)amino)methyl)tetrahydrofuran- 2,2-diyl)diphenol |
| 41 | 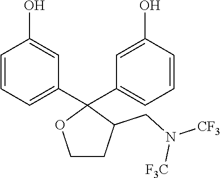 [SMILES: Oc1cccc(C2(c3cccc(O)c3)OCCC2CN(C(F)(F)F)C(F)(F)F)c1] | 422.11 | 3,3′-(3- ((bis(trifluoromethyl)amino)methyl) tetrahydrofuran-2,2-diyl)diphenol |
| 42 | 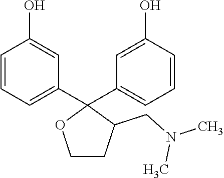 [SMILES: CN(C)CC1CCOC1(c1cccc(O)c1)c1cccc(O)c1] | 314.17 | 3,3′-(3- ((dimethylamino)methyl) tetrahydrofuran-2,2-diyl)diphenol |
| 43 | 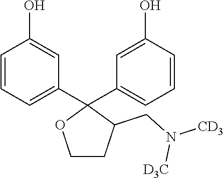 [SMILES: [2H]C([2H])([2H])N(CC1CCOC1(c1cccc(O)c1)c1cccc(O)c1)C([2H])([2H])[2H]] | 320.21 | 3,3′-(3-((bis(methyl- d3)amino)methyl)tetrahydrofuran- 2,2-diyl)diphenol |
| 44 | 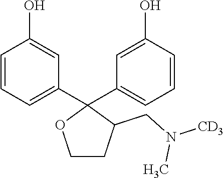 [SMILES: [2H]C([2H])([2H])N(C)CC1CCOC1(c1cccc(O)c1)c1cccc(O)c1] | 317.19 | 3,3′-(3-((methyl(methyl- d3)amino)methyl)tetrahydrofuran- 2,2-diyl)diphenol |
| 45 | 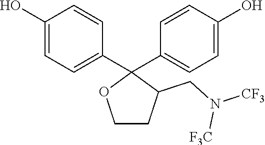 [SMILES: Oc1ccc(C2(c3ccc(O)cc3)OCCC2CN(C(F)(F)F)C(F)(F)F)cc1] | 422.11 | 4,4′-(3- ((bis(trifluoromethyl)amino)methyl) tetrahydrofuran-2,2-diyl)diphenol |
| 46 | 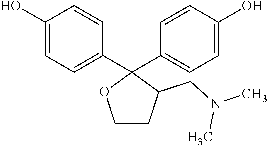 [SMILES: CN(C)CC1CCOC1(c1ccc(O)cc1)c1ccc(O)cc1] | 314.17 | 4,4′-(3- ((dimethylamino)methyl) tetrahydrofuran-2,2-diyl)diphenol |
| 47 | 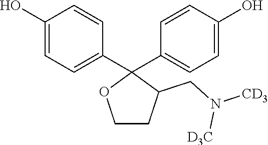 [SMILES: [2H]C([2H])([2H])N(CC1CCOC1(c1ccc(O)cc1)c1ccc(O)cc1)C([2H])([2H])[2H]] | 320.21 | 4,4′-(3-((bis(methyl- d3)amino)methyl)tetrahydrofuran- 2,2-diyl)diphenol |
| 48 | 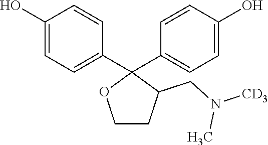 [SMILES: [2H]C([2H])([2H])N(C)CC1CCOC1(c1ccc(O)cc1)c1ccc(O)cc1] | 317.19 | 4,4′-(3-((methyl(methyl- d3)amino)methyl)tetrahydrofuran- 2,2-diyl)diphenol |
| 49 | 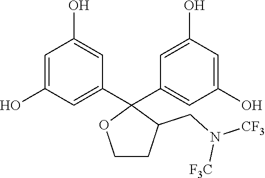 [SMILES: Oc1cc(O)cc(C2(c3cc(O)cc(O)c3)OCCC2CN(C(F)(F)F)C(F)(F)F)c1] | 454.10 | 5,5′-(3- ((bis(trifluoromethyl)amino)methyl) tetrahydrofuran-2,2-diyl)bis (benzene-1,3-diol) |
| 50 | 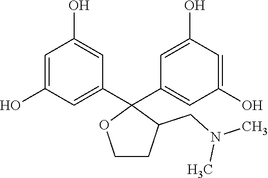 [SMILES: CN(C)CC1CCOC1(c1cc(O)cc(O)c1)c1cc(O)cc(O)c1] | 346.16 | 5,5′-(3- ((dimethylamino)methyl) tetrahydrofuran-2,2-diyl) bis(benzene-1,3-diol) |
| 51 | 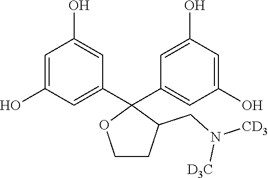 [SMILES: [2H]C([2H])([2H])N(CC1CCOC1(c1cc(O)cc(O)c1)c1cc(O)cc(O)c1)C([2H])([2H])[2H]] | 352.20 | 5,5′-(3-((bis(methyl- d3)amino)methyl)tetrahydrofuran- 2,2-diyl)bis(benzene-1,3-diol) |
| 52 | 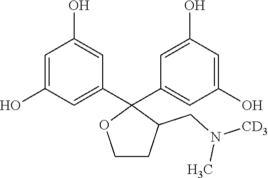 [SMILES: [2H]C([2H])([2H])N(C)CC1CCOC1(c1cc(O)cc(O)c1)c1cc(O)cc(O)c1] | 349.18 | 5,5′-(3-((methyl(methyl- d3)amino)methyl)tetrahydrofuran- 2,2-diyl)bis(benzene-1,3-diol) |
| 53 | 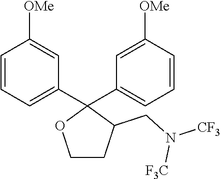 [SMILES: COc1cccc(C2(c3cccc(OC)c3)OCCC2CN(C(F)(F)F)C(F)(F)F)c1] | 450.15 | N-((2,2-bis(3- methoxyphenyl)tetrahydrofuran-3- yl)methyl)-1,1,1-trifluoro-N- (trifluoromethyl)methanamine |
| 54 | 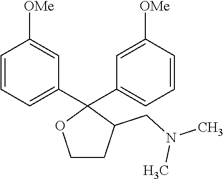 [SMILES: COc1cccc(C2(c3cccc(OC)c3)OCCC2CN(C)C)c1] | 342.20 | 1-(2,2-bis(3- methoxyphenyl)tetrahydrofuran-3- yl)-N,N-dimethylmethanamine |
| 55 | 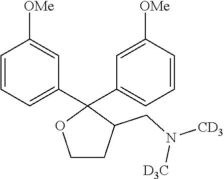 [SMILES: [2H]C([2H])([2H])N(CC1CCOC1(c1cccc(OC)c1)c1cccc(OC)c1)C([2H])([2H])[2H]] | 348.24 | N-((2,2-bis(3- methoxyphenyl)tetrahydrofuran-3- yl)methyl)-N-(methyl- d3)methanamine-d3 |
| 56 | 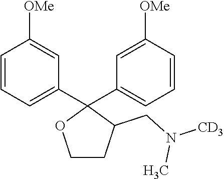 [SMILES: [2H]C([2H])([2H])N(C)CC1CCOC1(c1cccc(OC)c1)c1cccc(OC)c1] | 345.22 | N-((2,2-bis(3- methoxyphenyl)tetrahydrofuran-3- yl)methyl)-N- methylmethanamine-d3 |
| 57 | 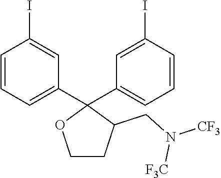 [SMILES: FC(F)(F)N(CC1CCOC1(c1cccc(I)c1)c1cccc(I)c1)C(F)(F)F] | 641.92 | N-((2,2-bis(3- iodophenyl)tetrahydrofuran-3- yl)methyl)-1,1,1-trifluoro-N- (trifluoromethyl)methanamine |
| 58 | 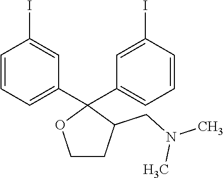 [SMILES: CN(C)CC1CCOC1(c1cccc(I)c1)c1cccc(I)c1] | 533.97 | 1-(2,2-bis(3- iodophenyl)tetrahydrofuran-3-yl)- N,N-dimethylmethanamine |
| 59 | 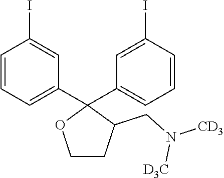 [SMILES: [2H]C([2H])([2H])N(CC1CCOC1(c1cccc(I)c1)c1cccc(I)c1)C([2H])([2H])[2H]] | 540.01 | N-((2,2-bis(3- iodophenyl)tetrahydrofuran-3- yl)methyl)-N-(methyl- d3)methanamine-d3 |
| 60 | 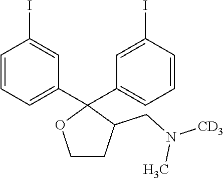 [SMILES: [2H]C([2H])([2H])N(C)CC1CCOC1(c1cccc(I)c1)c1cccc(I)c1] | 536.99 | N-((2,2-bis(3- iodophenyl)tetrahydrofuran-3- yl)methyl)-N- methylmethanamine-d3 |
| 61 | 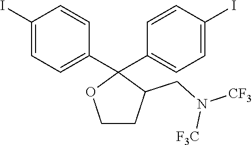 [SMILES: FC(F)(F)N(CC1CCOC1(c1ccc(I)cc1)c1ccc(I)cc1)C(F)(F)F] | 641.92 | N-((2,2-bis(4- iodophenyl)tetrahydrofuran-3- yl)methyl)-1,1,1-trifluoro-N- (trifluoromethyl)methanamine |
| 62 | 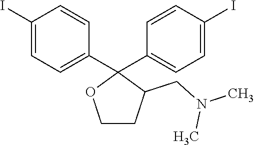 [SMILES: CN(C)CC1CCOC1(c1ccc(I)cc1)c1ccc(I)cc1] | 533.97 | 1-(2,2-bis(4- iodophenyl)tetrahydrofuran-3-yl)- N,N-dimethylmethanamine |
| 63 | 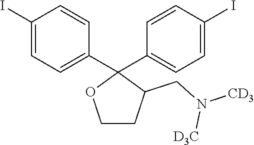 [SMILES: [2H]C([2H])([2H])N(CC1CCOC1(c1ccc(I)cc1)c1ccc(I)cc1)C([2H])([2H])[2H]] | 540.01 | N-((2,2-bis(4- iodophenyl)tetrahydrofuran-3- yl)methyl)-N-(methyl- d3)methanamine-d3 |
| 64 | 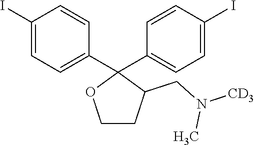 [SMILES: [2H]C([2H])([2H])N(C)CC1CCOC1(c1ccc(I)cc1)c1ccc(I)cc1] | 536.99 | N-((2,2-bis(4- iodophenyl)tetrahydrofuran-3- yl)methyl)-N- methylmethanamine-d3 |
| 65 | 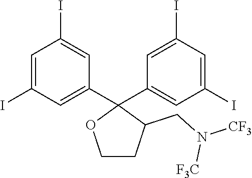 [SMILES: FC(F)(F)N(CC1CCOC1(c1cc(I)cc(I)c1)c1cc(I)cc(I)c1)C(F)(F)F] | 893.71 | N-((2,2-bis(3,5- diiodophenyl)tetrahydrofuran-3- yl)methyl)-1,1,1-trifluoro-N- (trifluoromethyl)methanamine |
| 66 | 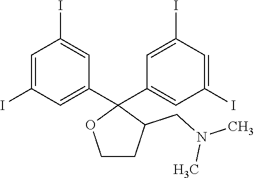 [SMILES: CN(C)CC1CCOC1(c1cc(I)cc(I)c1)c1cc(I)cc(I)c1] | 785.77 | 1-(2,2-bis(3,5- diiodophenyl)tetrahydrofuran-3-yl)- N,N-dimethylmethanamine |
| 67 | 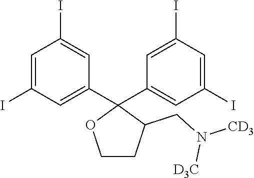 [SMILES: [2H]C([2H])([2H])N(CC1CCOC1(c1cc(I)cc(I)c1)c1cc(I)cc(I)c1)C([2H])([2H])[2H]] | 791.81 | N-((2,2-bis(3,5- diiodophenyl)tetrahydrofuran-3- yl)methyl)-N-(methyl- d3)methanamine-d3 |
| 68 | 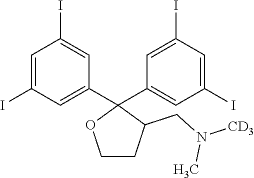 [SMILES: [2H]C([2H])([2H])N(C)CC1CCOC1(c1cc(I)cc(I)c1)c1cc(I)cc(I)c1] | 788.79 | N-((2,2-bis(3,5- diiodophenyl)tetrahydrofuran-3- yl)methyl)-N- methylmethanamine-d3 |
| 69 | 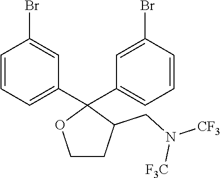 [SMILES: FC(F)(F)N(CC1CCOC1(c1cccc(Br)c1)c1cccc(Br)c1)C(F)(F)F] | 548.94 | N-((2,2-bis(3- bromophenyl)tetrahydrofuran-3- yl)methyl)-1,1,1-trifluoro-N- (trifluoromethyl)methanamine |
| 70 | 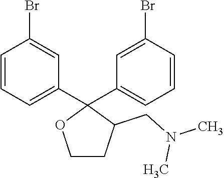 [SMILES: CN(C)CC1CCOC1(c1cccc(Br)c1)c1cccc(Br)c1] | 440.99 | 1-(2,2-bis(3- bromophenyl)tetrahydrofuran-3-yl)- N,N-dimethylmethanamine |
| 71 | 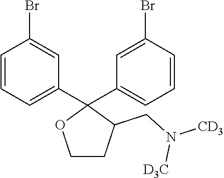 [SMILES: [2H]C([2H])([2H])N(CC1CCOC1(c1cccc(Br)c1)c1cccc(Br)c1)C([2H])([2H])[2H]] | 447.03 | N-((2,2-bis(3- bromophenyl)tetrahydrofuran-3- yl)methyl)-N-(methyl- d3)methanamine-d3 |
| 72 | 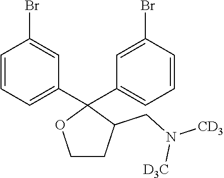 [SMILES: [2H]C([2H])([2H])N(CC1CCOC1(c1cccc(Br)c1)c1cccc(Br)c1)C([2H])([2H])[2H]] | 444.01 | N-((2,2-bis(3- bromophenyl)tetrahydrofuran-3- yl)methyl)-N- methylmethanamine-d3 |
| 73 | 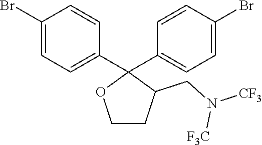 [SMILES: FC(F)(F)N(CC1CCOC1(c1ccc(Br)cc1)c1ccc(Br)cc1)C(F)(F)F] | 548.94 | N-((2,2-bis(4- bromophenyl)tetrahydrofuran-3- yl)methyl)-1,1,1-trifluoro-N- (trifluoromethyl)methanamine |
| 74 | 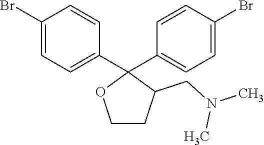 [SMILES: CN(C)CC1CCOC1(c1ccc(Br)cc1)c1ccc(Br)cc1] | 440.99 | 1-(2,2-bis(4- bromophenyl)tetrahydrofuran-3-yl)- N,N-dimethylmethanamine |
| 75 | 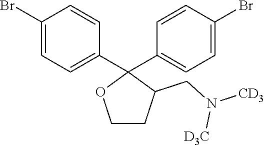 [SMILES: [2H]C([2H])([2H])N(CC1CCOC1(c1ccc(Br)cc1)c1ccc(Br)cc1)C([2H])([2H])[2H]] | 447.03 | N-((2,2-bis(4- bromophenyl)tetrahydrofuran-3- yl)methyl)-N-(methyl- d3)methanamine-d3 |
| 76 | 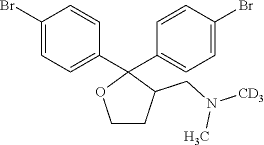 [SMILES: [2H]C([2H])([2H])N(C)CC1CCOC1(c1ccc(Br)cc1)c1ccc(Br)cc1] | 444.01 | N-((2,2-bis(4- bromophenyl)tetrahydrofuran-3- yl)methyl)-N-methylmethanamine-d3 |
| 77 | 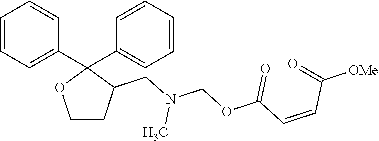 [SMILES: COC(=O)/C=C\C(=O)OCN(C)CC1CCOC1(c1ccccc1)c1ccccc1] | 410.23 | (((2,2-diphenyltetrahydrofuran-3- yl)methyl)(methyl)amino)methyl methyl maleate |
| 78 | 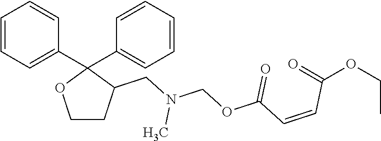 [SMILES: CCOC(=O)/C=C\C(=O)OCN(C)CC1CCOC1(c1ccccc1)c1ccccc1] | 424.30 | (((2,2-diphenyltetrahydrofuran-3- yl)methyl)(methyl)amino)methyl ethyl maleate |
| 79 | 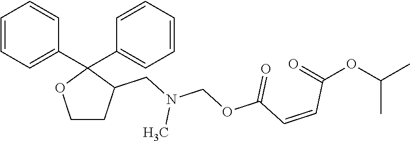 [SMILES: CC(C)OC(=O)/C=C\C(=O)OCN(C)CC1CCOC1(c1ccccc1)c1ccccc1] | 438.64 | (((2,2-diphenyltetrahydrofuran-3- yl)methyl)(methyl)amino)methyl isopropyl maleate |
| 80 | 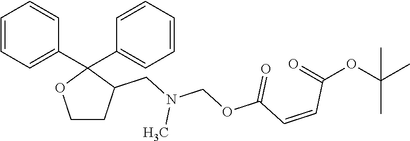 [SMILES: CN(COC(=O)/C=C\C(=O)OC(C)(C)C)CC1CCOC1(c1ccccc1)c1ccccc1] | 452.34 | tert-butyl ((((2,2- diphenyltetrahydrofuran-3- yl)methyl)(methyl)amino)methyl) maleate |
| 81 | 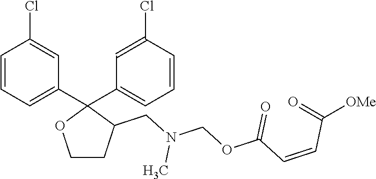 [SMILES: COC(=O)/C=C\C(=O)OCN(C)CC1CCOC1(c1cccc(Cl)c1)c1cccc(Cl)c1] | 479.21 | (((2,2-bis(3- chlorophenyl)tetrahydrofuran-3- yl)methyl)(methyl)amino)methyl methyl maleate |
| 82 | 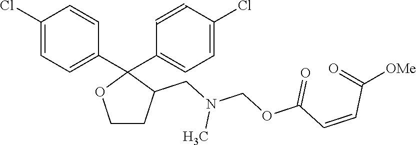 [SMILES: COC(=O)/C=C\C(=O)OCN(C)CC1CCOC1(c1ccc(Cl)cc1)c1ccc(Cl)cc1] | 479.15 | (((2,2-bis(4- chlorophenyl)tetrahydrofuran-3- yl)methyl)(methyl)amino)methyl methyl maleate |
| 83 | 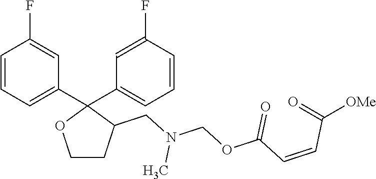 [SMILES: COC(=O)/C=C\C(=O)OCN(C)CC1CCOC1(c1cccc(F)c1)c1cccc(F)c1] | 446.27 | (((2,2-bis(3- fluorophenyl)tetrahydrofuran-3- yl)methyl)(methyl)amino)methyl methyl maleate |
| 84 | 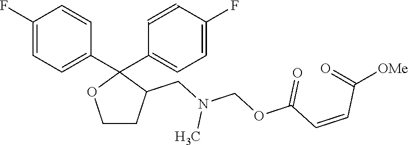 [SMILES: COC(=O)/C=C\C(=O)OCN(C)CC1CCOC1(c1ccc(F)cc1)c1ccc(F)cc1] | 446.27 | (((2,2-bis(4- fluorophenyl)tetrahydrofuran-3- yl)methyl)(methyl)amino)methyl methyl maleate |
| 85 | 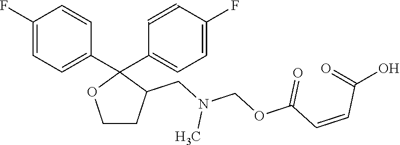 [SMILES: CN(COC(=O)/C=C\C(=O)O)CC1CCOC1(c1ccc(F)cc1)c1ccc(F)cc1] | 432.25 | (Z)-4-((((2,2-bis(4- fluorophenyl)tetrahydrofuran-3- yl)methyl)(methyl)amino)methoxy)- 4-oxobut-2-enoic acid |
| 86 | 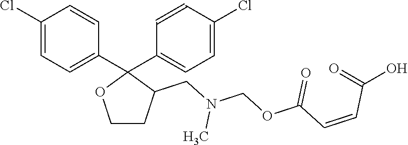 [SMILES: CN(COC(=O)/C=C\C(=O)O)CC1CCOC1(c1ccc(Cl)cc1)c1ccc(Cl)cc1] | 465.15 | (Z)-4-((((2,2-bis(4- chlorophenyl)tetrahydrofuran-3- yl)methyl)(methyl)amino)methoxy)- 4-oxobut-2-enoic acid |
| 87 | 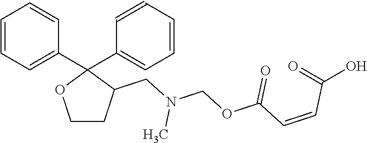 [SMILES: CN(COC(=O)/C=C\C(=O)O)CC1CCOC1(c1ccccc1)c1ccccc1] | 396.27 | (Z)-4-((((2,2- diphenyltetrahydrofuran-3- yl)methyl)(methyl)amino)methoxy)- 4-oxobut-2-enoic acid |
| 88 | 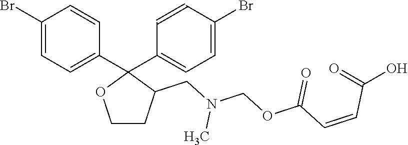 [SMILES: CN(COC(=O)/C=C\C(=O)O)CC1CCOC1(c1ccc(Br)cc1)c1ccc(Br)cc1] | 554.99 | (Z)-4-((((2,2-bis(4- bromophenyl)tetrahydrofuran-3- yl)methyl)(methyl)amino)methoxy)- 4-oxobut-2-enoic acid |
| 89 | 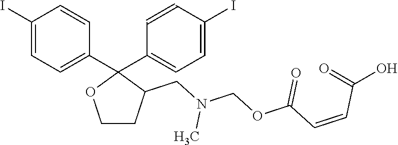 [SMILES: CN(COC(=O)/C=C\C(=O)O)CC1CCOC1(c1ccc(I)cc1)c1ccc(I)cc1] | 647.97 | (Z)-4-((((2,2-bis(4- iodophenyl)tetrahydrofuran-3- yl)methyl)(methyl)amino)methoxy)- 4-oxobut-2-enoic acid |
| 90 | 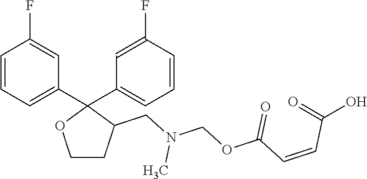 [SMILES: CN(COC(=O)/C=C\C(=O)O)CC1CCOC1(c1cccc(F)c1)c1cccc(F)c1] | 432.16 | (Z)-4-((((2,2-bis(3- fluorophenyl)tetrahydrofuran-3- yl)methyl)(methyl)amino)methoxy)- 4-oxobut-2-enoic acid |
| 91 | 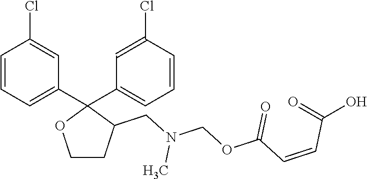 [SMILES: CN(COC(=O)/C=C\C(=O)O)CC1CCOC1(c1cccc(Cl)c1)c1cccc(Cl)c1] | 465.15 | (Z)-4-((((2,2-bis(3- chlorophenyl)tetrahydrofuran-3- yl)methyl)(methyl)amino)methoxy)- 4-oxobut-2-enoic acid |
| 92 | 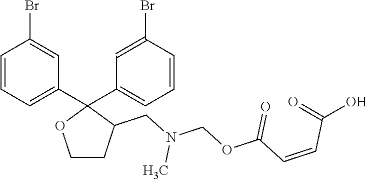 [SMILES: CN(COC(=O)/C=C\C(=O)O)CC1CCOC1(c1cccc(Br)c1)c1cccc(Br)c1] | 554.99 | (Z)-4-((((2,2-bis(3- bromophenyl)tetrahydrofuran-3- yl)methyl)(methyl)amino)methoxy)- 4-oxobut-2-enoic acid |
| 93 | 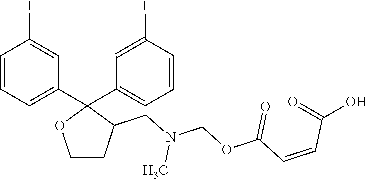 [SMILES: CN(COC(=O)/C=C\C(=O)O)CC1CCOC1(c1cccc(I)c1)c1cccc(I)c1] | 647.97 | (Z)-4-((((2,2-bis(3- iodophenyl)tetrahydrofuran-3- yl)methyl)(methyl)amino)methoxy)- 4-oxobut-2-enoic acid |
| 94 | 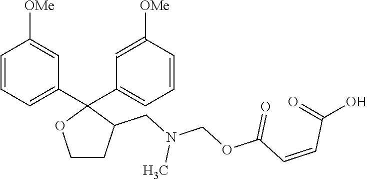 [SMILES: COc1cccc(C2(c3cccc(OC)c3)OCCC2CN(C)COC(=O)/C=C\C(=O)O)c1] | 456.20 | (Z)-4-((((2,2-bis(3- methoxyphenyl)tetrahydrofuran-3- yl)methyl)(methyl)amino)methoxy)- 4-oxobut-2-enoic acid |
| 95 | 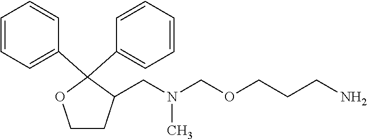 [SMILES: CN(COCCCN)CC1CCOC1(c1ccccc1)c1ccccc1] | 356.35 | 1-(3-(14-azanyl)propoxy)-N-((2,2- diphenyltetrahydrofuran-3- yl)methyl)-N- methylmethanamine |
| 96 | 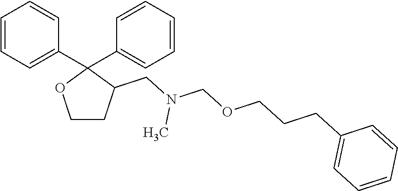 [SMILES: CN(COCCCc1ccccc1)CC1CCOC1(c1ccccc1)c1ccccc1] | 416.35 | 1-(2,2-diphenyltetrahydrofuran-3-yl)- N-methyl-N-((3- phenylpropoxy)methyl) methanamine |
| 97 | 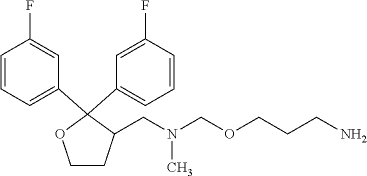 [SMILES: CN(COCCCN)CC1CCOC1(c1cccc(F)c1)c1cccc(F)c1] | 392.33 | 1-(3-(14-azanyl)propoxy)-N-((2,2- bis(4-fluorophenyl)tetrahydrofuran- 3-yl)methyl)-N- methylmethanamine |
| 98 | 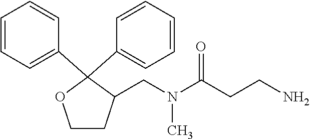 [SMILES: CN(CC1CCOC1(c1ccccc1)c1ccccc1)C(=O)CCN] | 340.25 | 3-(14-azanyl)-N-((2,2- diphenyltetrahydrofuran-3- yl)methyl)-N-methylpropanamide |
| 99 | 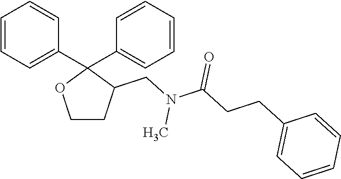 [SMILES: CN(CC1CCOC1(c1ccccc1)c1ccccc1)C(=O)CCc1ccccc1] | 400.32 | N-((2,2-diphenyltetrahydrofuran-3- yl)methyl)-N-methyl-3- phenylpropanamide |
| 100 | 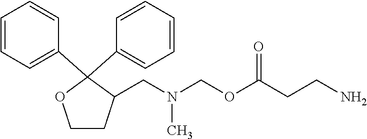 [SMILES: CN(COC(=O)CCN)CC1CCOC1(c1ccccc1)c1ccccc1] | 370.25 | (((2,2-diphenyltetrahydrofuran-3- yl)methyl)(methyl)amino)methyl 3-(l4-azanyl)propanoate |
| 101 | 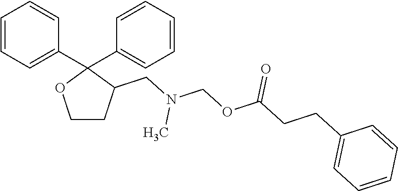 [SMILES: CN(COC(=O)CCc1ccccc1)CC1CCOC1(c1ccccc1)c1ccccc1] | 430.33 | (((2,2-diphenyltetrahydrofuran-3- yl)methyl)(methyl)amino)methyl 3-phenylpropanoate |
| 102 | 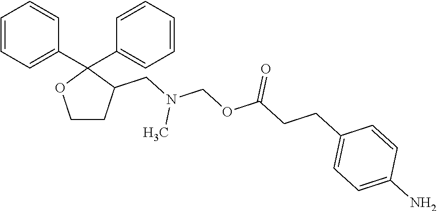 [SMILES: CN(COC(=O)CCc1ccc(N)cc1)CC1CCOC1(c1ccccc1)c1ccccc1] | 445.34 | (((2,2-diphenyltetrahydrofuran-3- yl)methyl)(methyl)amino)methyl 3-(4-aminophenyl)propanoate |
| 103 | 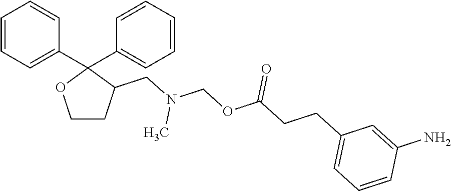 [SMILES: CN(COC(=O)CCc1cccc(N)c1)CC1CCOC1(c1ccccc1)c1ccccc1] | 445.25 | (((2,2-diphenyltetrahydrofuran-3- yl)methyl)(methyl)amino)methyl 3-(3-aminophenyl)propanoate |
| 104 | 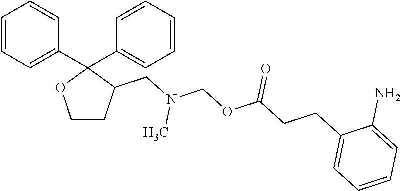 [SMILES: CN(COC(=O)CCc1ccccc1N)CC1CCOC1(c1ccccc1)c1ccccc1] | 445.26 | (((2,2-diphenyltetrahydrofuran-3- yl)methyl)(methyl)amino)methyl 3-(2-aminophenyl)propanoate |
| 105 | 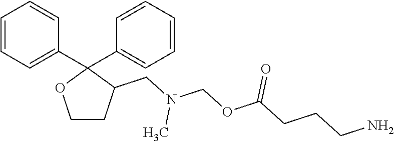 [SMILES: CN(COC(=O)CCCN)CC1CCOC1(c1ccccc1)c1ccccc1] | 355.29 | (((2,2-diphenyltetrahydrofuran-3- yl)methyl)(methyl)amino)methyl glycinate |
| 106 | 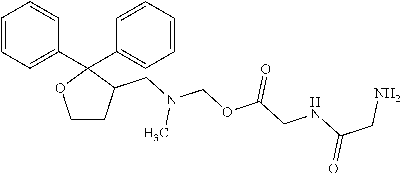 [SMILES: CN(COC(=O)CNC(=O)CN)CC1CCOC1(c1ccccc1)c1ccccc1] | 412.22 | (((2,2-diphenyltetrahydrofuran-3- yl)methyl)(methyl)amino)methyl glycylglycinate |
| 107 | 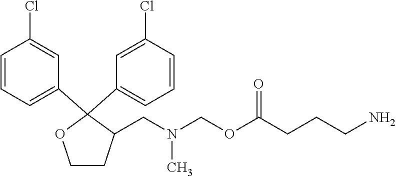 [SMILES: CN(COC(=O)CCCN)CC1CCOC1(c1cccc(Cl)c1)c1cccc(Cl)c1] | 452.14 | (((2,2-bis(3- chlorophenyl)tetrahydrofuran-3- yl)methyl)(methyl)amino)methyl 4-aminobutanoate |
| 108 | 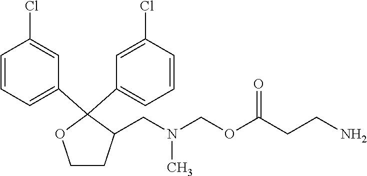 [SMILES: CN(COC(=O)CCN)CC1CCOC1(c1cccc(Cl)c1)c1cccc(Cl)c1] | 439.24 | (((2,2-bis(3- chlorophenyl)tetrahydrofuran-3- yl)methyl)(methyl)amino)methyl 3-(14-azanyl)propanoate |
| 109 | 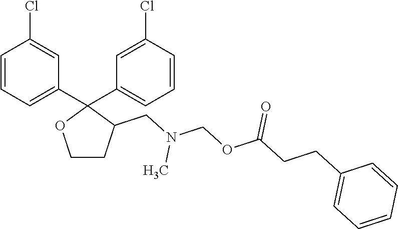 [SMILES: CN(COC(=O)CCc1ccccc1)CC1CCOC1(c1cccc(Cl)c1)c1cccc(Cl)c1] | 499.17 | (((2,2-bis(3- chlorophenyl)tetrahydrofuran-3- yl)methyl)(methyl)amino)methyl 3-phenylpropanoate |
| 110 | 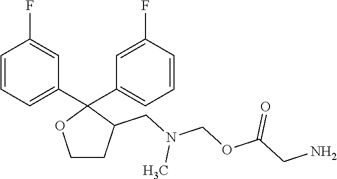 [SMILES: CN(COC(=O)CN)CC1CCOC1(c1cccc(F)c1)c1cccc(F)c1] | 391.28 | (((2,2-bis(3- fluorophenyl)tetrahydrofuran-3- yl)methyl)(methyl)amino)methyl glycinate |
| 111 | 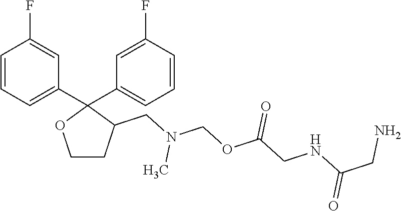 [SMILES: CN(COC(=O)CNC(=O)CN)CC1CCOC1(c1cccc(F)c1)c1cccc(F)c1] | 448.30 | (((2,2-bis(3- fluorophenyl)tetrahydrofuran-3- yl)methyl)(methyl)amino)methyl glycylglycinate |
| 112 | 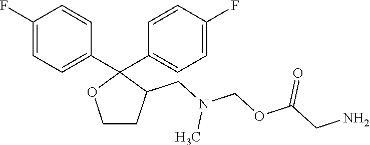 [SMILES: CN(COC(=O)CN)CC1CCOC1(c1ccc(F)cc1)c1ccc(F)cc1] | 391.26 | (((2,2-bis(4- fluorophenyl)tetrahydrofuran-3- yl)methyl)(methyl)amino)methyl glycinate |
| 113 | 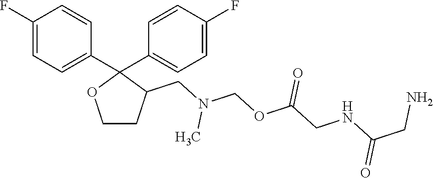 [SMILES: CN(COC(=O)CNC(=O)CN)CC1CCOC1(c1ccc(F)cc1)c1ccc(F)cc1] | 448.30 | (((2,2-bis(4- fluorophenyl)tetrahydrofuran-3- yl)methyl)(methyl)amino)methyl glycylglycinate |
| 114 | 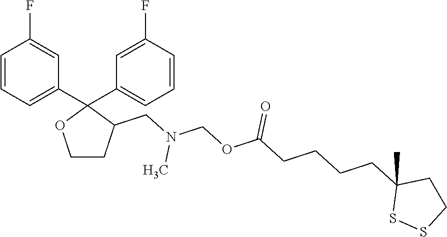 [SMILES: CN(COC(=O)CCCC[C@]1(C)CCSS1)CC1CCOC1(c1cccc(F)c1)c1cccc(F)c1] | 511.35 | 2-(((2,2-bis(4- fluorophenyl)tetrahydrofuran-3- yl)methyl)(methyl)amino)ethyl L-tyrosinate |
| 115 | 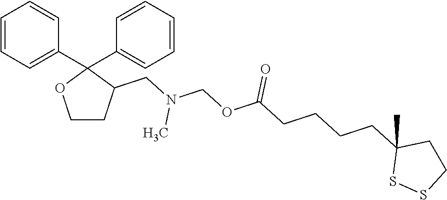 [SMILES: CN(COC(=O)CCCC[C@]1(C)CCSS1)CC1CCOC1(c1ccccc1)c1ccccc1] | 536.26 | (((2,2-bis(3- fluorophenyl)tetrahydrofuran-3- yl)methyl)(methyl)amino)methyl 5-((R)-3-methyl-1,2-dithiolan-3- yl)pentanoate |
| 116 | 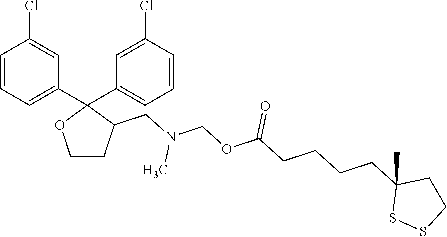 [SMILES: CN(COC(=O)CCCC[C@]1(C)CCSS1)CC1CCOC1(c1cccc(Cl)c1)c1cccc(Cl)c1] | 500.32 | (((2,2-diphenyltetrahydrofuran-3- yl)methyl)(methyl)amino)methyl 5-((R)-3-methyl-1,2-dithiolan-3- yl)pentanoate |
| 117 | 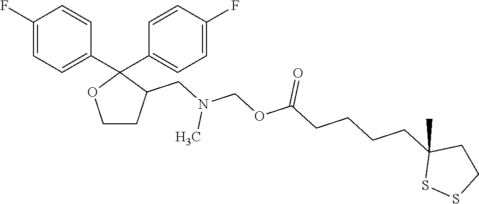 [SMILES: CN(COC(=O)CCCC[C@]1(C)CCSS1)CC1CCOC1(c1ccc(F)cc1)c1ccc(F)cc1] | 569.24 | (((2,2-bis(3- chlorophenyl)tetrahydrofuran-3- yl)methyl)(methyl)amino)methyl 5-((R)-3-methyl-1,2-dithiolan-3- yl)pentanoate |
| 118 | 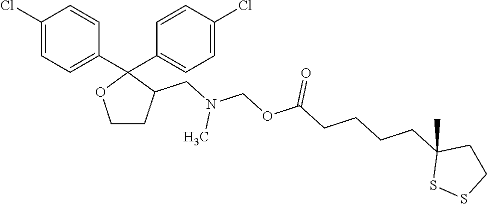 [SMILES: CN(COC(=O)CCCC[C@]1(C)CCSS1)CC1CCOC1(c1ccc(Cl)cc1)c1ccc(Cl)cc1] | 536.22 | (((2,2-bis(4- fluorophenyl)tetrahydrofuran-3- yl)methyl)(methyl)amino)methyl 5- ((R)-3-methyl-1,2-dithiolan-3- yl)pentanoate |
| 119 | 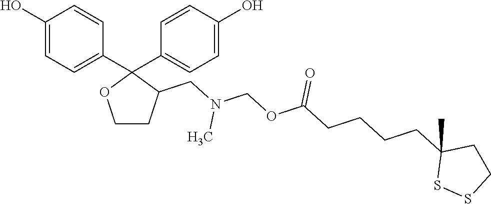 [SMILES: CN(COC(=O)CCCC[C@]1(C)CCSS1)CC1CCOC1(c1ccc(O)cc1)c1ccc(O)cc1] | 532.21 | (((2,2-bis(4- hydroxyphenyl)tetrahydrofuran-3- yl)methyl)(methyl)amino)methyl 5-((R)-3-methyl-1,2-dithiolan-3- yl)pentanoate |
| 120 | 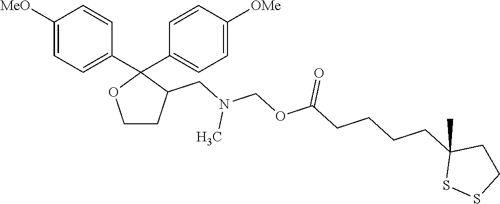 [SMILES: COc1ccc(C2(c3ccc(OC)cc3)OCCC2CN(C)COC(=O)CCCC[C@]2(C)CCSS2)cc1] | 560.25 | (((2,2-bis(4- methoxyphenyl)tetrahydrofuran-3- yl)methyl)(methyl)amino)methyl 5-((R)-3-methyl-1,2-dithiolan-3- yl)pentanoate |
| 121 | 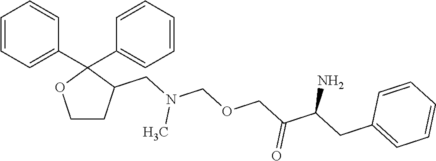 [SMILES: CN(COCC(=O)[C@@H](N)Cc1ccccc1)CC1CCOC1(c1ccccc1)c1ccccc1] | 459.36 | 2-(((2,2-diphenyltetrahydrofuran-3- yl)methyl)(methyl)amino)ethyl L- phenylalaninate |
| 122 | 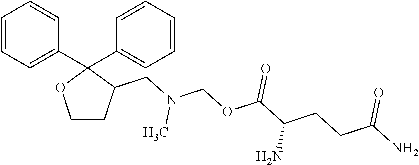 [SMILES: CN(COC(=O)[C@@H](N)CCC(N)=O)CC1CCOC1(c1ccccc1)c1ccccc1] | 426.33 | (((2,2-diphenyltetrahydrofuran-3- yl)methyl)(methyl)amino)methyl L-glutaminate |
| 123 | 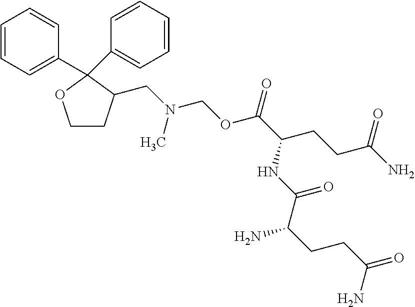 [SMILES: CN(COC(=O)[C@H](CCC(N)=O)NC(=O)[C@@H](N)CCC(N)=O)CC1CCOC1(c1ccccc1)c1ccccc1] | 554.39 | (((2,2-diphenyltetrahydrofuran-3- yl)methyl)(methyl)amino)methyl L-glutaminyl-L-glutaminate |
| 124 | 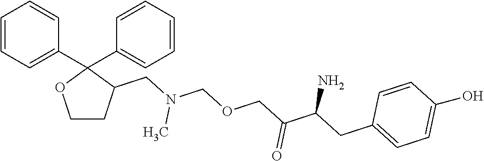 [SMILES: CN(COCC(=O)[C@@H](N)Cc1ccc(O)cc1)CC1CCOC1(c1ccccc1)c1ccccc1] | 475.35 | 2-(((2,2-diphenyltetrahydrofuran-3- yl)methyl)(methyl)amino)ethyl L-tyrosinate |
| 125 | 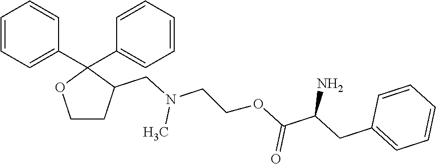 [SMILES: CN(CCOC(=O)[C@@H](N)Cc1ccccc1)CC1CCOC1(c1ccccc1)c1ccccc1] | 459.36 | 2-(((2,2-diphenyltetrahydrofuran-3- yl)methyl)(methyl)amino)ethyl L-phenylalaninate |
| 126 | 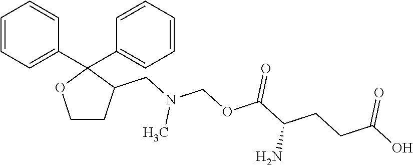 [SMILES: CN(COC(=O)[C@@H](N)CCC(=O)O)CC1CCOC1(c1ccccc1)c1ccccc1] | 427.32 | (4S)-4-amino-5-((((2,2- diphenyltetrahydrofuran-3- yl)methyl)(methyl)amino)methoxy)- 5-oxopentanoic acid |
| 127 | 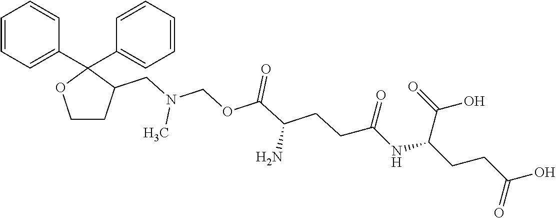 [SMILES: CN(COC(=O)[C@@H](N)CCC(=O)N[C@@H](CCC(=O)O)C(=O)O)CC1CCOC1(c1ccccc1)c1ccccc1] | 556.36 | ((4S)-4-amino-5-((((2,2- diphenyltetrahydrofuran-3- yl)methyl)(methyl)amino)methoxy)- 5-oxopentanoyl)-L-glutamic acid |
| 128 | 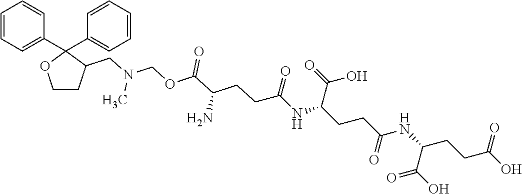 [SMILES: CN(COC(=O)[C@@H](N)CCC(=O)N[C@@H](CCC(=O)N[C@H](CCC(=O)O)C(=O)O)C(=O)O)CC1CCOC1(c1ccccc1)c1ccccc1] | 685.40 | (6S,11S,16R)-6-amino-1-(2,2- diphenyltetrahydrofuran-3-yl)-2- methyl-5,9,14-trioxo-4-oxa-2,10,15- triazaoctadecane-11,16,18- tricarboxylic acid |
| 129 | 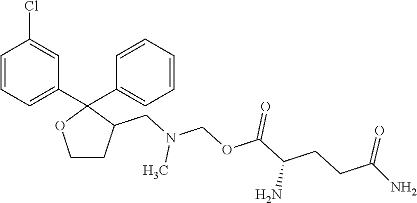 [SMILES: CN(COC(=O)[C@@H](N)CCC(N)=O)CC1CCOC1(c1ccccc1)c1cccc(Cl)c1] | 461.20 | (((2-(3-chlorophenyl)-2- phenyltetrahydrofuran-3- yl)methyl)(methyl)amino)methyl L-glutaminate |
| 130 | 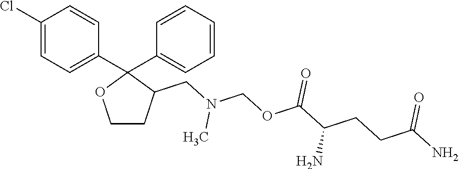 [SMILES: CN(COC(=O)[C@@H](N)CCC(N)=O)CC1CCOC1(c1ccccc1)c1ccc(Cl)cc1] | 461.19 | (((2-(4-chlorophenyl)-2- phenyltetrahydrofuran-3- yl)methyl)(methyl)amino)methyl L-glutaminate |
| 131 | 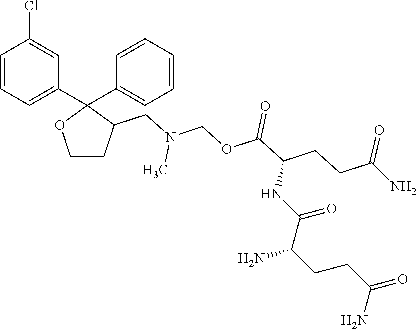 [SMILES: CN(COC(=O)[C@H](CCC(N)=O)NC(=O)[C@@H](N)CCC(N)=O)CC1CCOC1(c1ccccc1)c1cccc(Cl)c1] | 589.35 | (6S,11S,16R)-6-amino-1-(2,2- diphenyltetrahydrofuran-3-yl)-2- methyl-5,9,14-trioxo-4-oxa-2,10,15- triazaoctadecane-11,16,18- tricarboxylic acid |
| 132 | 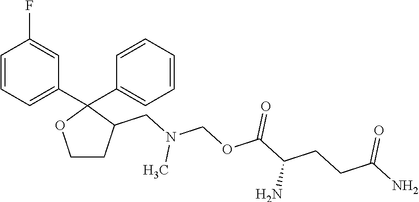 [SMILES: CN(COC(=O)[C@@H](N)CCC(N)=O)CC1CCOC1(c1ccccc1)c1cccc(F)c1] | 444.32 | (((2-(3-fluorophenyl)-2- phenyltetrahydrofuran-3- yl)methyl)(methyl)amino)methyl L-glutaminate |
| 133 | 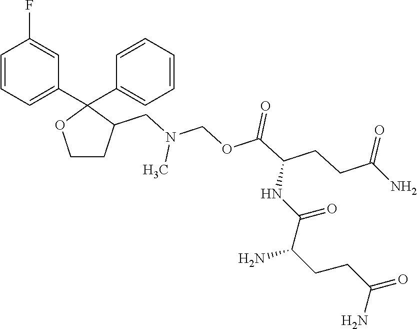 [SMILES: CN(COC(=O)[C@H](CCC(N)=O)NC(=O)[C@@H](N)CCC(N)=O)CC1CCOC1(c1ccccc1)c1cccc(F)c1] | 572.38 | (((2-(3-fluorophenyl)-2- phenyltetrahydrofuran-3- yl)methyl)(methyl)amino)methyl L-glutaminyl-L-glutainate |
| 134 | 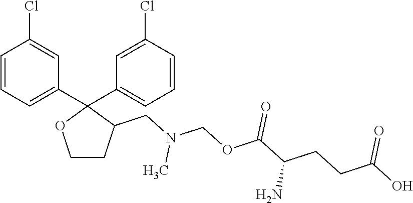 [SMILES: CN(COC(=O)[C@@H](N)CCC(=O)O)CC1CCOC1(c1cccc(Cl)c1)c1cccc(Cl)c1] | 496.24 | (4S)-4-amino-5-((((2,2-bis(3- chlorophenyl)tetrahydrofuran-3- yl)methyl)(methyl)amino)methoxy)- 5-oxopentanoic acid |
| 135 | 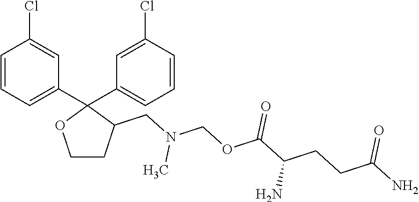 [SMILES: CN(COC(=O)[C@@H](N)CCC(N)=O)CC1CCOC1(c1cccc(Cl)c1)c1cccc(Cl)c1] | 495.25 | (((2,2-bis(3- chlorophenyl)tetrahydrofuran-3- yl)methyl)(methyl)amino)methyl L-glutaminate |
| 136 | 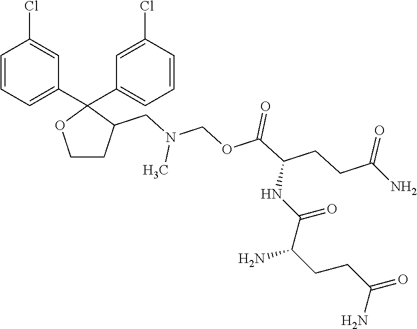 [SMILES: CN(COC(=O)[C@H](CCC(N)=O)NC(=O)[C@@H](N)CCC(N)=O)CC1CCOC1(c1cccc(Cl)c1)c1cccc(Cl)c1] | 623.31 | (((2,2-bis(3- chlorophenyl)tetrahydrofuran-3- yl)methyl)(methyl)amino)methyl L-glutaminyl-L-glutaminate |
| 137 | 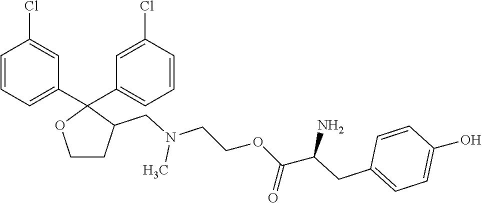 [SMILES: CN(CCOC(=O)[C@@H](N)Cc1ccc(O)cc1)CC1CCOC1(c1cccc(Cl)c1)c1cccc(Cl)c1] | 544.27 | 2-(((2,2-bis(3- chlorophenyl)tetrahydrofuran-3- yl)methyl)(methyl)amino)ethyl L-tyrosinate |
| 138 | 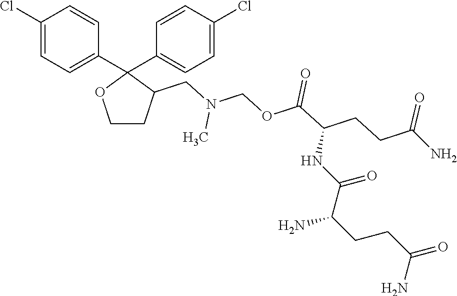 [SMILES: CN(COC(=O)[C@H](CCC(N)=O)NC(=O)[C@@H](N)CCC(N)=O)CC1CCOC1(c1ccc(Cl)cc1)c1ccc(Cl)cc1] | 623.31 | (6S,11S,16R)-6-amino-1-(2,2- diphenyltetrahydrofuran-3-yl)-2- methyl-5,9,14-trioxo-4-oxa-2,10,15- triazaoctadecane-11,16,18- tricarboxylic acid |
| 139 | 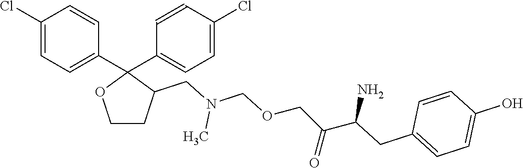 [SMILES: CN(COCC(=O)[C@@H](N)Cc1ccc(O)cc1)CC1CCOC1(c1ccc(Cl)cc1)c1ccc(Cl)cc1] | 544.27 | 2-(((2,2-bis(4- chlorophenyl)tetrahydrofuran-3- yl)methyl)(methyl)amino)ethyl L-tyrosinate |
| 140 | 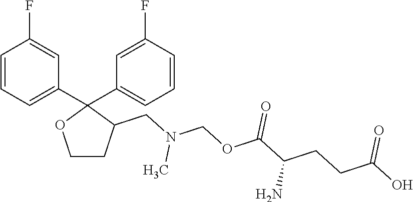 [SMILES: CN(COC(=O)[C@@H](N)CCC(=O)O)CC1CCOC1(c1cccc(F)c1)c1cccc(F)c1] | 463.23 | (4S)-4-amino-5-((((2,2-bis(3- fluorophenyl)tetrahydrofuran-3- yl)methyl)(methyl)amino)methoxy)- 5-oxopentanoic acid |
| 141 | 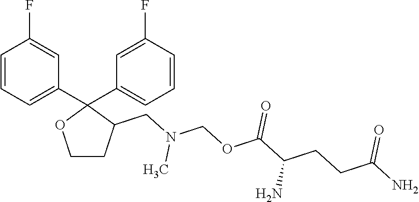 [SMILES: CN(COC(=O)[C@@H](N)CCC(N)=O)CC1CCOC1(c1cccc(F)c1)c1cccc(F)c1] | 462.34 | (((2,2-bis(3- fluorophenyl)tetrahydrofuran-3- yl)methyl)(methyl)amino)methyl L-glutaminate |
| 142 | 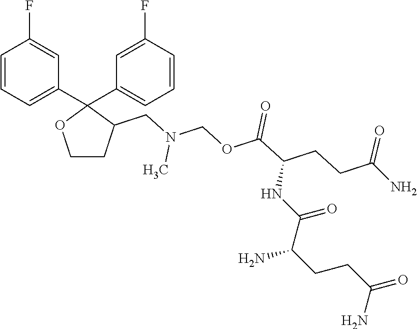 [SMILES: CN(COC(=O)[C@H](CCC(N)=O)NC(=O)[C@@H](N)CCC(N)=O)CC1CCOC1(c1cccc(F)c1)c1cccc(F)c1] | 590.37 | (((2,2-bis(3- fluorophenyl)tetrahydrofuran-3- yl)methyl)(methyl)amino)methyl L-glutaminyl-L-glutaminate |
| 143 | 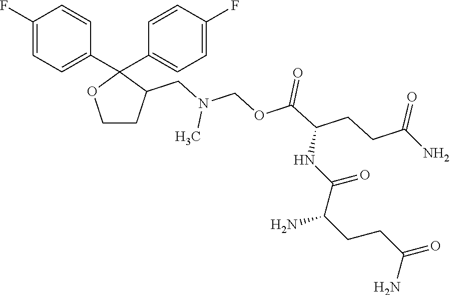 [SMILES: CN(COC(=O)[C@H](CCC(N)=O)NC(=O)[C@@H](N)CCC(N)=O)CC1CCOC1(c1ccc(F)cc1)c1ccc(F)cc1] | 599.37 | (((2,2-bis(4- fluorophenyl)tetrahydrofuran-3- yl)methyl)(methyl)amino)methyl L-glutaminyl-L-glutaminate |
| 144 | 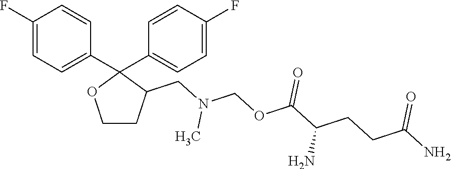 [SMILES: CN(COC(=O)[C@@H](N)CCC(N)=O)CC1CCOC1(c1ccc(F)cc1)c1ccc(F)cc1] | 462.31 | (((2,2-bis(4- fluorophenyl)tetrahydrofuran-3- yl)methyl)(methyl)amino)methyl L-glutaminate |
| 145 | 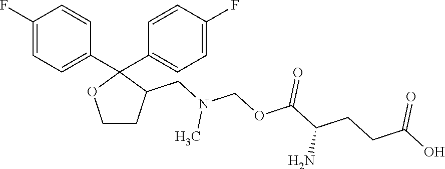 [SMILES: CN(COC(=O)[C@@H](N)CCC(=O)O)CC1CCOC1(c1ccc(F)cc1)c1ccc(F)cc1] | 463.30 | (4S)-4-amino-5-((((2,2-bis(4- fluorophenyl)tetrahydrofuran-3- yl)methyl)(methyl)amino)methoxy)- 5-oxopentanoic acid |
| 146 | 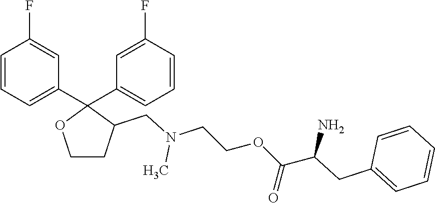 [SMILES: CN(CCOC(=O)[C@@H](N)Cc1ccccc1)CC1CCOC1(c1cccc(F)c1)c1cccc(F)c1] | 495.34 | 2-(((2,2-bis(3- fluorophenyl)tetrahydrofuran-3- yl)methyl)(methyl)amino)ethyl L-phenylalaninate |
| 147 | 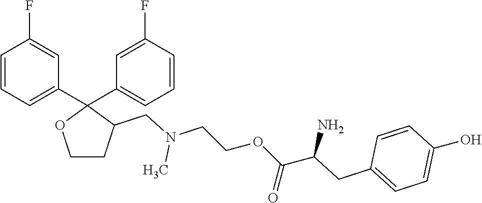 [SMILES: CN(CCOC(=O)[C@@H](N)Cc1ccc(O)cc1)CC1CCOC1(c1cccc(F)c1)c1cccc(F)c1] | 511.33 | 2-(((2,2-bis(3- fluorophenyl)tetrahydrofuran-3- yl)methyl)(methyl)amino)ethyl L-tyrosinate |
| 148 | 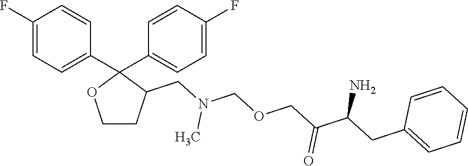 [SMILES: CN(COCC(=O)[C@@H](N)Cc1ccccc1)CC1CCOC1(c1ccc(F)cc1)c1ccc(F)cc1] | 495.36 | 2-(((2,2-bis(4- fluorophenyl)tetrahydrofuran-3- yl)methyl)(methyl)amino)ethyl L-phenylalaninate |
| 149 | 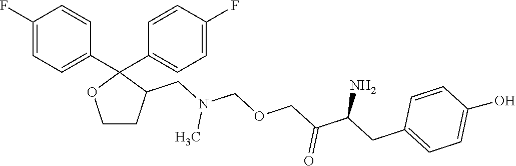 [SMILES: CN(COCC(=O)[C@@H](N)Cc1ccc(O)cc1)CC1CCOC1(c1ccc(F)cc1)c1ccc(F)cc1] | 511.35 | 2-(((2,2-bis(4- fluorophenyl)tetrahydrofuran-3- yl)methyl)(methyl)amino)ethyl L-tyrosinate |
| 150 | 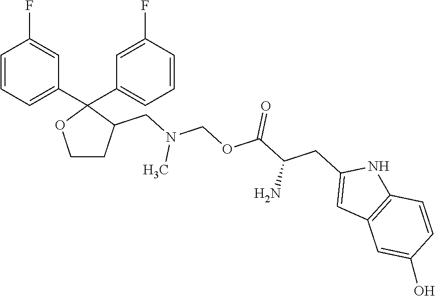 [SMILES: CN(COC(=O)[C@@H](N)Cc1cc2cc(O)ccc2[nH]1)CC1CCOC1(c1cccc(F)c1)c1cccc(F)c1] | 536.23 | (((2,2-bis(3- fluorophenyl)tetrahydrofuran-3- yl)methyl)(methyl)amino)methyl (2S)-2-amino-3-(5-hydroxy-1H- indol-2-yl)propanoate |
| 151 | 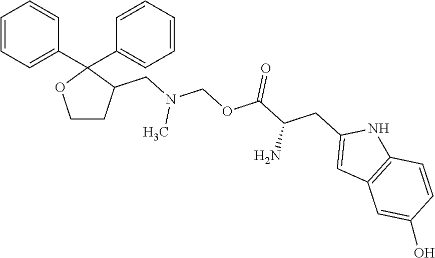 [SMILES: CN(COC(=O)[C@@H](N)Cc1cc2cc(O)ccc2[nH]1)CC1CCOC1(c1ccccc1)c1ccccc1] | 500.25 | (((2,2-diphenyltetrahydrofuran-3- yl)methyl)(methyl)amino)methyl (2S)-2-amino-3-(5-hydroxy-1H- indol-2-yl)propanoate |
| 152 | 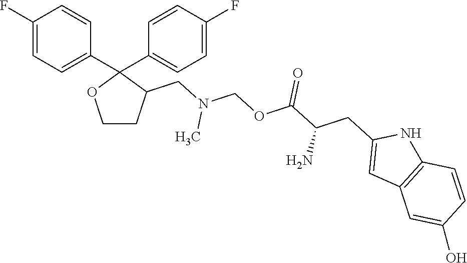 [SMILES: CN(COC(=O)[C@@H](N)Cc1cc2cc(O)ccc2[nH]1)CC1CCOC1(c1ccc(F)cc1)c1ccc(F)cc1] | 536.23 | (((2,2-bis(4- fluorophenyl)tetrahydrofuran-3- yl)methyl)(methyl)amino)methyl (2S)-2-amino-3-(5-hydroxy-1H- indol-2-yl)propanoate |
| 153 | 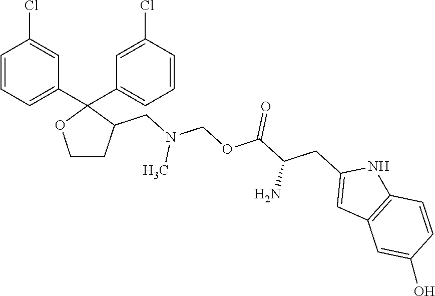 [SMILES: CN(COC(=O)[C@@H](N)Cc1cc2cc(O)ccc2[nH]1)CC1CCOC1(c1cccc(Cl)c1)c1cccc(Cl)c1] | 569.17 | (((2,2-bis(3- chlorophenyl)tetrahydrofuran-3- yl)methyl)(methyl)amino)methyl (2S)-2-amino-3-(5-hydroxy-1H- indol-2-yl)propanoate |
| 154 | 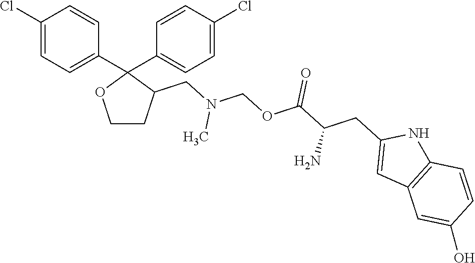 [SMILES: CN(COC(=O)[C@@H](N)Cc1cc2cc(O)ccc2[nH]1)CC1CCOC1(c1ccc(Cl)cc1)c1ccc(Cl)cc1] | 569.17 | (((2,2-bis(4- chlorophenyl)tetrahydrofuran-3- yl)methyl)(methyl)amino)methyl (2S)-2-amino-3-(5-hydroxy-1H- indol-2-yl)propanoate |
| 155 | 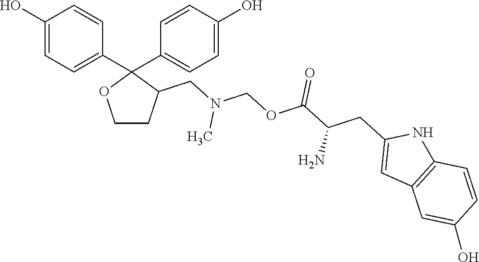 [SMILES: CN(COC(=O)[C@@H](N)Cc1cc2cc(O)ccc2[nH]1)CC1CCOC1(c1ccc(O)cc1)c1ccc(O)cc1] | 532.24 | (((2,2-bis(4- hydroxyphenyl)tetrahydrofuran-3- yl)methyl)(methyl)amino)methyl (2S)-2-amino-3-(5-hydroxy-1H- indol-2-yl)propanoate |
| 156 | 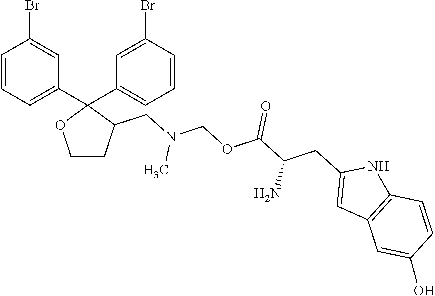 [SMILES: CN(COC(=O)[C@@H](N)Cc1cc2cc(O)ccc2[nH]1)CC1CCOC1(c1cccc(Br)c1)c1cccc(Br)c1] | 659.04 | (((2,2-bis(3- bromophenyl)tetrahydrofuran-3- yl)methyl)(methyl)amino)methyl (2S)-2-amino-3-(5-hydroxy-1H- indol-2-yl)propanoate |
| 157 | 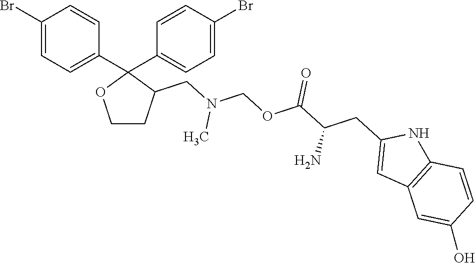 [SMILES: CN(COC(=O)[C@@H](N)Cc1cc2cc(O)ccc2[nH]1)CC1CCOC1(c1ccc(Br)cc1)c1ccc(Br)cc1] | 659.06 | (((2,2-bis(4- bromophenyl)tetrahydrofuran-3- yl)methyl)(methyl)amino)methyl (2S)-2-amino-3-(5-hydroxy-1H- indol-2-yl)propanoate |
| 158 | 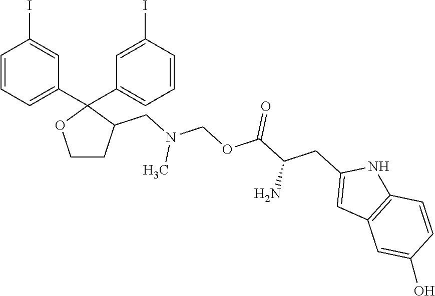 [SMILES: CN(COC(=O)[C@@H](N)Cc1cc2cc(O)ccc2[nH]1)CC1CCOC1(c1cccc(I)c1)c1cccc(I)c1] | 752.04 | (((2,2-bis(3- iodophenyl)tetrahydrofuran-3- yl)methyl)(methyl)amino)methyl (2S)-2-amino-3-(5-hydroxy-1H- indol-2-yl)propanoate |
| 159 | 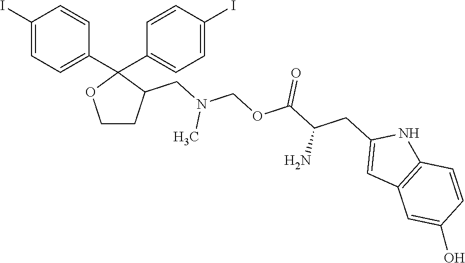 [SMILES: CN(COC(=O)[C@@H](N)Cc1cc2cc(O)ccc2[nH]1)CC1CCOC1(c1ccc(I)cc1)c1ccc(I)cc1] | 753.05 | (((2,2-bis(4- iodophenyl)tetrahydrofuran-3- yl)methyl)(methyl)amino)methyl (2S)-2-amino-3-(5-hydroxy-1H- indol-2-yl)propanoate |
| 160 | 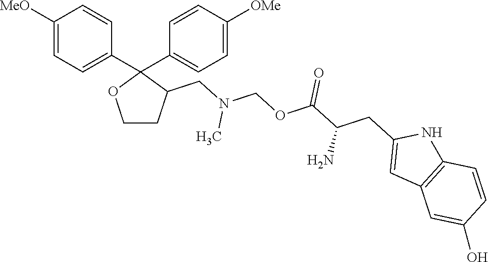 [SMILES: COc1ccc(C2(c3ccc(OC)cc3)OCCC2CN(C)COC(=O)[C@@H](N)Cc2cc3cc(O)ccc3[nH]2)cc1] | 560.27 | (((2,2-bis(4- methoxyphenyl)tetrahydrofuran-3- yl)methyl)(methyl)amino)methyl (2S)-2-amino-3-(5-hydroxy-1H- indol-2-yl)propanoate |
| 161 | 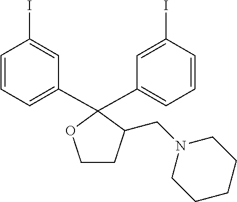 [SMILES: Ic1cccc(C2(c3cccc(I)c3)OCCC2CN2CCCCC2)c1] | 574.01 | 1-((2,2-bis(3- iodophenyl)tetrahydrofuran-3- yl)methyl)piperidine |
| 162 | 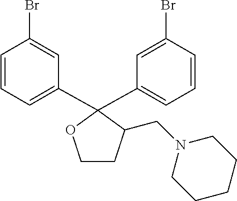 [SMILES: Brc1cccc(C2(c3cccc(Br)c3)OCCC2CN2CCCCC2)c1] | 481.03 | 1-((2,2-bis(3- bromophenyl)tetrahydrofuran-3- yl)methyl)piperidine |
| 163 | 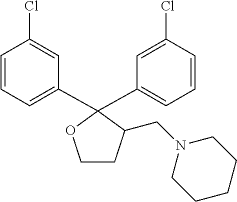 [SMILES: Clc1cccc(C2(c3cccc(Cl)c3)OCCC2CN2CCCCC2)c1] | 391.13 | 1-((2,2-bis(3- chlorophenyl)tetrahydrofuran-3- yl)methyl)piperidine |
| 164 | 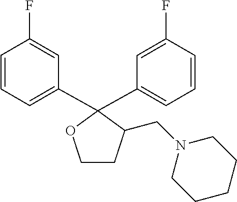 [SMILES: Fc1cccc(C2(c3cccc(F)c3)OCCC2CN2CCCCC2)c1] | 358.19 | 1-((2,2-bis(3- fluorophenyl)tetrahydrofuran-3- yl)methyl)piperidine |
| 165 | 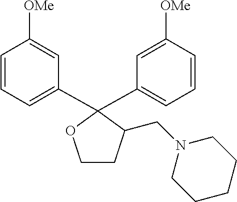 [SMILES: COc1cccc(C2(c3cccc(OC)c3)OCCC2CN2CCCCC2)c1] | 382.23 | 1-((2,2-bis(3- methoxyphenyl)tetrahydrofuran-3- yl)methyl)piperidine |
| 166 | 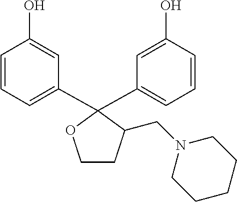 [SMILES: Oc1cccc(C2(c3cccc(O)c3)OCCC2CN2CCCCC2)c1] | 354.20 | 3,3′-(3-(piperidin-1- ylmethyl)tetrahydrofuran-2,2- diyl)diphenol |
| 167 | 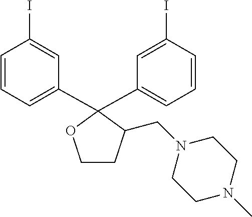 [SMILES: CN1CCN(CC2CCOC2(c2cccc(I)c2)c2cccc(I)c2)CC1] | 589.02 | 1-((2,2-bis(3- iodophenyl)tetrahydrofuran-3- yl)methyl)-4-methylpiperazine |
| 168 | 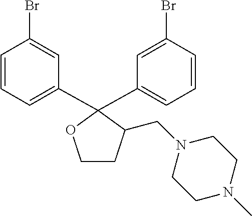 [SMILES: CN1CCN(CC2CCOC2(c2cccc(Br)c2)c2cccc(Br)c2)CC1] | 496.04 | 1-((2,2-bis(3- bromophenyl)tetrahydrofuran-3- yl)methyl)-4-methylpiperazine |
| 169 | 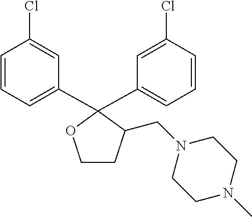 [SMILES: CN1CCN(CC2CCOC2(c2cccc(Cl)c2)c2cccc(Cl)c2)CC1] | 406.14 | 1-((2,2-bis(3- chlorophenyl)tetrahydrofuran-3- yl)methyl)-4-methylpiperazine |
| 170 | 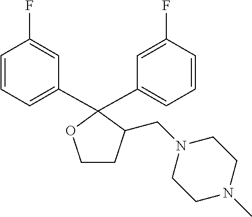 [SMILES: CN1CCN(CC2CCOC2(c2cccc(F)c2)c2cccc(F)c2)CC1] | 373.20 | 1-((2,2-bis(3- fluorophenyl)tetrahydrofuran-3- yl)methyl)-4-methylpiperazine |
| 171 | 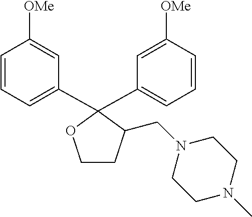 [SMILES: COc1cccc(C2(c3cccc(OC)c3)OCCC2CN2CCN(C)CC2)c1] | 397.24 | 1-((2,2-bis(3- methoxyphenyl)tetrahydrofuran-3- yl)methyl)-4-methylpiperazine |
| 172 | 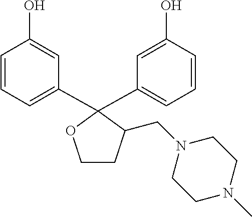 [SMILES: CN1CCN(CC2CCOC2(c2cccc(O)c2)c2cccc(O)c2)CC1] | 369.21 | 3,3′-(3-((4-methylpiperazin-1- yl)methyl)tetrahydrofuran-2,2- diyl)diphenol |

### Example 173

The compounds of Examples 1-172 are tested. N,N-dimethyltrytamine (DMT) is used as control. The compounds of the Examples 1-172 show good efficiency or have improved PK/PD over DMT. These compounds can be used to treatment of major depressive disorder (MDD), anxiety, depression, general anxiety disorder, social phobia, panic disorder, vasomotor symptoms, diabetic neuropathy, epilepsy, bipolar disorder, migraine, schizophrenia, cancer, menopause, HIV, neurological diseases and familial adenomatous polyposis.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for cancer or others. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

### Radioligand Binding Assay:

The binding affinity of the compounds to Sig-1R was studied using transfected HEK293 cell membranes and tritium labeled [3H](+)-pentazocine (PerkinElmer) as the competitive radioligand, following the protocol described by Navarro et al (Int. J. Mol. Sci. 2022, 23, 6737). Briefly, the assay was carried out using membrane suspension and Tritium-labeled (+)-pentazocine (2 nM) in 96-well membrane filtration plate. Haloperidol (10 uM) was used to determine the total and non-specific binding respectively. Testing compound was incubated at different concentrations with the membrane suspension in a TrisHCl buffer (50 mM, pH 7.4, 100 ul) at 37° C. for 2 hours. The plate was filtered and washed with ice-cold buffer. The bound radioactivity on the membrane filter was measured in a MicroBeta scintillation counter. The affinity of compounds was ranked based on the dose response of their binding curves.

1. A compound having the following formula (I), an isomer thereof, or a pharmaceutical acceptable solvate thereof, 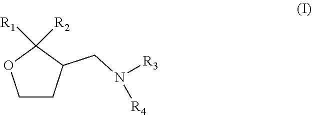
wherein
R₁ is 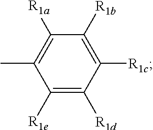
R₂ is 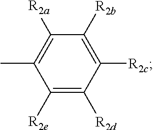
R₁ₐ, R_{1b}, R_{1c}, R_{1d}, R₁ₑ, R₂ₐ, R_{2b}, R_{2c}, R_{2d}, and R₂ₑ are each independently selected from the group consisting of —H, —F, —Cl, —Br, —I, —OH, —OCH₃, —CH₃, —CF₃, —CHF₂, —CH₂F, —CD₃, —CN, and 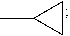
R₃ and R₄ are independently selected from the group consisting of —CH₃, —CD₃, —CF₃, —CHF₂, —CH₂F, —(CH₂)ₙ—O—R₅, and —CO—R₆, n is 1 or 2, provided that when R₁ₐ, R_{1b}, R_{1c}, R_{1d}, R₁ₑ, R₂ₐ, R_{2b}, R_{2c}, R_{2d}, and R₂ₑ are all —H, at least one of R₃ and R₄ is —CD₃, —CF₃, —CHF₂, —CH₂F, —(CH₂)ₙ—O—R₅, or —CO—R₆; or R₃ and R₄ form a 5-membered or 6-membered heterocyclyl group; and
R₅ is 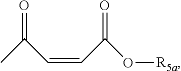
—CO—CH(R_{5b})—(CH₂)ₘ—R_{5c}, or —CH₂—R_{5d}; R₅ₐ is —H or a C1-6 alkyl group; R_{5b} is —H, —NH₂ or 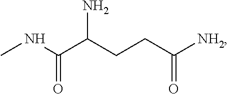
m is 0, 1, 2, or 3, R_{5c} is —COOH, —CONH₂, —NH₂, —NH—CO—CH₂—NH₂, 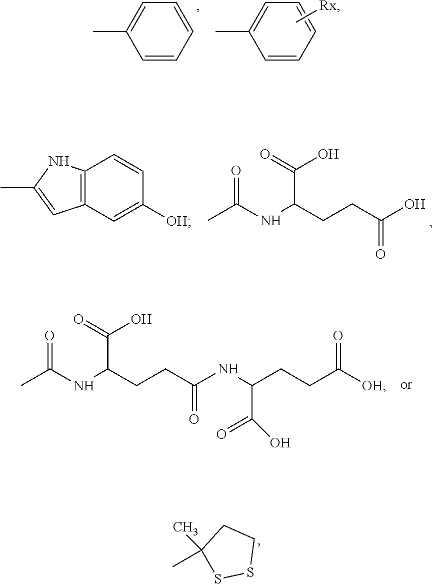
Rₓ is —OH, —NH₂, —F, —Cl, —Br, or —CN; R_{5d} is —CH₂CH₂NH₂, —CH₂CH₂CONH₂, 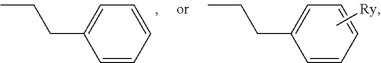
R_{y} is —OH, —NH₂, —F, —Cl, —Br, or —CN; and
R₆ is —CH₂CH₂NH₂, —CH₂CH₂CONH₂,

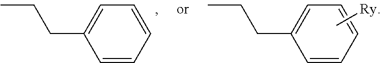

2. The compound of claim 1, wherein R₁ is 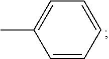
R₂ is 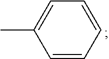
R₃ is selected from the group consisting of —CD₃, —CF₃, —CHF₂, and —CH₂F; and R₄ is selected from the group consisting of —CH₃, —CD₃, —CF₃, —CHF₂, and —CH₂F.

3. The compound of claim 2, wherein the compound is the selected from the group consisting of

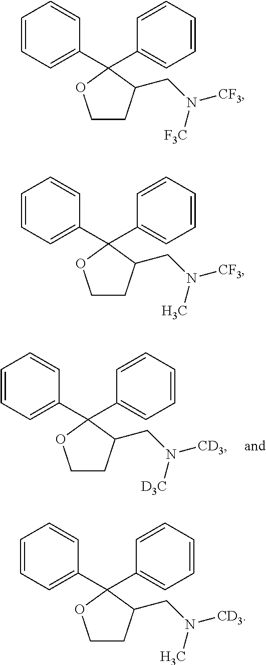

4. The compound of claim 1, wherein R₁ is 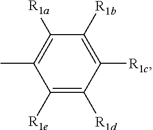
one of R₁ₐ, R_{1b}, R_{1c}, R_{1d}, and R₁ₑ is selected from the group consisting of —F, —Cl, —Br, —I, —OH, —OCH₃, —CH₃, —CF₃, —CHF₂, —CH₂F, —CD₃, —CN, and 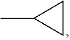
and remaining four of R₁ₐ, R_{1b}, R_{1c}, R_{1d}, and R₁ₑ are selected from the group consisting of —H, —F, —Cl, —Br, —I, —OH, —OCH₃, —CH₃, —CF₃, —CHF₂, —CH₂F, —CD₃, —CN; and/or
R₂ is 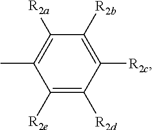
one of R₂ₐ, R_{2b}, R_{2c}, R_{2d}, and R₂ₑ is selected from the group consisting of —F, —Cl, —Br, —I, —OH, —OCH₃, —CH₃, —CF₃, —CHF₂, —CH₂F, —CD₃, —CN, and 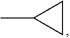
and remaining four of R₂ₐ, R_{2b}, R_{2c}, R_{2d}, and R₂ₑ are selected from the group consisting of —H, —F, —Cl, —Br, —I, —OH, —OCH₃, —CH₃, —CF₃, —CHF₂, —CH₂F, —CD₃, —CN.

5. The compound of claim 4, wherein the remaining four of R₁ₐ, R_{1b}, R_{1c}, R_{1d}, and R₁ₑ are —H; and/or the remaining four of R₂ₐ, R_{2b}, R_{2c}, R_{2d}, and R₂ₑ are —H.

6. The compound of claim 1, wherein R₃ and R₄ form a group selected from the group consisting of

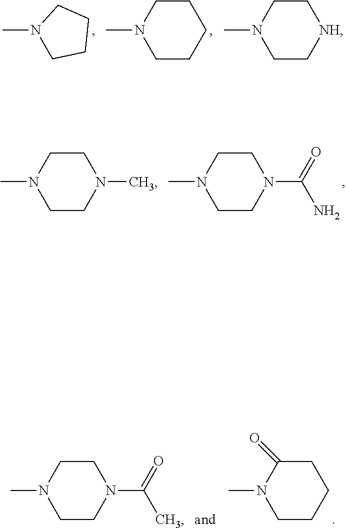

7. The compound of claim 1, wherein R₃ is —CH₃, —CD₃, —CF₃, —CHF₂, or —CH₂F; R₄ is —CH₂—O—R₅; R₅ is 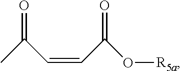
and R₅ₐ is —H, —CH₃, —CH₂CH₃, or —C(CH₃)₃.

8. The compound of claim 1, wherein R₃ is —CH₃, —CD₃, —CF₃, —CHF₂, or —CH₂F; R₄ is —(CH₂)ₙ—O—R₅; R₅ is —CO—CH(R_{5b})—(CH₂)ₘ—R_{5c}; n is 1 or 2; m is 1 or 2; R_{5b} is —NH₂ or 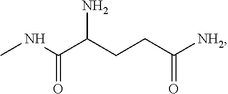
and R_{5c} is —COOH, —CONH₂, 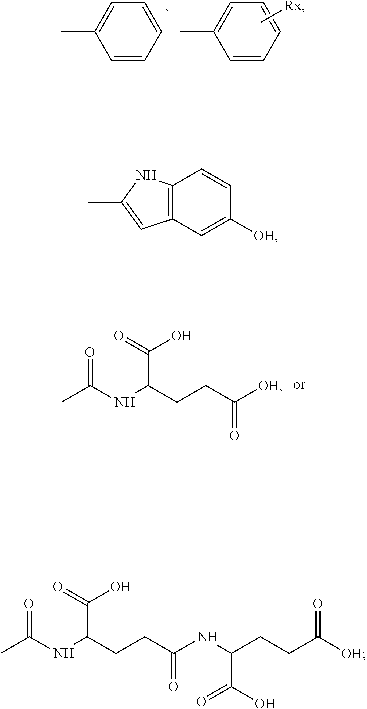
Rₓ is —OH, —NH₂, —F, —Cl, —Br, or —CN.

9. The compound of claim 1, wherein R₃ is —CH₃, —CD₃, —CF₃, —CHF₂, or —CH₂F; R₄ is —CH₂—O—R₅; R₅ is —CO—CH₂—(CH₂)ₘ—R_{5c}; m is 0, 1, 2, or 3; R_{5c} is —NH₂, —NH—CO—CH₂—NH₂, 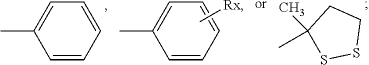
Rₓ is —OH, —NH₂, —F, —Cl, —Br, or —CN.

10. The compound of claim 1, wherein R₃ is —CH₃, —CD₃, —CF₃, —CHF₂, or —CH₂F; R₄ is —CH₂—O—R₅; R₅ is —CH₂—R_{5d}; R_{5d} is —CH₂CH₂NH₂, —CH₂CH₂CONH₂, 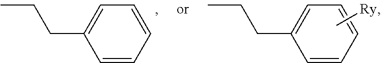
R_{y} is —OH, —NH₂, —F, —Cl, —Br, or —CN.

11. The compound of claim 1, wherein R₃ is —CH₃, —CD₃, —CF₃, —CHF₂, or —CH₂F; R₄ is and —CO—R₆; R₆ is —CH₂CH₂NH₂, 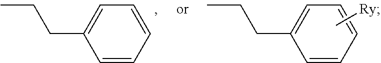
R_{y} is —OH, —NH₂, —F, —Cl, —Br, or —CN.

12. The compound of claim 1, wherein the compound is selected from the group consisting of

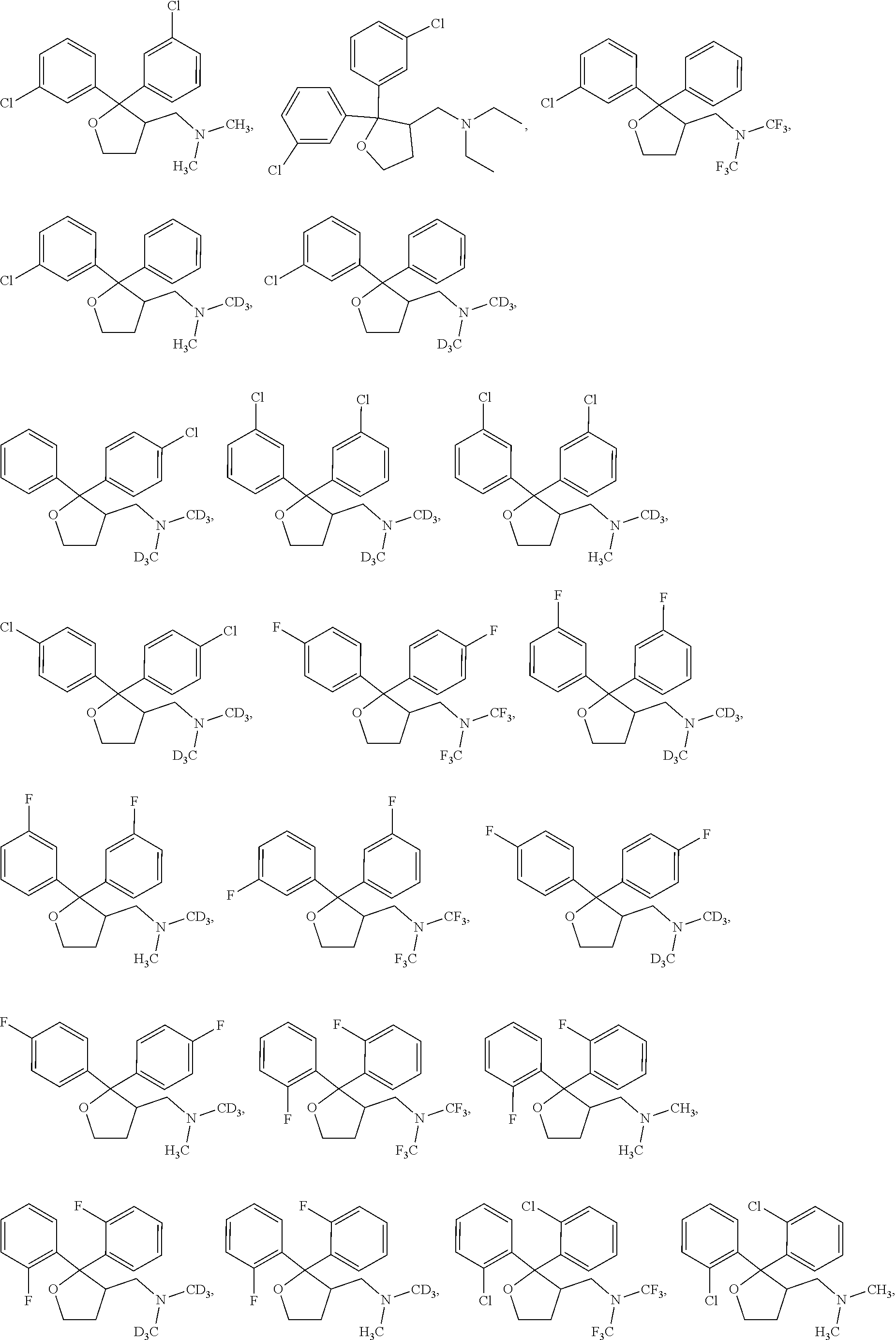

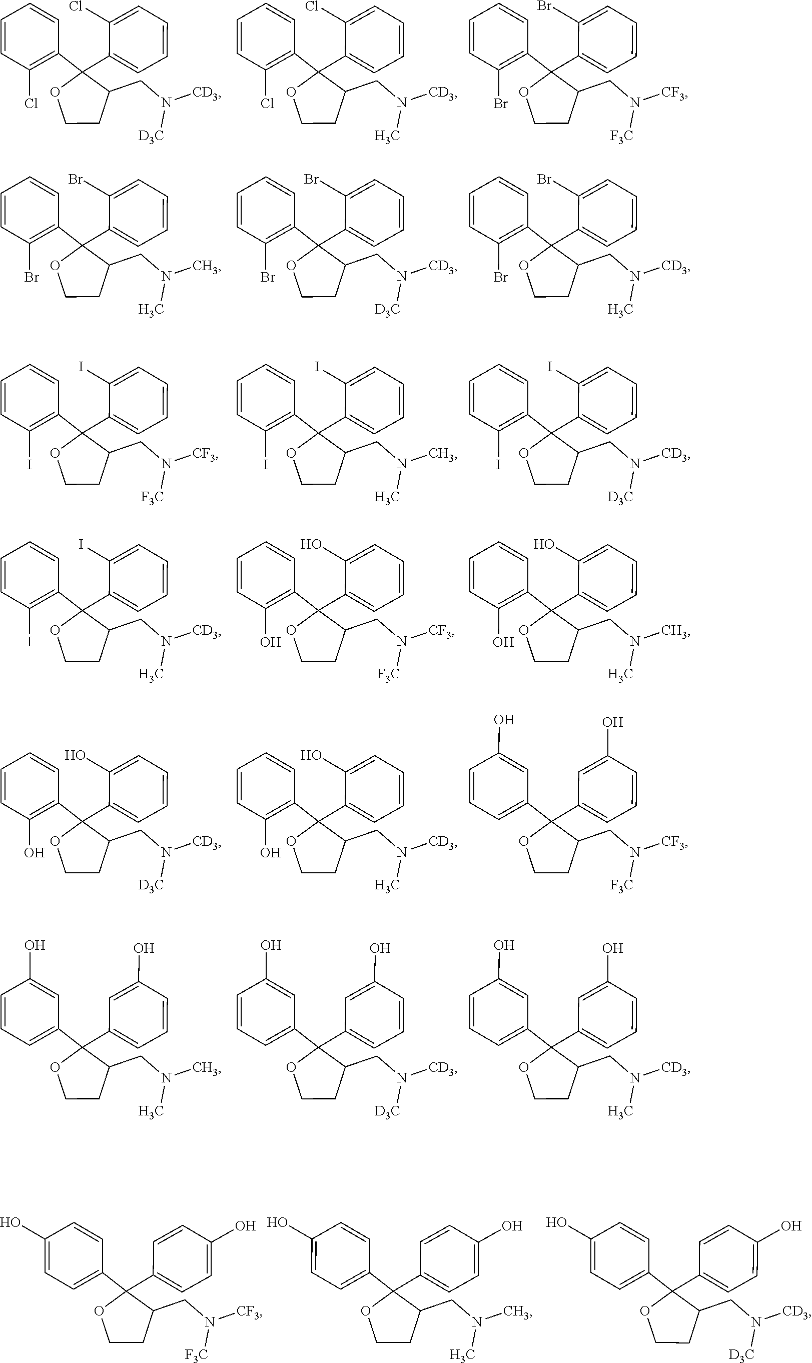

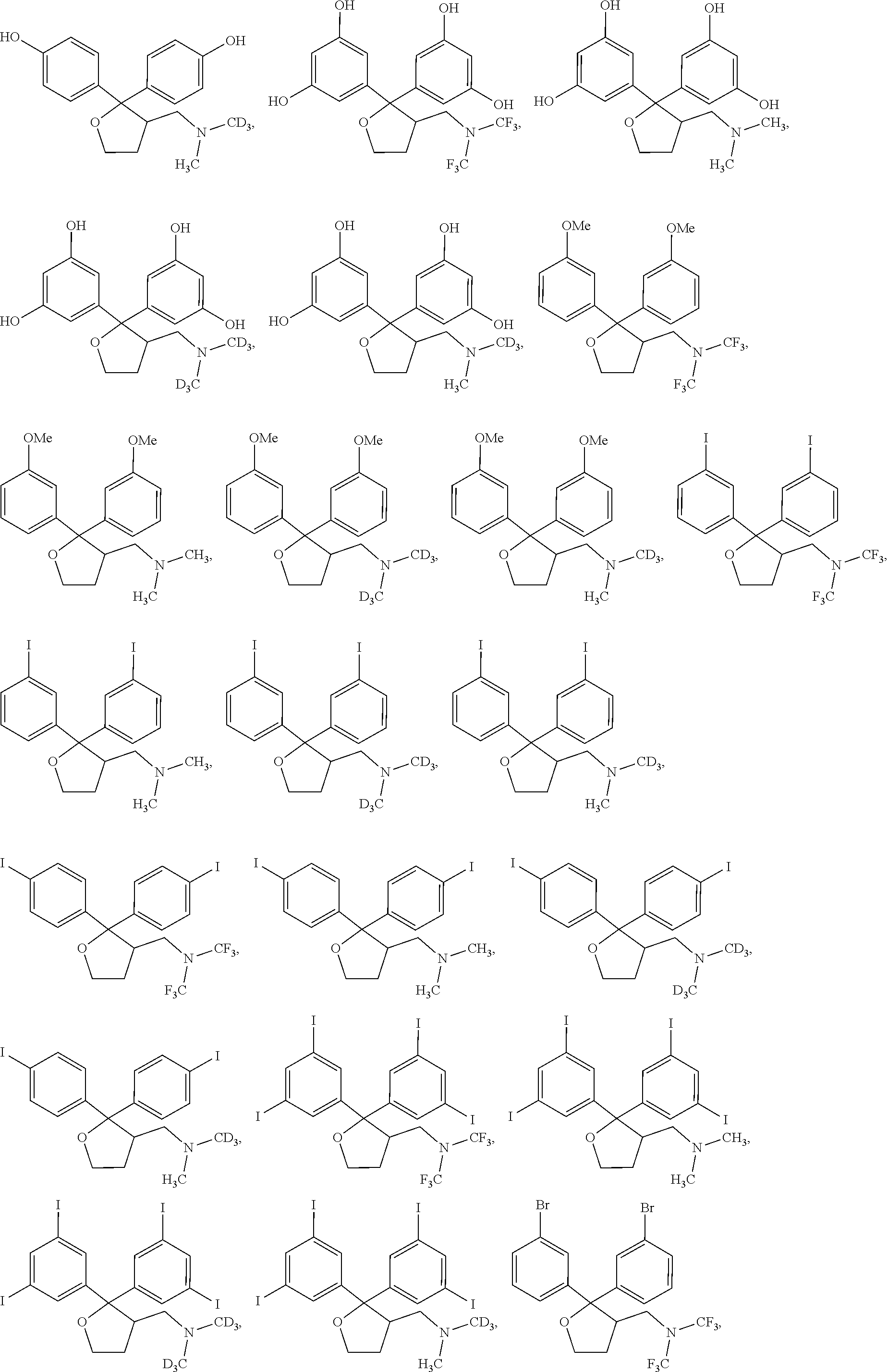

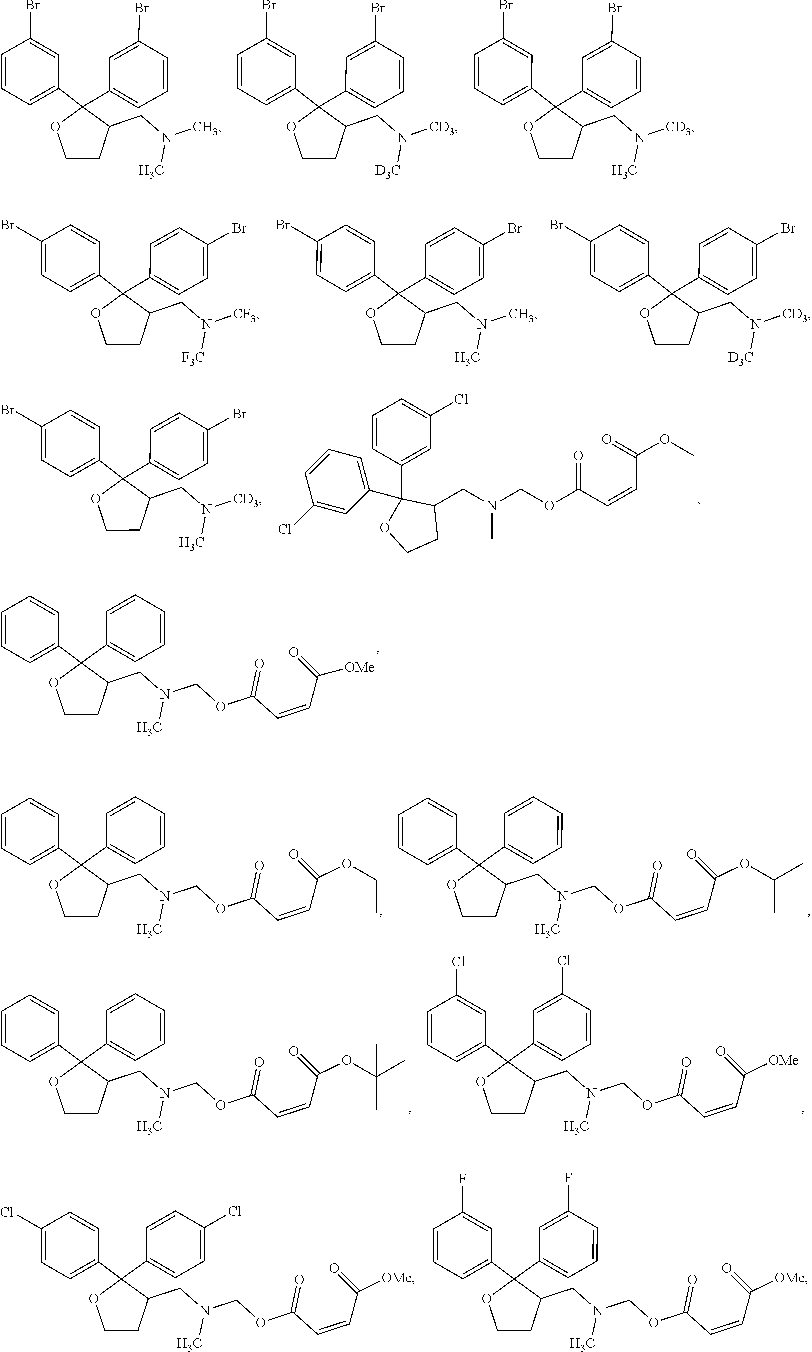

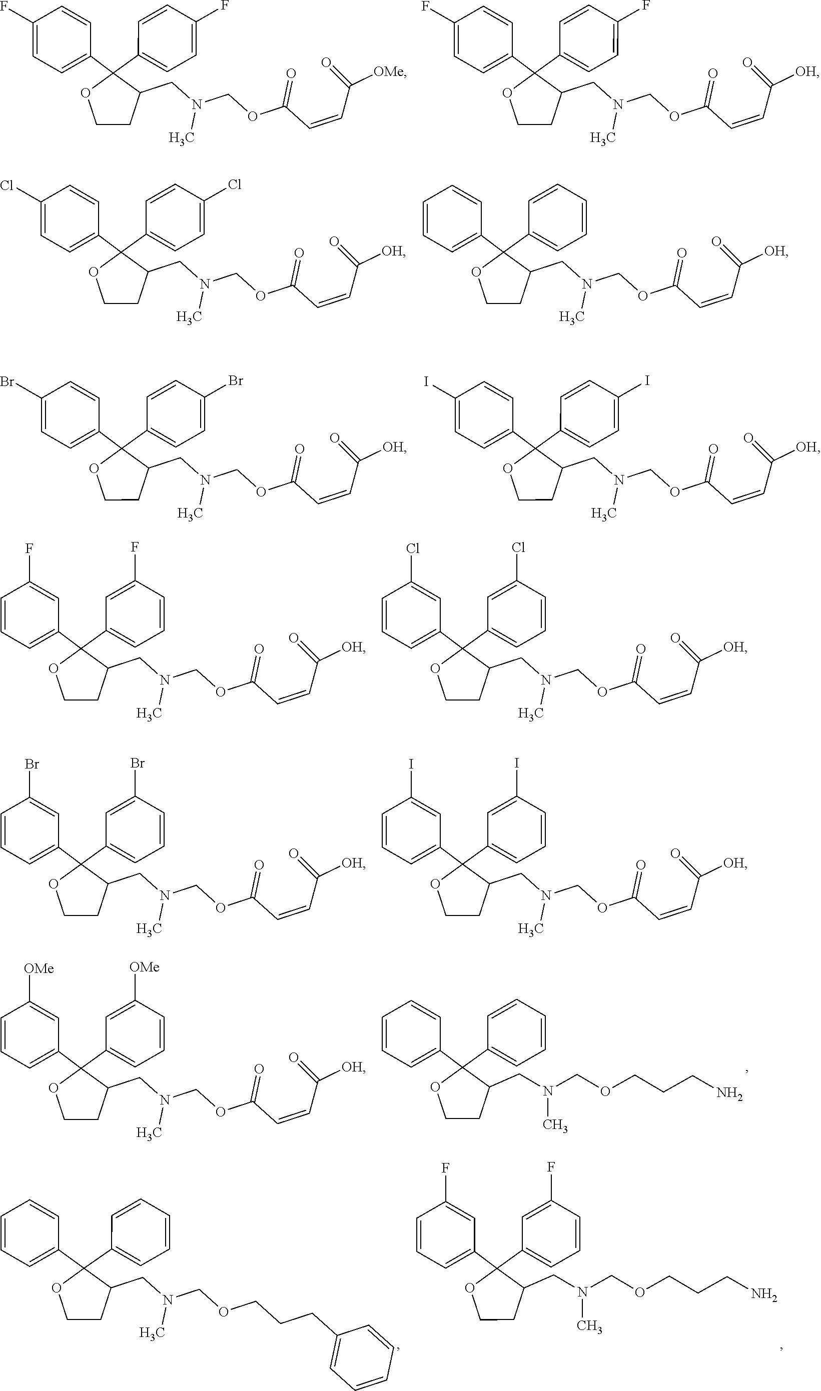

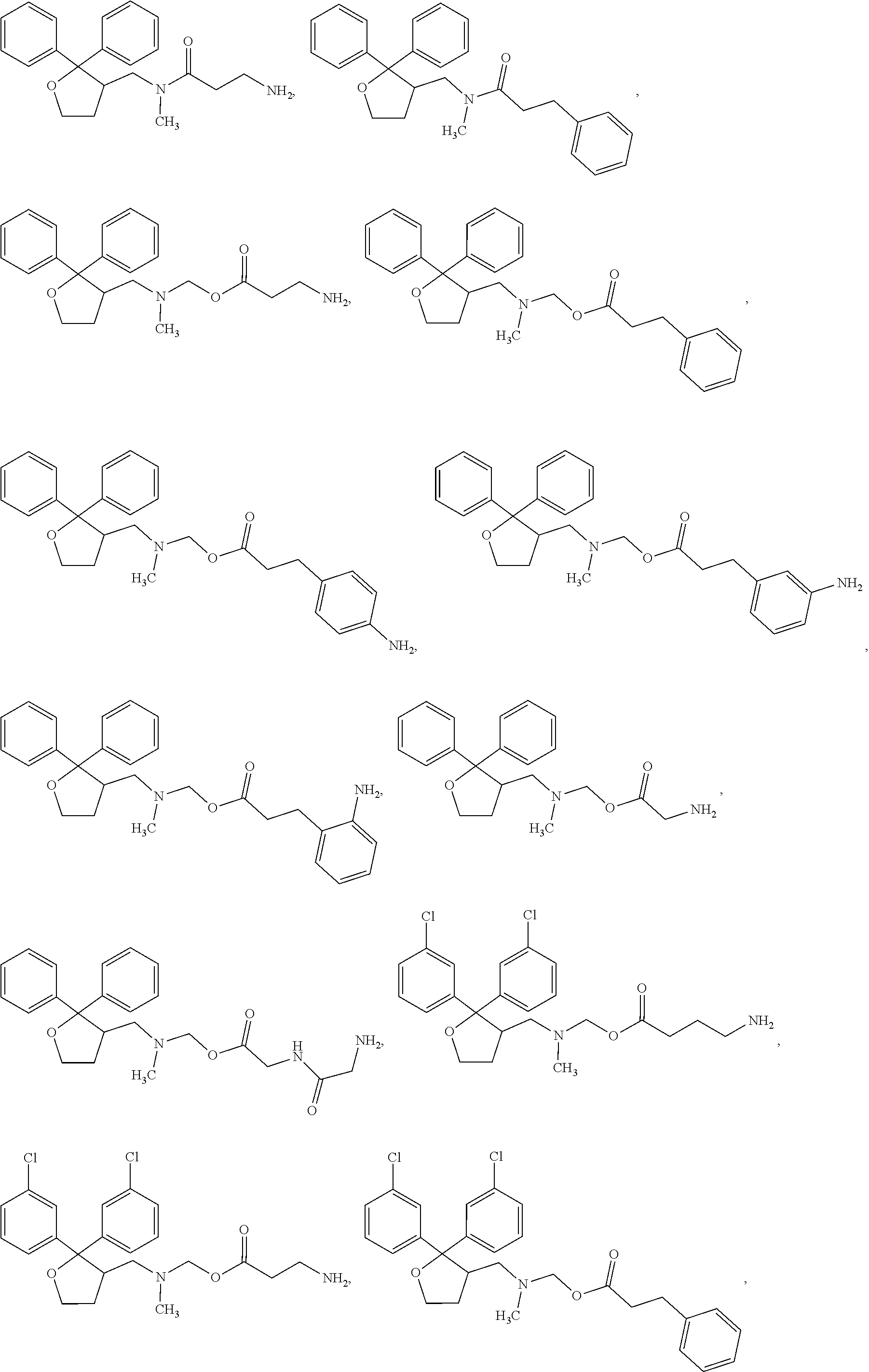

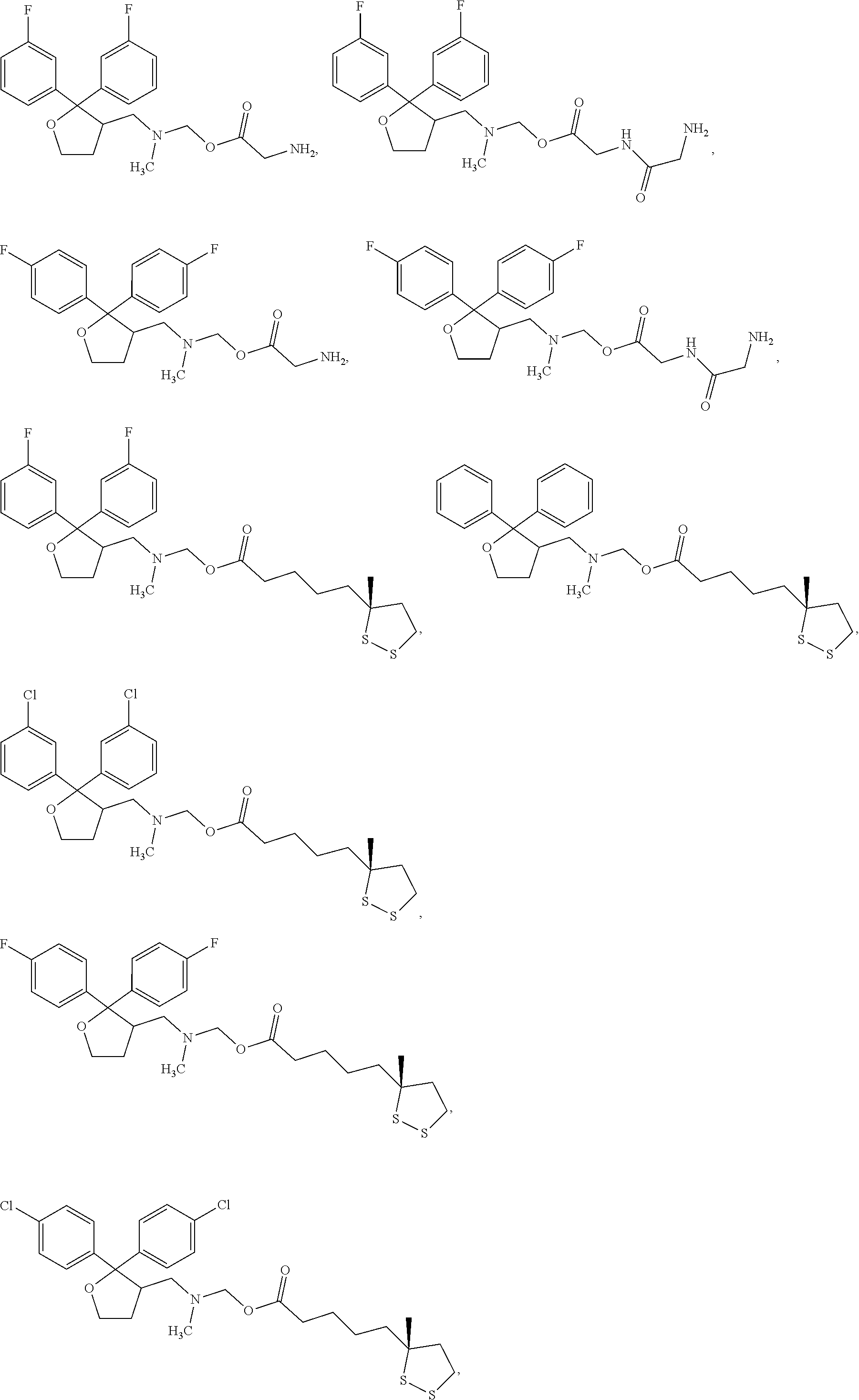

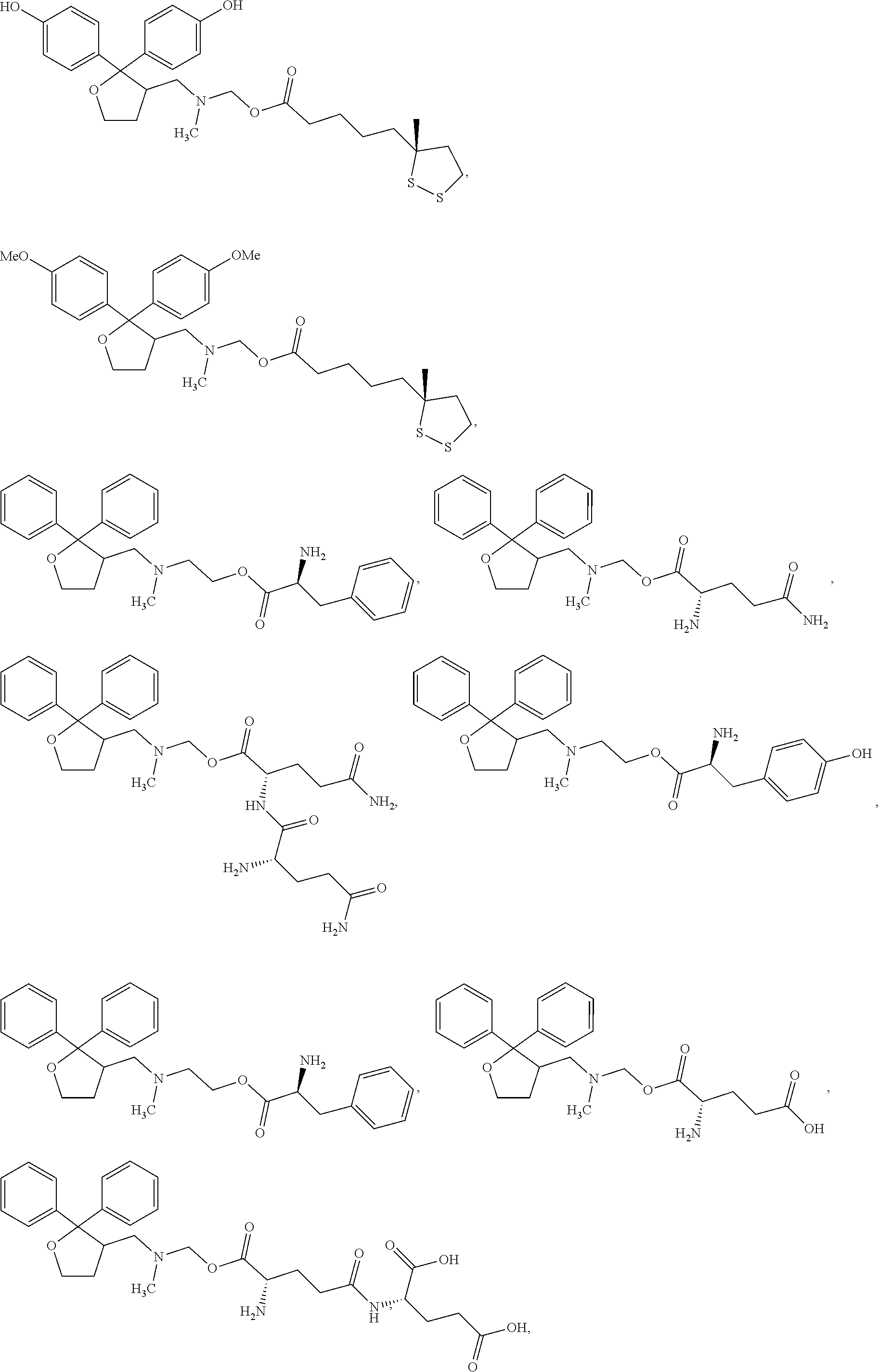

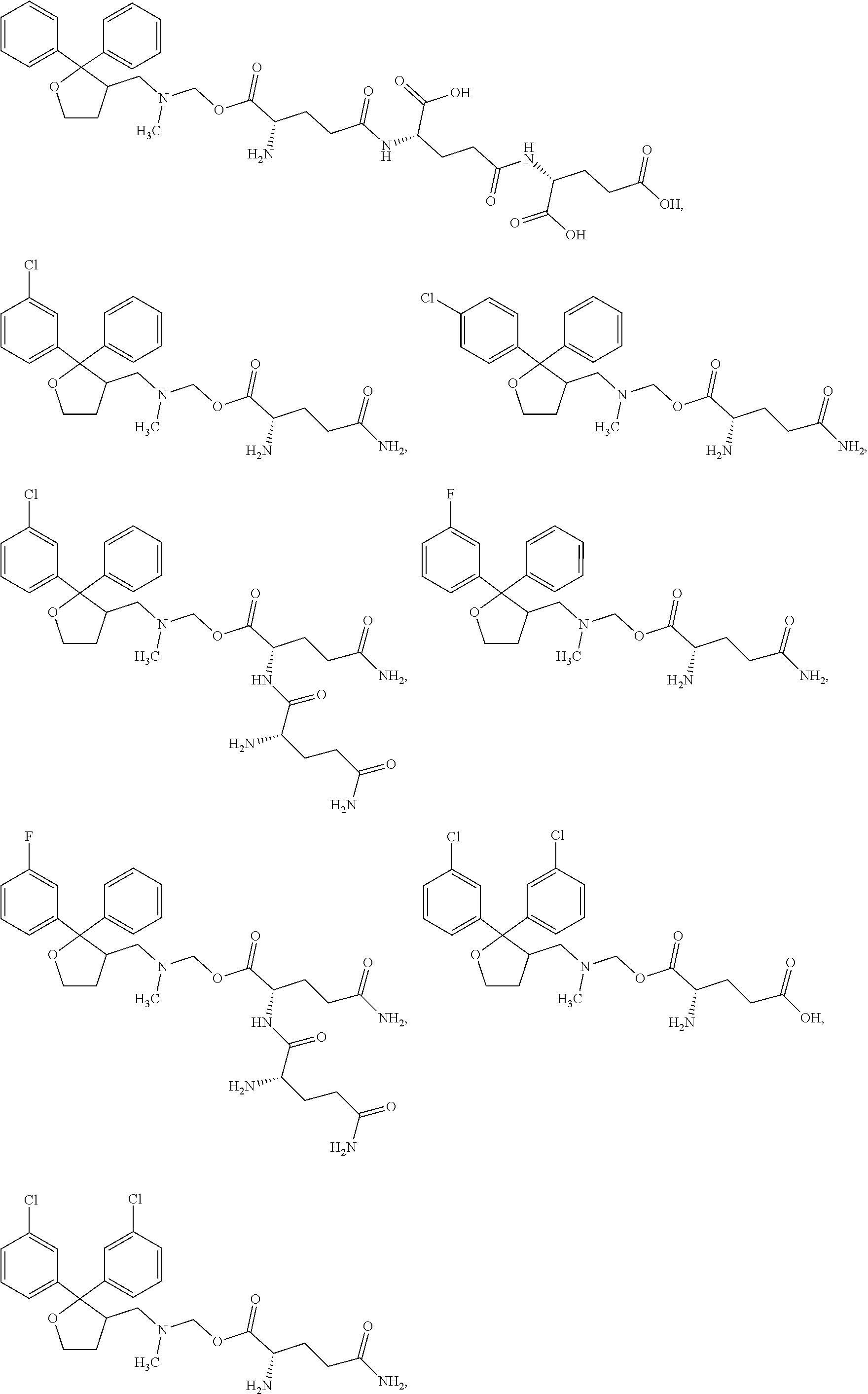

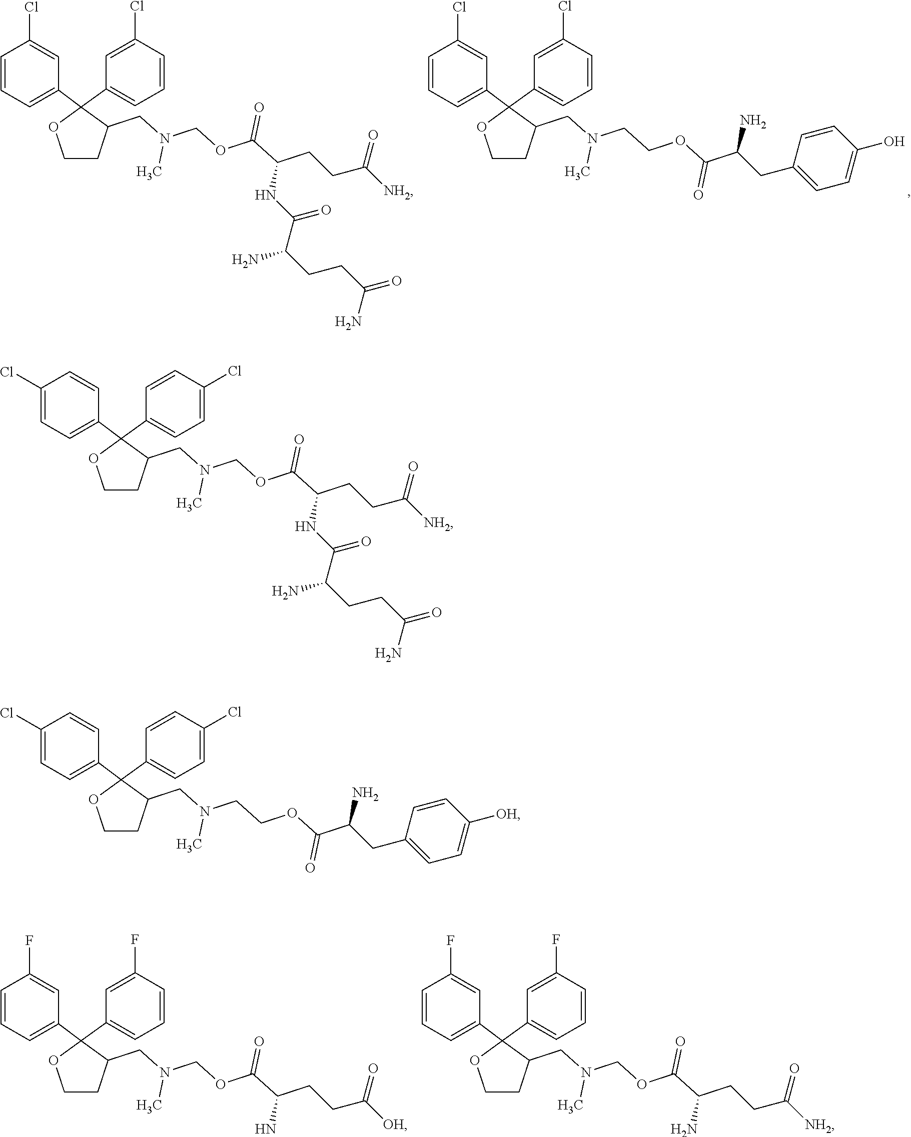 [SMILES: CN(COC(=O)[C@H](CCC(N)=O)NC(=O)[C@@H](N)CCC(N)=O)CC1CCOC1(c1cccc(Cl)c1)c1cccc(Cl)c1 ; CN(CCOC(=O)[C@@H](N)Cc1ccc(O)cc1)CC1CCOC1(c1cccc(Cl)c1)c1cccc(Cl)c1 ; CN(COC(=O)[C@H](CCC(N)=O)NC(=O)[C@@H](N)CCC(N)=O)CC1CCOC1(c1ccc(Cl)cc1)c1ccc(Cl)cc1 ; CN(CCOC(=O)[C@@H](N)Cc1ccc(O)cc1)CC1CCOC1(c1ccc(Cl)cc1)c1ccc(Cl)cc1 ; CN(COC(=O)[C@@H](N)CCC(=O)O)CC1CCOC1(c1cccc(F)c1)c1cccc(F)c1 ; CN(COC(=O)[C@@H](N)CCC(N)=O)CC1CCOC1(c1cccc(F)c1)c1cccc(F)c1]

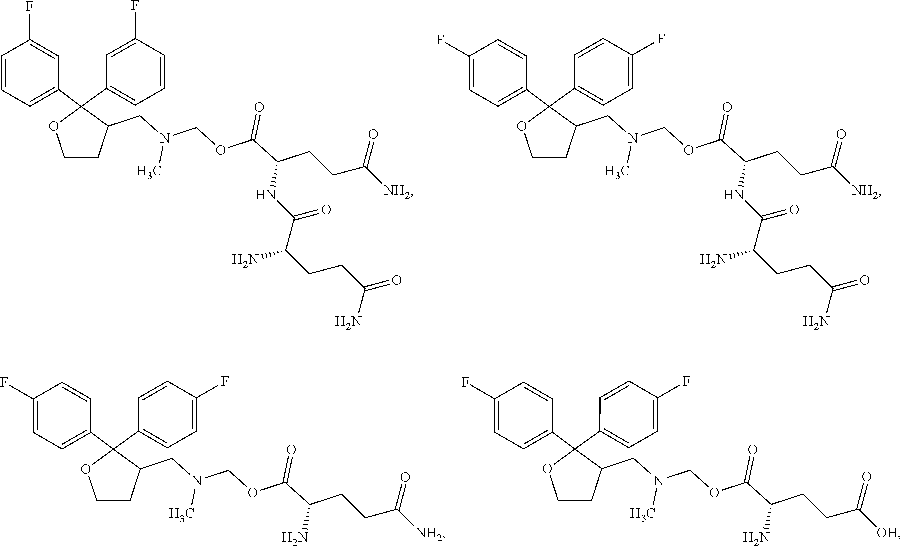 [SMILES: CN(COC(=O)[C@H](CCC(N)=O)NC(=O)[C@@H](N)CCC(N)=O)CC1CCOC1(c1cccc(F)c1)c1cccc(F)c1 ; CN(COC(=O)[C@H](CCC(N)=O)NC(=O)[C@@H](N)CCC(N)=O)CC1CCOC1(c1ccc(F)cc1)c1ccc(F)cc1 ; CN(COC(=O)[C@@H](N)CCC(N)=O)CC1CCOC1(c1ccc(F)cc1)c1ccc(F)cc1 ; CN(COC(=O)[C@@H](N)CCC(=O)O)CC1CCOC1(c1ccc(F)cc1)c1ccc(F)cc1]

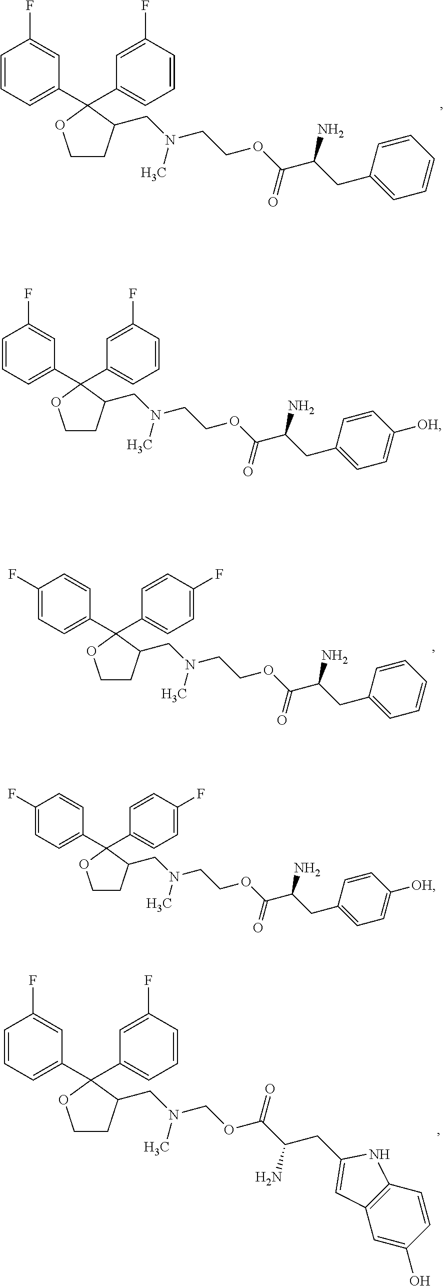 [SMILES: CN(CCOC(=O)[C@@H](N)Cc1ccccc1)CC1CCOC1(c1cccc(F)c1)c1cccc(F)c1 ; CN(CCOC(=O)[C@@H](N)Cc1ccc(O)cc1)CC1CCOC1(c1cccc(F)c1)c1cccc(F)c1 ; CN(CCOC(=O)[C@@H](N)Cc1ccccc1)CC1CCOC1(c1ccc(F)cc1)c1ccc(F)cc1 ; CN(CCOC(=O)[C@@H](N)Cc1ccc(O)cc1)CC1CCOC1(c1ccc(F)cc1)c1ccc(F)cc1 ; CN(COC(=O)[C@@H](N)Cc1cc2cc(O)ccc2[nH]1)CC1CCOC1(c1cccc(F)c1)c1cccc(F)c1]

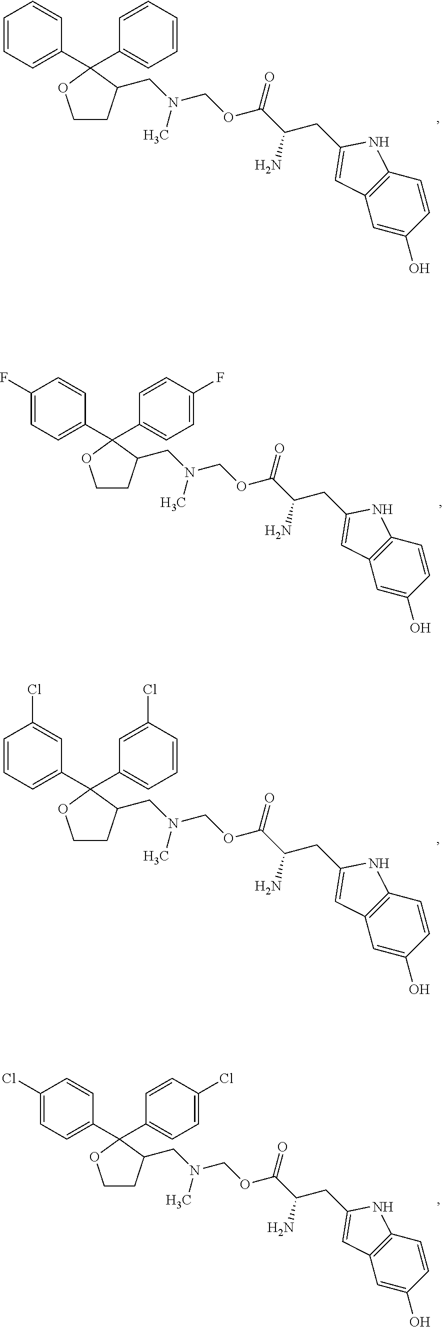 [SMILES: CN(COC(=O)[C@@H](N)Cc1cc2cc(O)ccc2[nH]1)CC1CCOC1(c1ccccc1)c1ccccc1 ; CN(COC(=O)[C@@H](N)Cc1cc2cc(O)ccc2[nH]1)CC1CCOC1(c1ccc(F)cc1)c1ccc(F)cc1 ; CN(COC(=O)[C@@H](N)Cc1cc2cc(O)ccc2[nH]1)CC1CCOC1(c1cccc(Cl)c1)c1cccc(Cl)c1 ; CN(COC(=O)[C@@H](N)Cc1cc2cc(O)ccc2[nH]1)CC1CCOC1(c1ccc(Cl)cc1)c1ccc(Cl)cc1]

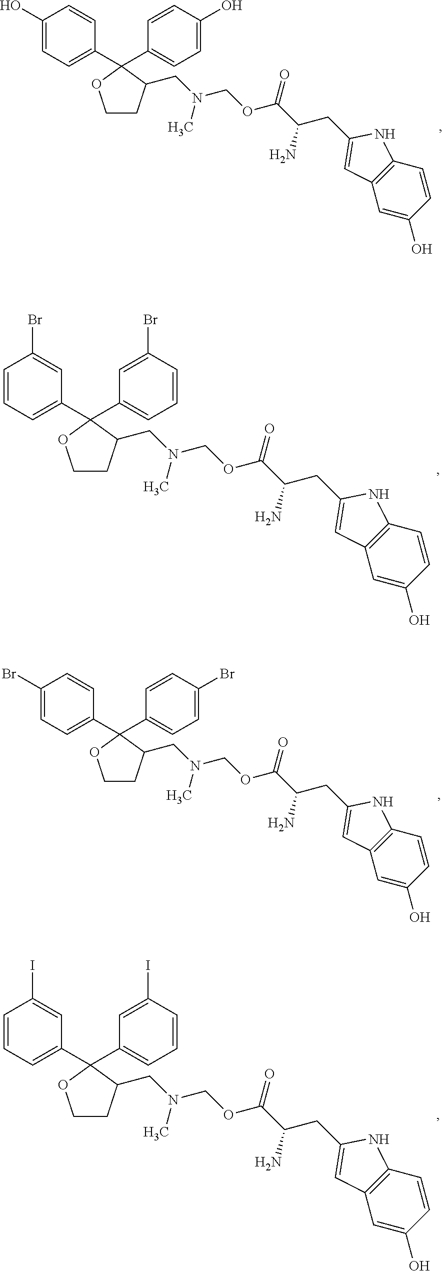 [SMILES: CN(COC(=O)[C@@H](N)Cc1cc2cc(O)ccc2[nH]1)CC1CCOC1(c1ccc(O)cc1)c1ccc(O)cc1 ; CN(COC(=O)[C@@H](N)Cc1cc2cc(O)ccc2[nH]1)CC1CCOC1(c1cccc(Br)c1)c1cccc(Br)c1 ; CN(COC(=O)[C@@H](N)Cc1cc2cc(O)ccc2[nH]1)CC1CCOC1(c1ccc(Br)cc1)c1ccc(Br)cc1 ; CN(COC(=O)[C@@H](N)Cc1cc2cc(O)ccc2[nH]1)CC1CCOC1(c1cccc(I)c1)c1cccc(I)c1]

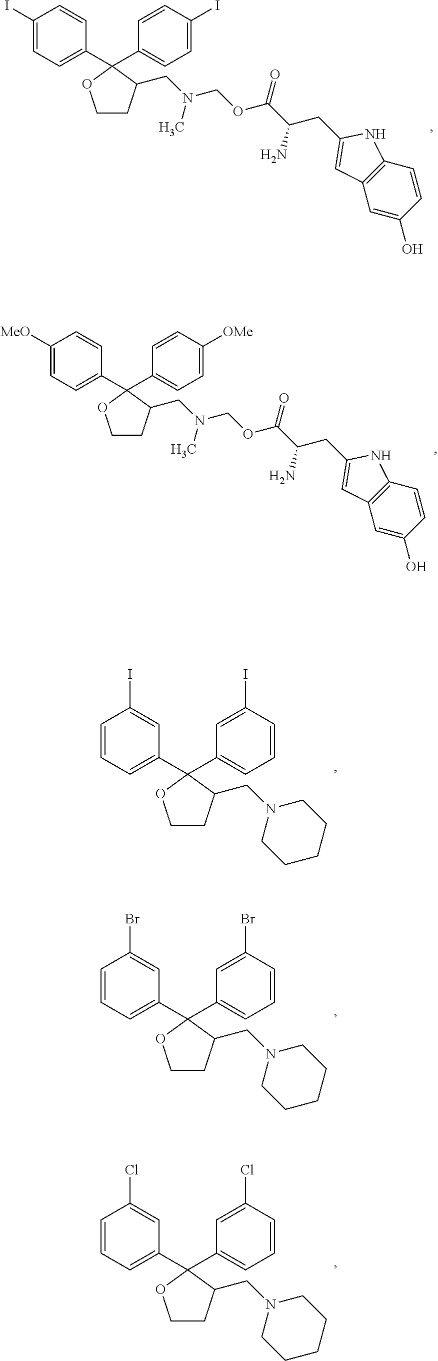 [SMILES: CN(COC(=O)[C@@H](N)Cc1cc2cc(O)ccc2[nH]1)CC1CCOC1(c1ccc(I)cc1)c1ccc(I)cc1 ; COc1ccc(C2(c3ccc(OC)cc3)OCCC2CN(C)COC(=O)[C@@H](N)Cc2cc3cc(O)ccc3[nH]2)cc1 ; Ic1cccc(C2(c3cccc(I)c3)OCCC2CN2CCCCC2)c1 ; Brc1cccc(C2(c3cccc(Br)c3)OCCC2CN2CCCCC2)c1 ; Clc1cccc(C2(c3cccc(Cl)c3)OCCC2CN2CCCCC2)c1]

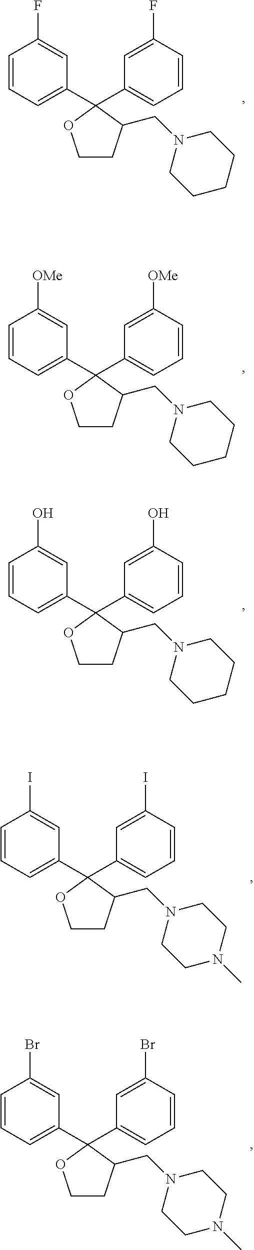 [SMILES: Fc1cccc(C2(c3cccc(F)c3)OCCC2CN2CCCCC2)c1 ; COc1cccc(C2(c3cccc(OC)c3)OCCC2CN2CCCCC2)c1 ; Oc1cccc(C2(c3cccc(O)c3)OCCC2CN2CCCCC2)c1 ; CN1CCN(CC2CCOC2(c2cccc(I)c2)c2cccc(I)c2)CC1 ; CN1CCN(CC2CCOC2(c2cccc(Br)c2)c2cccc(Br)c2)CC1]

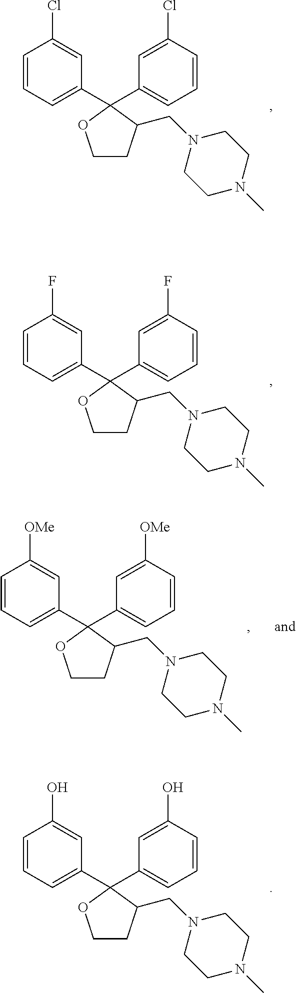 [SMILES: CN1CCN(CC2CCOC2(c2cccc(Cl)c2)c2cccc(Cl)c2)CC1 ; CN1CCN(CC2CCOC2(c2cccc(F)c2)c2cccc(F)c2)CC1 ; COc1cccc(C2(c3cccc(OC)c3)OCCC2CN2CCN(C)CC2)c1 ; CN1CCN(CC2CCOC2(c2cccc(O)c2)c2cccc(O)c2)CC1]

13. A pharmaceutical composition for treating Sigma-1 receptor related disorders or diseases, comprising the compound of claim 1.
